(19) United States
(12) Patent Application Publication
(10) Pub. No.: US 2023/0027495 A1
(43) Pub. Date: Jan. 26, 2023

(54) METHODS OF TREATING CANCER WITH A COMBINATION OF AN ANTI-PD-1 ANTIBODY AND AN ANTI-TISSUE FACTOR ANTIBODY-DRUG CONJUGATE

(71) Applicants: Genmab A/S, Copenhagen V (DK); MSD International GmbH, Lucerne (CH)

(72) Inventors: Reshma Abdulla RANGWALA, Philadelphia, PA (US); Esther C.W. BREIJ, Utrecht (NL); Sandra VERPLOEGEN, Utrecht (NL); Oyewale O. ABIDOYE, Bothell, WA (US); Leonardo Viana NICACIO, Bothell, WA (US); Anthony CAO, Bothell, WA (US); Shyra GARDAI, Bothell, WA (US)

(73) Assignees: Genmab A/S, Copenhagen V (DK); MSD International GmbH, Lucerne (CH)

(21) Appl. No.: 17/775,279

(22) PCT Filed: Nov. 6, 2020
(86) PCT No.: PCT/IB2020/060485

§ 371 Date: May 7, 2022

Related U.S. Application Data
(60) Provisional application No. 62/932,377, filed on Nov. 7, 2019.

(51) Int. Cl. A61K 47/68; C07K 16/28; C07K 16/36; A61P 35/00

(52) U.S. Cl. CPC A61K 47/6849; A61K 47/6851; A61K 47/6889; C07K 16/2818; C07K 16/36; A61P 35/00; A61K 2039/507

(57) ABSTRACT

The invention provides an anti-PD-1 antibody comprising the complementary determining regions (CDRs) of pembrolizumab in combination with an antibody-drug conjugate that binds to tissue factor (TF) comprising monomethyl auristatin E and the CDRs of tisotumab (e.g., tisotumab vedotin) and their use in methods of treating cancer, such as breast cancer and cervical cancer. The invention also provides compositions and kits comprising the anti-PD-1 antibody comprising the CDRs of pembrolizumab and the antibody-drug conjugate that binds to TF comprising monomethyl auristatin E and the CDRs of tisotumab (e.g., tisotumab vedotin) for use in treating cancer, such as breast cancer and cervical cancer.

### REFERENCE TO RELATED APPLICATIONS

This application claims the benefit of U.S. Provisional Application No. 62/932,377 filed on Nov. 7, 2019, the contents of which are hereby incorporated in their entirety.

### TECHNICAL FIELD

The present invention relates to methods of treating cancer, such as breast cancer and cervical cancer, with a combination of an anti-PD-1 antibody comprising the complementary determining regions (CDRs) of pembrolizumab and an anti-Tissue Factor (anti-TF) antibody-drug conjugate, wherein the antibody-drug conjugate comprises an anti-TF antibody or an antigen-binding fragment thereof comprising the CDRs of tisotumab conjugated to monomethyl auristatin E (MMAE).

### BACKGROUND

Tissue factor (TF), also called thromboplastin, factor III or CD142 is a protein present in subendothelial tissue, platelets, and leukocytes necessary for the initiation of thrombin formation from the zymogen prothrombin. Thrombin formation ultimately leads to the coagulation of blood. TF enables cells to initiate the blood coagulation cascades, and it functions as the high-affinity receptor for the coagulation factor VII (FVII), a serine protease. The resulting complex provides a catalytic event that is responsible for initiation of the coagulation protease cascades by specific limited proteolysis. Unlike the other cofactors of these protease cascades, which circulate as nonfunctional precursors, TF is a potent initiator that is fully functional when expressed on cell surfaces.

TF is the cell surface receptor for the serine protease factor VIIa (FVIIa). Binding of FVIIa to TF starts signaling processes inside the cell, said signaling function playing a role in angiogenesis. Whereas angiogenesis is a normal process in growth and development, as well as in wound healing, it is also a fundamental step in the transition of tumors from a dormant state to a malignant state. When cancer cells gain the ability to produce proteins that participate in angiogenesis (i.e., angiogenic growth factors), these proteins are released by the tumor into nearby tissues, thereby stimulating new blood vessels to sprout from existing healthy blood vessels toward and into the tumor. Once new blood vessels enter the tumor, the tumor can rapidly expand its size and invade local tissue and organs. Through the new blood vessels, cancer cells may further escape into the circulation and lodge in other organs to form new tumors, also known as metastasis.

TF expression is observed in many types of cancer, including cervical cancer, and is associated with more aggressive disease. Furthermore, human TF also exists in a soluble alternatively-spliced form, asHTF. It has recently been found that asHTF promotes tumor growth (Hobbs et al., 2007, Thrombosis Res. 120(2):S13-S21).

Human cancers harbor numerous genetic and epigenetic alterations, generating neoantigens potentially recognizable by the immune system (Sjoblom et al., 2006, Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities. Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of an antibody, ipilimumab (YERVOY®), that binds to and inhibits CTLA-4 for the treatment of patients with advanced melanoma (Hodi et al., 2010, N Engl J Med 363:711-23) and the development of an antibody, pembrolizumab (formerly lambrolizumab; USAN Council Statement, 2013), that binds specifically to the Programmed Death-1 (PD-1) receptor and block the inhibitory PD-1/PD-1 ligand pathway (Hamid and Carvajal, Expert Opin Biol Ther 13(6):847-61 (2013); and McDermott and Atkins, Cancer Med 2(5):662-73 (2013)).

Breast cancer is by far the most common cancer among women. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease (National Cancer Institute, Surveillance Epidemiology and End Results (NCI SEER) program, Cancer Statistics Review (CSR), www-seer.ims.nci.nih.gov/Publications/CSR1973 (1998)). Based on cancer rates from 1995 through 1997, a report from the National Cancer Institute (NCI) estimates that about 1 in 8 women in the United States (approximately 12.8 percent) will develop breast cancer during her lifetime (NCI's Surveillance, Epidemiology, and End Results Program (SEER) publication SEER Cancer Statistic's Review 1973-1997). Breast cancer is the second most common form of cancer, after skin cancer, among women in the United States. An estimated 250,100 new cases of breast cancer are expected to be diagnosed in the United States in 2001. Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & FIGS. 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMD-92-12; 1991). Only 5 to 10% of breast cancers are linked to a family history of breast cancer (Henderson I C, Breast Cancer. In: Murphy G P, Lawrence W L, Lenhard R E (eds). Clinical Oncology. Atlanta, Ga.: American Cancer Society; 1995:198-219).

Cervical cancer poses a significant medical problem worldwide with an estimated incidence of more than 500,000 new cases and 250,000 deaths annually. See Tewari et al., 2014, N Engl J Med., 370:734-743. In the Europe Union, approximately 34,000 new cases of cervical cancer and 13,000 deaths occur annually. See Hillemanns et al., 2016, Oncol. Res. Treat. 39:501-506. The main types of cervical cancer are squamous cell carcinoma and adenocarcinoma. Long-lasting infections with human papillomavirus (HPV) type 16 and 18 cause most cases of cervical cancer. The standard for first-line therapy of cervical cancer was a platinum-based therapy plus a taxane-based therapy. Bevacizumab, an anti-VEGF antibody, was approved by the U.S. Food and Drug Administration for use in combination with chemotherapy for the treatment of cervical cancer, which had improved overall survival in clinical trials. First-line (1L) treatment for advanced cervical cancer is comprised of bevacizumab combined with paclitaxel plus a platinum (e.g., cisplatin or carboplatin) or paclitaxel plus topotecan. Despite a 48% objective response rate (ORR) and a median overall survival (OS) of approximately 18 months, unfortunately almost all patients relapse after this 1L treatment. See Tewari et al., 2014, N Engl J Med., 370:734-743. In 2018, pembrolizumab (anti-programmed death 1 antibody) received accelerated approval in the United States for the 2L+ treatment of patients with programmed death-ligand 1 (PD-L1)-positive (combined positive score ≥1%) recurrent or metastatic cervical cancer (r/mCC). The objective response rate (ORR) of pembrolizumab was 14% in this setting where 42% of patients had been previously treated with bevacizumab. See Corp. MSD. KEYTRUDA® (pembrolizumab) for injection, for intravenous use. Whitehouse Station, N.J.: Merck & Co., Inc.; June 2018. Most patients enrolled in the study had squamous histology (92%) (Id.), and therefore, little is known on the efficacy of pembrolizumab in patients with non-squamous histology. The majority of second-line (and beyond) patients with recurrent or metastatic cervical cancer do not benefit from treatment with pembrolizumab. These data highlight the immediate need for more effective therapies that provide clinical benefits across a broader r/mCC patient population previously treated with doublet chemotherapy with or without bevacizumab and not restricted by biomarker expression. For second-line (2L) treatment, patients are often treated with single agent modalities including, but not limited to: pemetrexed, topotecan, docetaxel, nab-paclitaxel, vinorelbine and in some cases bevacizumab. A meta-analysis of single agent treatment demonstrates a modest response rate of only 10.9% (i.e., 60 responders out of 552 patients) and median overall survivals (OS) of approximately 7 months. See e.g., Burotto et al., 2015, Oncologist 20:725-726; Candelaria et al., 2009, Int. J. Gynecol. Cancer. 19:1632-1637; Coronel et al., 2009, Med. Oncol. 26:210-214; Fiorica et al., 2009, Gynecol. Oncol. 115:285-289; Garcia et. al., 2007, Am. J. Clin. Oncol. 30-428-431; Goncalves et al., 2008, Gynecol. Oncol. 108:42-46; Homesley et al., 2008, Int. J. Clin. Oncol. 13:62-65; McLachlan et al., 2017, Clin. Oncol. (R. Coll. Radiol.) 29:153-160; Miller et al., 2008, Gynecol. Oncol. 110:65-70; Monk et al., 2009, J. Clin. Oncol. 27:1069-1074; Muggia et al., 2004, Gynecol. Oncol. 92:639-643; Rose et al., 2006, Gynecol. Oncol. 102:210-213; Santin et al., 2011, Gynecol. Oncol. 122:495-500; Schilder et al., 2005, Gynecol. Oncol. 96:103-107; and Torfs et al., 2012, Eur. J. Cancer. 48:1332-1340. The five year relative survival for stage IV cervical cancer is only 15%, demonstrating a high need for improved therapy against cervical cancer.

Targeted therapy of multiple non-redundant molecular pathways regulating immune responses can enhance antitumor immunotherapy. However, not all combinations have acceptable safety and/or efficacy. There remains a need for combination therapies with an acceptable safety profile and high efficacy for the treatment of cancer, in particular for the treatment of breast cancer and cervical cancer.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating cancer in a subject comprising administering to the subject an antibody or an antigen-binding fragment thereof, wherein the antibody binds to Programmed Death-1 (PD-1) and inhibits PD-1 activity, and an antibody-drug conjugate that binds to tissue factor (TF), wherein the antibody-drug conjugate comprises an anti-TF antibody or an antigen-binding fragment thereof conjugated to monomethyl auristatin E, wherein the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO: 17;
(ii) a CDR-H2 comprising the amino acid sequence of SEQ ID NO: 18; and
(iii) a CDR-H3 comprising the amino acid sequence of SEQ ID NO: 19; and
   wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22, wherein the CDRs of the anti-PD-1 antibody or antigen-binding fragment thereof are generally defined by the Kabat numbering scheme,
   and wherein the anti-TF antibody or antigen-binding fragment thereof comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO:2; and
(iii) a CDR-H3 comprising the amino acid sequence of SEQ ID NO: 3; and
   wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6, wherein the CDRs of the anti-TF antibody or antigen-binding fragment thereof are defined by the IMGT numbering scheme, wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period. In some embodiments, the antibody-drug conjugate is administered at a dose of about 0.65 mg/kg. In some embodiments, the antibody-drug conjugate is administered at a dose of 0.65 mg/kg. In some embodiments, the antibody-drug conjugate is administered at a dose of about 0.7 mg/kg. In some embodiments, the antibody-drug conjugate is administered at a dose of 0.7 mg/kg. In some embodiments, the antibody-drug conjugate is administered at a dose of about 0.8 mg/kg. In some embodiments, the antibody-drug conjugate is administered at a dose of 0.8 mg/kg. In some embodiments, the antibody-drug conjugate is administered at a dose of about 0.9 mg/kg. In some embodiments, the antibody-drug conjugate is administered at a dose of 0.9 mg/kg. In some embodiments, the antibody-drug conjugate is administered at a dose of about 1.0 mg/kg. In some embodiments, the antibody-drug conjugate is administered at a dose of 1.0 mg/kg. In some embodiments, the antibody-drug conjugate is administered at a dose of about 1.1 mg/kg. In some embodiments, the antibody-drug conjugate is administered at a dose of 1.1 mg/kg. In some embodiments, the antibody-drug conjugate is administered at a dose of about 1.2 mg/kg. In some embodiments, the antibody-drug conjugate is administered at a dose of 1.2 mg/kg. In some embodiments, the antibody-drug conjugate is administered at a dose of about 1.3 mg/kg. In some embodiments, the antibody-drug conjugate is administered at a dose of 1.3 mg/kg. In some embodiments, the antibody-drug conjugate is administered at a dose of about 1.4 mg/kg. In some embodiments, the antibody-drug conjugate is administered at a dose of 1.4 mg/kg. In some embodiments, the antibody-drug conjugate is administered at a dose of about 1.5 mg/kg. In some embodiments, the antibody-drug conjugate is administered at a dose of 1.5 mg/kg. In some of any of the embodiments herein, the antibody-drug conjugate is administered once every 1 week for 3 consecutive weeks followed by a 1 week rest period without any administration of the antibody-drug conjugate so that each cycle time is 28 days including the resting period. In some of any of the embodiments herein, the antibody-drug conjugate is administered on about days 1, 8, and 15 of about a 4-week cycle. In some of any of the embodiments herein, the antibody-drug conjugate is administered on days 1, 8, and 15 of a 4-week cycle. In some of any of the embodiments herein, the anti-PD-1 antibody or antigen-binding fragment thereof is administered at a flat dose ranging from about 50 mg to about 500 mg. In some of any of the embodiments herein, the anti-PD-1 antibody or antigen-binding fragment thereof is administered at a flat dose of about 200 mg. In some of any of the embodiments herein, the anti-PD-1 antibody or antigen-binding fragment thereof is administered at a flat dose of 200 mg. In some of any of the embodiments herein, the anti-PD-1 antibody or antigen-binding fragment thereof is administered at a flat dose of about 400 mg. In some of any of the embodiments herein, the anti-PD-1 antibody or antigen-binding fragment thereof is administered at a flat dose of 400 mg. In some of any of the embodiments herein, the anti-PD-1 antibody or antigen-binding fragment thereof is administered once about every 1 week, once about every 2 weeks, once about every 3 weeks, once about every 4 weeks, once about every 5 weeks, or once about every 6 weeks. In some of any of the embodiments herein, the anti-PD-1 antibody or antigen-binding fragment thereof is administered once about every 3 weeks. In some of any of the embodiments herein, the anti-PD-1 antibody or antigen-binding fragment thereof is administered once every 3 weeks. In some of any of the embodiments herein, the anti-PD-1 antibody or antigen-binding fragment thereof is administered once about every 6 weeks. In some of any of the embodiments herein, the anti-PD-1 antibody or antigen-binding fragment thereof is administered once every 6 weeks. In some of any of the embodiments herein, the anti-PD-1 antibody or antigen-binding fragment thereof is administered on about day 1 of about a 21-day cycle. In some of any of the embodiments herein, the anti-PD-1 antibody or antigen-binding fragment thereof is administered on day 1 of a 21-day cycle. In some of any of the embodiments herein, the anti-PD-1 antibody or antigen-binding fragment thereof is administered on about day 1 of about a 6-week cycle. In some of any of the embodiments herein, the anti-PD-1 antibody or antigen-binding fragment thereof is administered on day 1 of a 6-week cycle. In some of any of the embodiments herein, the cancer is breast cancer. In some of any of the embodiments herein, the cancer is cervical cancer. In some of any of the embodiments herein, the subject is not a candidate for curative therapy. In some of any of the embodiments herein, the curative therapy comprises radiotherapy and/or exenterative surgery. In some of any of the embodiments herein, the subject has not received prior systemic therapy for the cervical cancer. In some of any of the embodiments herein, the cervical cancer is a non-squamous cell carcinoma, an adenocarcinoma, an adenosquamous carcinoma or a squamous cell carcinoma. In some of any of the embodiments herein, the cervical cancer is a non-squamous cell carcinoma. In some of any of the embodiments herein, the cervical cancer is an adenocarcinoma. In some of any of the embodiments herein, the cervical cancer is an adenosquamous carcinoma. In some of any of the embodiments herein, the cervical cancer is a squamous cell carcinoma. In some of any of the embodiments herein, the cervical cancer is an advanced stage cervical cancer. In some of any of the embodiments herein, the advanced stage cervical cancer is a stage 3 or stage 4 cervical cancer. In some of any of the embodiments herein, the advanced stage cervical cancer is metastatic cervical cancer. In some of any of the embodiments herein, the cervical cancer is recurrent cervical cancer. In some of any of the embodiments herein, the anti-TF antibody or antigen-binding fragment thereof of the antibody-drug conjugate is a monoclonal antibody or a monoclonal antigen-binding fragment thereof. In some of any of the embodiments herein, the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8. In some of any of the embodiments herein, the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8. In some of any of the embodiments herein, the anti-TF antibody of the antibody-drug conjugate is tisotumab. In some of any of the embodiments herein, the antibody-drug conjugate further comprises a linker between the anti-TF antibody or antigen-binding fragment thereof and the monomethyl auristatin E. In some of any of the embodiments herein, the linker is a cleavable peptide linker. In some of any of the embodiments herein, the cleavable peptide linker has a formula: -MC-vc-PAB-, wherein:

a) MC is:

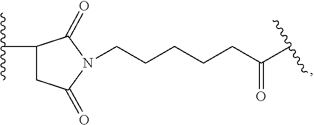 [SMILES: *C(=O)CCCCCN1C(=O)CC(*)C1=O]

b) vc is the dipeptide valine-citrulline, and

c) PAB is:

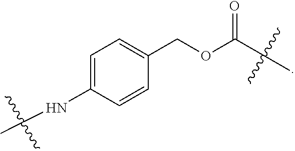

In some of any of the embodiments herein, the linker is attached to sulphydryl residues of the anti-TF antibody or antigen-binding fragment thereof obtained by partial reduction or full reduction of the anti-TF antibody or antigen-binding fragment thereof. In some of any of the embodiments herein, the linker is attached to MMAE, wherein the antibody-drug conjugate has the following structure:

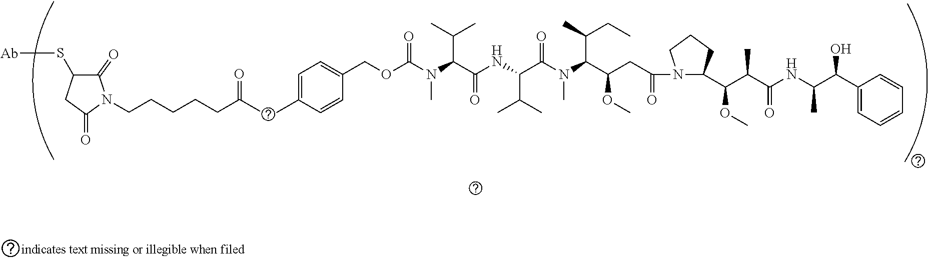

wherein p denotes a number from 1 to 8, S represents a sulphydryl residue of the anti-TF antibody, and Ab designates the anti-TF antibody or antigen-binding fragment thereof. In some of any of the embodiments herein, the average value of p in a population of the antibody-drug conjugates is about 4. In some of any of the embodiments herein, the antibody-drug conjugate is tisotumab vedotin. In some of any of the embodiments herein, the route of administration for the antibody-drug conjugate is intravenous. In some of any of the embodiments herein, the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32. In some of any of the embodiments herein, the anti-PD-1 antibody comprises a heavy chain variable region comprising the amino acid sequence of SEQ ID NO:31 and a light chain variable region comprising the amino acid sequence of SEQ ID NO:32. In some of any of the embodiments herein, the anti-PD-1 antibody comprises a heavy chain comprising the amino acid sequence of SEQ ID NO:33 and a light chain comprising the amino acid sequence of SEQ ID NO:34. In some of any of the embodiments herein the anti-PD-1 antibody is pembrolizumab. In some of any of the embodiments herein, the route of administration for the anti-PD-1 antibody or antigen-binding fragment thereof is intravenous. In some of any of the embodiments herein, the route of administration for the anti-PD-1 antibody or antigen-binding fragment thereof is subcutaneous. In some of any of the embodiments herein, the anti-PD-1 antibody or antigen-binding fragment thereof and the antibody-drug conjugate are administered sequentially. In some of any of the embodiments herein, the anti-PD-1 antibody or antigen-binding fragment thereof and the antibody-drug conjugate are administered simultaneously. In some of any of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF. In some of any of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1. In some of any of the embodiments herein, the subject's tumor expresses PD-L1 with a tumor proportion score (TPS)≥1%. In some of any of the embodiments herein, the subject's tumor has high PD-L1 expression (TPS≥50%). In some of any of the embodiments herein, the subject's tumor expresses PD-L1 with a combined positive score (CPS)≥1%. In some of any of the embodiments herein, the subject's tumor expresses PD-L1 with a combined positive score (CPS)≥10%. In some of any of the embodiments herein, a tumor derived from the cancer comprises one or more cells that express PD-L1, PD-L2, or both PD-L1 and PD-L2. In some of any of the embodiments herein, at least about 0.10%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some of any of the embodiments herein, one or more therapeutic effects in the subject is improved after administration of the antibody-drug conjugate and the anti-PD-1 antibody or antigen-binding fragment thereof relative to a baseline. In some of any of the embodiments herein, the one or more therapeutic effects is selected from the group consisting of: size of a tumor derived from the cancer, objective response rate, duration of response, time to response, progression free survival, and overall survival. In some of any of the embodiments herein, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof. In some of any of the embodiments herein, the objective response rate is at least about 20%, at least about 25%, at least about 30%, at least about 35%, at least about 40%, at least about 45%, at least about 50%, at least about 60%, at least about 70%, or at least about 80%. In some of any of the embodiments herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof. In some of any of the embodiments herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof. In some of any of the embodiments herein, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof. In some of any of the embodiments herein, the subject has one or more adverse events and is further administered an additional therapeutic agent to eliminate or reduce the severity of the one or more adverse events. In some of any of the embodiments herein, the subject is at risk of developing one or more adverse events and is further administered an additional therapeutic agent to prevent or reduce the severity of the one or more adverse events. In some of any of the embodiments herein, the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration. In some of any of the embodiments herein, the one or more adverse events is a grade 3 or greater adverse event. In some of any of the embodiments herein, the one or more adverse events is a serious adverse event. In some of any of the embodiments herein, the one or more adverse events is conjunctivitis, conjunctival ulceration, and/or keratitis and the additional agent is a preservative-free lubricating eye drop, an ocular vasoconstrictor, antibiotic, and/or a steroid eye drop. In some of any of the embodiments herein, the subject is a human. In some of any of the embodiments herein, the antibody-drug conjugate is in a pharmaceutical composition comprising the antibody-drug conjugate and a pharmaceutical acceptable carrier. In some of any of the embodiments herein, the anti-PD-1 antibody or antigen-binding fragment thereof is in a pharmaceutical composition comprising the anti-PD-1 antibody or antigen-binding fragment thereof and a pharmaceutical acceptable carrier.

Also provided herein are kits comprising:

(a) a dosage ranging from about 50 mg to about 500 mg of an antibody or an antigen-binding fragment thereof, wherein the antibody binds to Programmed Death-1 (PD-1) and inhibits PD-1 activity, wherein the antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are generally defined by the Kabat numbering scheme;

(b) a dosage ranging from about 0.5 mg to about 200 mg of an antibody-drug conjugate that binds to tissue factor (TF), wherein the antibody-drug conjugate comprises an anti-TF antibody or an antigen-binding fragment thereof conjugated to monomethyl auristatin E, wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO: 3; and wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme; and

(c) instructions for use of the anti-PD-1 antibody or antigen-binding fragment thereof and the antibody drug conjugate according to some of any of the embodiments herein. In some of any of the embodiments herein, the anti-PD-1 antibody or antigen-binding fragment thereof is pembrolizumab. In some of any of the embodiments herein, the dose of the pembrolizumab is 200 mg. In some of any of the embodiments herein, the dose of the pembrolizumab is 400 mg. In some of any of the embodiments herein, the antibody-drug conjugate is tisotumab vedotin.

### BRIEF DESCRIPTION OF THE DRAWINGS

FIG. 1 is an image of a Western blot showing phosphorylation of IRE1 and JNK in cell lysates of HeLa cells treated with MMAE (right lane) as compared to HeLa cells not treated with MMAE (left lane). Treatment with MMAE led to phosphorylation of both IRE1 and JNK. pIRE1 indicates phosphorylated IRE1 protein; IRE1 indicated total IRE1 protein; and pJNK indicates phosphorylated JNK protein.

FIGS. 2A and 2B are immunofluorescent images of HeLa cells treated with 100 nM MMAE and imaged at the indicated time points in the presence of MMAE. A) Top panel shows staining for the ER with the ER-binding dye ER-1D Green and B) the lower panel shows RFP-labeled tubulin expressed by the cells

FIGS. 3A and 3B is a series of graphs showing A) ATP secretion and B) HMGB1 secretion from HeLa cells treated with 100 nM MMAE as compared to HeLa cells not treated with MMAE. Measurements were treated HeLa cells are shown as the fold change over the signal produced by untreated HeLa cells. **p<0.01 and ****p<0.0001.

FIGS. 4A and 4B is a series of graphs showing the anti-tumor activity of the combination of tisotumab vedotin and pembrolizumab in an MDA-MB-231 xenograft model in humanized mice. A) Average tumor size in the MDA-MB-231 xenograft model in NSG mice after treatment with IgG1 control (empty circle), IgG1-MMAE control (empty diamond), pembrolizumab (filled circle), tisotumab vedotin at a concentration of 0.5 mg/kg (half-filled triangle) or 1 mg/kg (half-filled square), or tisotumab vedotin at a concentration of 0.5 mg/kg (filled triangle) or 1 mg/kg (filled square) combined with pembrolizumab. Inverted empty triangle indicates day of administration of pembrolizumab dose. Inverted half-filled triangle indicates day of administration of IgG1 control, IgG1-MMAE control or tisotumab vedotin dose. Tumor burden was assessed by caliper measurements. Error bars indicate standard error of the mean. B) Tumor burden in individual mice within the different treatment groups at day 35. Each dot represents one mouse. Anti-PD-1 Ab indicates pembrolizumab.

FIG. 5A-5C is a series of graphs showing that tisotumab vedotin antibody-drug conjugate and MMAE free drug both drove robust A) ATP secretion and C) HMGB1 release. Activity was specific to the targeted agent (tisotumab vedotin) and free drug (MMAE). The non-targeted isotype ADC (IgG1-MMAE) did not elicit A) ATP or C) HMGB1 secretion. B) Tisotumab vedotin was active on multiple Tissue Factor positive cell lines.

FIG. 6 is images of a Western blots showing treatment of HPAFII (pancreatic carcinoma) or MDA-MB-231 (breast carcinoma) cells for 16 hours with tisotumab vedotin ADC or MMAE payloads trigger multiple ER stress pathways including phosphorylation of IRE and its down-stream target JNK as well as cleavage of ATF4. Treatment with the non-targeting H00-MMAE ADC (IgG1 MMAE) did not trigger activation of these ER stress pathways.

FIGS. 7A and 7B is a series of graphs in which Tissue Factor positive MDA-MB-231 cells killed with various agents were fed to human peripheral blood mononuclear cells (PBMCs) and immune activation assessed by increased expression of activations markers on innate CD14+ monocyte/macrophages and induction of chemokine and cytokine production. Treatment with tisotumab vedotin ADC or MMAE free drug drove monocyte/macrophage activations as monitored by A) CD86 expression by flow cytometry and B) induced release of innate chemokines including MIP10 compared to non-targeting IgG1-MMAE ADC or targeting antibody (tisotumab) alone.

FIG. 8A-8C is a series of graphs in which Tissue Factor positive MDA-MB-231 cells killed with various agents were fed to CSFE labeled human peripheral blood mononuclear cells (PBMCs) in the presence or absence of the PD1 targeting antibody pembrolizumab for 48 hours and T cells activation assessed by A) decreased CSFE fluorescent indicative of T cell proliferation and B) and C) cytokine production. Treatment with tisotumab vedotin or MMAE free drug drove T cell proliferation, which was enhanced with 2 mg/ml of pembrolizumab treatment. Production of B) IL12p70 and C) IFNγ was also increased following exposure to tisotumab vedotin and MMAE killed cell and cytokine production was increased by concomitant pembrolizumab treatment.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “tissue factor”, “TF”, “CD142”, “tissue factor antigen”, “TF antigen” and “CD142 antigen” are used interchangeably herein, and, unless specified otherwise, include any variants, isoforms and species homologs of human tissue factor which are naturally expressed by cells or are expressed on cells transfected with the tissue factor gene. In some embodiments, tissue factor comprises the amino acid sequence found under Genbank accession NP_001984.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH or VH) and a heavy chain constant region (C_{H} or C_{H}). The heavy chain constant region typically is comprised of three domains, C_{H}1, C_{H}2, and C_{H}³. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. Unless stated otherwise, the numbering of amino acid residues in the constant region is according to the EU-index as described in Kabat et al., Sequences of Proteins of Immunological Interest, 5th Ed. Public Health Service, National Institutes of Health, Bethesda, Md. (1991). An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as Clq, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to TF is substantially free of antibodies that bind specifically to antigens other than TF). An isolated antibody that binds specifically to TF can, however, have cross-reactivity to other antigens, such as TF molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-TF antibody includes a conjugate of an anti-TF antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-TF antibody is an antibody that binds to the antigen TF. In another example, an anti-PD-1 antibody is an antibody that binds to the antigen PD-1.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y EQ. 1

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate, which in the context of the present invention refers to an anti-TF antibody comprising the CDRs of tisotumab, which is coupled to monomethyl auristatin E (MMAE) as described in the present application.

The abbreviations “vc” and “val-cit” refer to the dipeptide valine-citrulline.

The abbreviation “PAB” refers to the self-immolative spacer:

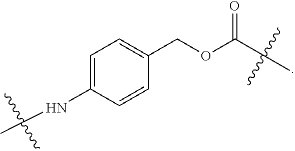

The abbreviation “MC” refers to the stretcher maleimidocaproyl:

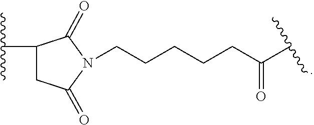 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

“Programmed Death-1” (PD-1) refers to an immunoinhibitory receptor belonging to the CD28 family. PD-1 is expressed predominantly on previously activated T-cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. In some embodiments, hPD-1 comprises the amino acid sequence found under GenBank Accession No. U64863.

“Programmed Death Ligand-1” (PD-L1) is one of two cell surface glycoprotein ligands for PD-1 (the other being PD-L2) that downregulate T-cell activation and cytokine secretion upon binding to PD-1. The term “PD-L i” as used herein includes human PD-L1 (hPD-L1), variants, isoforms, and species homologs of hPD-L1, and analogs having at least one common epitope with hPD-L1. In some embodiments, hPD-L1 comprises the amino acid sequence found under GenBank Accession No. Q9NZQ7.

“Combined positive score” or “CPS” is the ratio of the number of PD-L1 positive tumor cells and PD-L1 positive mononuclear inflammatory cells (MIC) within the tumor nests and the adjacent supporting stroma (numerator) compared to the total number of tumor cells (denominator, i.e. the number of PD-L1 positive and PD-L1 negative tumor cells).

“Tumor proportion score” or “TPS” is the percentage of viable tumor cells showing partial or complete PD-L1 membrane staining in an immunohistochemical assay at any intensity.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a cervical cancer refers to a tumor that is the result of a metastasized cervical cancer.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns”.

A therapeutically effective amount of a drug (e.g., anti-TF antibody-drug conjugate comprising MMAE and the CDRs of tisotumab or anti-PD-1 antibody comprising the CDRs of pembrolizumab) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an anti-TF antibody-drug conjugate comprising MMAE and the CDRs of tisotumab or anti-PD-1 antibody comprising the CDRs of pembrolizumab) that is lower than the usual or typical dose of the therapeutic compound when administered alone for the treatment of a hyperproliferative disease (e.g., cancer).

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 2.0 mg/kg of an anti-PD-1 antibody comprising the CDRs of pembrolizumab or an anti-TF antibody-drug conjugate comprising MMAE and the CDRs of tisotumab, one can calculate and use the appropriate amount of the anti-PD-1 antibody comprising the CDRs of pembrolizumab or anti-TF antibody-drug conjugate comprising MMAE and the CDRs of tisotumab (i.e., 120 mg) for administration to said subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., the anti-TF antibody-drug conjugate comprising MMAE and the CDRs of tisotumab and/or anti-PD-1 antibody comprising the CDRs of pembrolizumab). For example, a subject with 60 kg body weight and a subject with 100 kg body weight would receive the same dose of an antibody or an antibody-drug conjugate (e.g., 240 mg of an anti-TF antibody-drug conjugate comprising MMAE and the CDRs of tisotumab or e.g. 200 mg of an anti-PD-1 antibody comprising the CDRs of pembrolizumab).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration for the anti-TF antibody-drug conjugate comprising MMAE and the CDRs oftisotumab and/or anti-PD-1 antibody comprising the CDRs of pembrolizumab include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy (e.g., an anti-TF antibody-drug conjugate as described herein and/or an anti-PD-1 antibody as described herein) or at the beginning of administration of the therapy. The baseline value can be compared to a reference value in order to determine the reduction or improvement of a symptom of a TF-associated disease and/or PD-1 associated disease contemplated herein (e.g., breast cancer or cervical cancer). The terms “reference” or “reference value” used interchangeably herein can refer to a measurement or characterization of a symptom after administration of the therapy (e.g., an anti-TF antibody-drug conjugate as described herein and/or an anti-PD-1 antibody as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the anti-TF antibody-drug conjugate comprising MMAE and the CDRs of tisotumab or anti-PD-1 antibody comprising the CDRs of pembrolizumab is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Combination Therapy

One aspect of the invention provides anti-TF antibody-drug conjugates that binds to TF for use in the treatment of cancer wherein the antibody-drug conjugate is for administration, or to be administered in combination with an anti-PD-1 antibody or an antigen-binding fragment thereof wherein the antibody-drug conjugate comprises an anti-TF antibody or an antigen-binding fragment thereof conjugated to monomethyl auristatin E, and wherein the anti-PD-1 antibody or the antigen-binding fragment thereof inhibits PD-1 activity, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are generally defined by the Kabat numbering scheme,

and wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme. In another aspect the invention provides an anti-PD-1 antibody comprising the CDRs of pembrolizumab or an antigen-binding fragment thereof for use in the treatment of cancer wherein the anti-PD-1 antibody is for administration, or to be administered in combination with an antibody-drug conjugate that binds to TF wherein the antibody-drug conjugate comprises an anti-TF antibody or an antigen-binding fragment thereof comprising the CDRs of tisotumab conjugated to monomethyl auristatin E, and wherein the anti-PD-1 antibody or the antigen-binding fragment thereof inhibits PD-1 activity. In some embodiments, the cancer is breast cancer. In some embodiments, the breast cancer is ER+/HER2− breast cancer. In some embodiments, the breast cancer is triple negative breast cancer. In some embodiments, the cancer is cervical cancer. In some embodiments, the cervical cancer is an advanced stage cervical cancer (e.g., stage 3 cervical cancer or stage 4 cervical cancer or metastatic cervical cancer). In some embodiments, the advanced cervical cancer is a metastatic cancer. In some embodiments, the subject has relapsed, recurrent and/or metastatic cervical cancer.

A. Anti-TF Antibody

Generally, anti-TF antibodies of the disclosure bind TF, e.g., human TF, and exert cytostatic and cytotoxic effects on malignant cells, such as breast cancer cells or cervical cancer cells, wherein the anti-TF antibody or antigen binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme. Anti-TF antibodies of the disclosure comprise the CDRs of tisotumab and are preferably monoclonal, and may be multispecific, human, humanized or chimeric antibodies, single chain antibodies, Fab fragments, F(ab′) fragments, fragments produced by a Fab expression library, and TF binding fragments of any of the above. In some embodiments, the anti-TF antibodies of the disclosure comprise the CDRs of tisotumab and specifically bind TF. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-TF antibodies comprise the CDRs of tisotumab and are antigen-binding fragments (e.g., human antigen-binding fragments) as described herein and include, but are not limited to, Fab, Fab′ and F(ab′)2, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-TF antibodies or antigen-binding fragments thereof are human, murine (e.g., mouse and rat), donkey, sheep, rabbit, goat, guinea pig, camelid, horse, or chicken and comprise the CDRs of tisotumab.

The anti-TF antibodies of the present disclosure comprise the CDRs of tisotumab and may be monospecific, bispecific, trispecific or of greater multi specificity. Multispecific antibodies may be specific for different epitopes of TF or may be specific for both TF as well as for a heterologous protein. See, e.g., PCT publications WO 93/17715; WO 92/08802; WO 91/00360; WO 92/05793; Tutt, et al., 1991, J. Immunol. 147:60.69; U.S. Pat. Nos. 4,474,893; 4,714,681; 4,925,648; 5,573,920; 5,601,819; Kostelny et al., 1992, J. Immunol. 148:1547 1553.

Anti-TF antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plickthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementary determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

Numbering of amino acid residues in CDR sequences of the anti-TF antibodies of the anti-TF antibody-drug conjugate provided herein are according to the IMGT numbering scheme as described in Lefranc, M. P. et al., Dev. Comp. Immunol., 2003, 27, 55-77. CDR sequences provided herein for the anti-TF antibodies of the anti-TF antibody-drug conjugate are according to the IMGT method as described in Lefranc, M. P. et al., Dev. Comp. Immunol., 2003, 27, 55-77.

The anti-TF antibodies of the disclosure comprise the CDRs of the antibody 011. See WO 2011/157741 and WO 2010/066803. The disclosure encompasses an antibody or derivative thereof comprising a heavy or light chain variable domain, said variable domain comprising (a) a set of three CDRs, in which said set of CDRs are from monoclonal antibody 011, and (b) a set of four framework regions, in which said set of framework regions differs from the set of framework regions in monoclonal antibody 011, and in which said antibody or derivative thereof binds to TF. In some embodiments, said antibody or derivative thereof specifically binds to TF. In certain embodiments, the anti-TF antibody is 011. The antibody 011 is also known as tisotumab.

In one aspect, provided herein is an anti-TF antibody comprising a heavy chain variable region and a light chain variable region, wherein the heavy chain variable region comprises (i) CDR-H1 comprising the amino acid sequence of SEQ ID NO: 1, (ii) CDR-H2 comprising the amino acid sequence of SEQ ID NO:2, and (iii) CDR-H3 comprising the amino acid sequence of SEQ ID NO:3; and wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6, wherein the CDRs of the anti-TF antibody are defined by the IMGT numbering scheme.

An anti-TF antibody described herein may comprise any suitable framework variable domain sequence, provided that the antibody retains the ability to bind TF (e.g., human TF). As used herein, heavy chain framework regions are designated “HC-FR1-FR4,” and light chain framework regions are designated “LC-FR1-FR4.” In some embodiments, the anti-TF antibody comprises a heavy chain variable domain framework sequence of SEQ ID NO:9, 10, 11, and 12 (HC-FR1, HC-FR2, HC-FR3, and HC-FR4, respectively). In some embodiments, the anti-TF antibody comprises a light chain variable domain framework sequence of SEQ ID NO: 13, 14, 15, and 16 (LC-FR1, LC-FR2, LC-FR3, and LC-FR4, respectively).

In some embodiments of the anti-TF antibodies described herein, the heavy chain variable domain comprises the amino acid sequence of EVQLLESGGGLVQPGGSLRLSCAASGFTFSNYAMSWVRQAPGKGLEWVSSISGSGDY TYYTDSVKGRFTISRDNSKNTLYLQMNSLRAEDTAVYYCARSPWGYYLDSWGQGTL VTVSS (SEQ ID NO:7) and the light chain variable domain comprises the amino acid sequence of DIQMTQSPPSLSASAGDRVTITCRASQGISSRLAWYQQKPEKAPKSLIYAASSLQSGVP SRFSGSGSGTDFTLTISSLQPEDFATYYCQQYNSYPYTFGQGTKLEIK (SEQ ID NO:8).

In some embodiments of the anti-TF antibodies described herein, the heavy chain CDR sequences comprise the following:

a) CDR-H1 (GFTFSNYA (SEQ ID NO: 1));
b) CDR-H2 (ISGSGDYT (SEQ ID NO: 2));
and
c) CDR-H3 (ARSPWGYYLDS (SEQ ID NO: 3))

In some embodiments of the anti-TF antibodies described herein, the heavy chain FR sequences comprise the following:

a) HC-FR1
(EVQLLESGGGLVQPGGSLRLSCAAS (SEQ ID NO: 9));
b) HC-FR2
(MSWVRQAPGKGLEWVSS (SEQ ID NO: 10));
c) HC-FR3
(YYTDSVKGRFTISRDNSKNTLYLQMNSLRAEDTAVYYC (SEQ ID
NO: 11));
and
d) HC-FR4
(WGQGTLVTVSS (SEQ ID NO: 12)).

In some embodiments of the anti-TF antibodies described herein, the light chain CDR sequences comprise the following:

a) CDR-L1 (QGISSR (SEQ ID NO: 4));
b) CDR-L2 (AAS (SEQ ID NO: 5));
and
c) CDR-L3 (QQYNSYPYT (SEQ ID NO: 6))

In some embodiments of the anti-TF antibodies described herein, the light chain FR sequences comprise the following:

a) LC-FR1
(DIQMTQSPPSLSASAGDRVTITCRAS (SEQ ID NO: 13));
b) LC-FR2
(LAWYQQKPEKAPKSLIY (SEQ ID NO: 14));
c) LC-FR3;
(SLQSGVPSRFSGSGSGTDFTLTISSLQPEDFATYYC (SEQ ID NO:
15))
and
d) LC-FR4
(FGQGTKLEIK (SEQ ID NO: 16)).

In some embodiments, provided herein is an anti-TF antibody that binds to TF (e.g., human TF), wherein the antibody comprises a heavy chain variable region and a light chain variable region, wherein the antibody comprises:

(a) heavy chain variable domain comprising:
(1) an HC-FR1 comprising the amino acid sequence of SEQ ID NO:9;
(2) an CDR-H1 comprising the amino acid sequence of SEQ ID NO: 1;
(3) an HC-FR2 comprising the amino acid sequence of SEQ ID NO:10;
(4) an CDR-H2 comprising the amino acid sequence of SEQ ID NO:2;
(5) an HC-FR3 comprising the amino acid sequence of SEQ ID NO: 11;
(6) an CDR-H3 comprising the amino acid sequence of SEQ ID NO:3; and
(7) an HC-FR4 comprising the amino acid sequence of SEQ ID NO: 12,

and/or

(b) a light chain variable domain comprising:
(1) an LC-FR1 comprising the amino acid sequence of SEQ ID NO: 13;
(2) an CDR-L1 comprising the amino acid sequence of SEQ ID NO: 4;
(3) an LC-FR2 comprising the amino acid sequence of SEQ ID NO: 14;
(4) an CDR-L2 comprising the amino acid sequence of SEQ ID NO: 5;
(5) an LC-FR3 comprising the amino acid sequence of SEQ ID NO: 15;
(6) an CDR-L3 comprising the amino acid sequence of SEQ ID NO: 6; and
(7) an LC-FR4 comprising the amino acid sequence of SEQ ID NO: 16.

In one aspect, provided herein is an anti-TF antibody comprising a heavy chain variable domain comprising the amino acid sequence of SEQ ID NO:7 or comprising a light chain variable domain comprising the amino acid sequence of SEQ ID NO:8. In one aspect, provided herein is an anti-TF antibody comprising a heavy chain variable domain comprising the amino acid sequence of SEQ ID NO:7 and comprising a light chain variable domain comprising the amino acid sequence of SEQ ID NO:8. In one aspect, provided herein is an anti-TF antibody comprising the CDRs of the heavy chain variable domain comprising the amino acid sequence of SEQ ID NO:7 and comprising the CDRs of the light chain variable domain comprising the amino acid sequence of SEQ ID NO:8.

In some embodiments, provided herein is an anti-TF antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a heavy chain variable domain sequence of SEQ ID NO:7 including post-translational modifications of that sequence. In a particular embodiment, the heavy chain variable domain comprises: (a) CDR-H1 comprising the amino acid sequence of SEQ ID NO: 1, (b) CDR-H2 comprising the amino acid sequence of SEQ ID NO:2, and (c) CDR-H3 comprising the amino acid sequence of SEQ ID NO:3.

In some embodiments, provided herein is an anti-TF antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a light chain variable domain sequence of SEQ ID NO:8 including post-translational modifications of that sequence. In a particular embodiment, the light chain variable domain comprises: (a) CDR-L1 comprising the amino acid sequence of SEQ ID NO:4, (b) CDR-L2 comprising the amino acid sequence of SEQ ID NO:5, and (c) CDR-L3 comprising the amino acid sequence of SEQ ID NO:6.

In some embodiments, the anti-TF antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-TF antibody of the anti-TF antibody-drug conjugate comprises: i) a heavy chain CDR1 comprising the amino acid sequence of SEQ ID NO: 1, a heavy chain CDR2 comprising the amino acid sequence of SEQ ID NO: 2, a heavy chain CDR3 comprising the amino acid sequence of SEQ ID NO: 3; and ii) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 6, wherein the CDRs of the anti-TF antibody are defined by the IMGT numbering scheme.

In some embodiments, the anti-TF antibody of the anti-TF antibody-drug conjugate comprises: i) an amino acid sequence having at least 85% sequence identity to a heavy chain variable region comprising the amino acid sequence of SEQ ID NO: 7, and ii) an amino acid sequence having at least 85% sequence identity to a light chain variable region comprising the amino acid sequence of SEQ ID NO: 8.

In some embodiments, the anti-TF antibody of the anti-TF antibody-drug conjugate comprise the CDRs of tisotumab and is a monoclonal antibody.

In some embodiments, the anti-TF antibody of the anti-TF antibody-drug conjugate is tisotumab, which is also known as antibody 011 as described in WO 2011/157741 and WO 2010/066803.

Anti-TF antibodies of the invention comprising the CDRs of tisotumab may also be described or specified in terms of their binding affinity to TF (e.g., human TF). Preferred binding affinities include those with a dissociation constant or Kd less than 5×10⁻² M, 10⁻² M, 5×10⁻³ M, 10⁻⁴ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹¹ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to TF or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

B. Antibody-Drug Conjugate Structure

In some aspects, the anti-TF antibody-drug conjugates described herein comprise a linker between an anti-TF antibody or antigen-binding fragment thereof as described herein and monomethyl auristatin E (MMAE). In some embodiments the linker is a non-cleavable linker. In some embodiments the linker is a cleavable linker.

In some embodiments, the linker is a cleavable peptide linker comprising maleimido caproyl (MC), the dipeptide valine-citrulline (vc) and p-aminobenzylcarbamate (PAB). In some embodiments, the cleavable peptide linker has the formula: MC-vc-PAB-, wherein:

a) MC is:

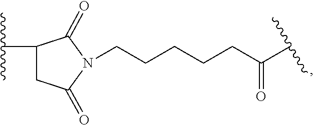 [SMILES: *C(=O)CCCCCN1C(=O)CC(*)C1=O]

b) vc is the dipeptide valine-citrulline, and

c) PAB is:

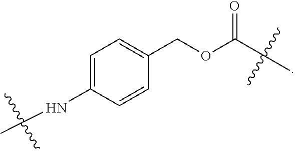

In some embodiments, the linker is a cleavable peptide linker comprising maleimido caproyl (MC). In some embodiments, the cleavable peptide linker has the formula: MC-, wherein:

a) MC is:

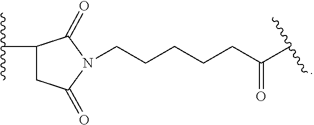 [SMILES: *C(=O)CCCCCN1C(=O)CC(*)C1=O]

In some embodiments, the linker is attached to sulphydryl residues of the anti-TF antibody or antigen-binding fragment thereof comprising the CDRs of tisotumab obtained by partial or full reduction of the anti-TF antibody or antigen-binding fragment thereof. In some embodiments, the linker is attached to sulphydryl residues of the anti-TF antibody or antigen-binding fragment thereof comprising the CDRs of tisotumab obtained by partial reduction of the anti-TF antibody or antigen-binding fragment thereof. In some embodiments, the linker is attached to sulphydryl residues of the anti-TF antibody or antigen-binding fragment thereof comprising the CDRs of tisotumab obtained by full reduction of the anti-TF antibody or antigen-binding fragment thereof.

In some aspects, the anti-TF antibody-drug conjugates described herein comprise a linker as described herein between an anti-TF antibody or antigen-binding fragment thereof as described herein and monomethyl auristatin E (MMAE). Auristatins, such as MMAE, have been shown to interfere with microtubule dynamics, GTP hydrolysis and nuclear and cellular division (See Woyke et al (2001) Antimicrob. Agents and Chemother. 45(12): 3580-3584) and have anti-cancer (See U.S. Pat. Nos. 5663149) and antifungal activity (See Pettit et al., (1998) Antimicrob. Agents and Chemother. 42: 2961-2965. MMAE, as well as suitable linkers for conjugation of MMAE to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023. The anti-TF antibody-drug conjugates described herein comprise MMAE and the CDRs of tisotumab

Monomethyl auristatin E (MMAE) has the following structure:

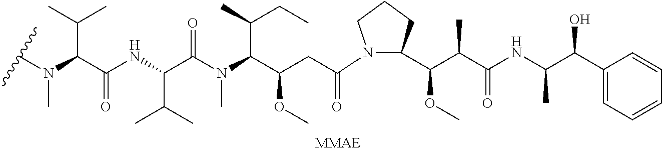 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the cleavable peptide linker has the formula: MC-vc-PAB-, and is attached to MMAE. The resulting linker-auristatin, MC-vc-PAB-MMAE is also designated vcMMAE. The vcMMAE drug linker moiety and conjugation methods are disclosed in WO2004010957, U.S. Pat. Nos. 7,659,241, 7,829,531 and 7,851,437. When vcMMAE is attached to an anti-TF antibody or antigen-binding fragment thereof comprising the CDRs of tisotumab as described herein, the resulting structure is:

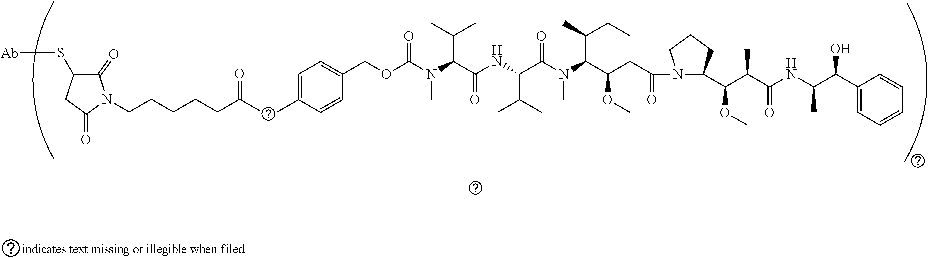

wherein p denotes a number from 1 to 8, e.g., 1, 2, 3, 4, 5, 6, 7 or 8, e.g., p may be from 3-5, S represents a sulphydryl residue of the anti-TF antibody and Ab designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the antibody-drug conjugate is tisotumab vedotin.

C. Anti-PD-1 Antibody

Generally, anti-PD-1 antibodies or antigen-binding fragments thereof of the disclosure bind to PD-1, e.g., human PD-1, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are generally defined by the Kabat numbering scheme. Anti-PD-1 antibodies of the disclosure comprise the CDRs of pembrolizumab and are preferably monoclonal, and may be multispecific, human, humanized or chimeric antibodies, single chain antibodies, Fab fragments, F(ab′) fragments, fragments produced by a Fab expression library, and PD-1 binding fragments of any of the above. In some embodiments, an anti-PD-1 antibody described herein comprises the CDRs of pembrolizumab and binds specifically to PD-1 (e.g., human PD-1). The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-1 antibodies or antigen-binding fragments thereof are human, murine (e.g., mouse and rat), donkey, sheep, rabbit, goat, guinea pig, camelid, horse, or chicken and comprise the CDRs of pembrolizumab.

The anti-PD-1 antibodies of the present disclosure comprise the CDRs of pembrolizumab and may be monospecific, bispecific, trispecific or of greater multi specificity. Multispecific antibodies may be specific for different epitopes of PD-1 or may be specific for both PD-1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-PD-1 antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plickthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementary determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

Numbering of amino acid residues in CDR sequences of the anti-PD-1 antibodies and antigen-binging fragments provided herein are generally according to the Kabat numbering scheme as described in Kabat E. A., et al., 1991, Sequences of proteins of Immunological interest, In: NIH Publication No. 91-3242, US Department of Health and Human Services, Bethesda, Md.

The anti-PD-1 antibodies of the present disclosure comprise the CDRs of the antibody pembrolizumab. See U.S. Pat. Nos. 8,354,509 and 8,900,587. The present disclosure encompasses an anti-PD-1 antibody or derivative thereof comprising a heavy or light chain variable domain, said variable domain comprising (a) a set of three CDRs, in which said set of CDRs are from the monoclonal antibody pembrolizumab, and (b) a set of four framework regions, in which said set of framework regions differs from the set of framework regions in the monoclonal antibody pembrolizumab, and in which said anti-PD-1 antibody or derivative thereof binds to PD-1. In certain embodiments, the anti-PD-1 antibody is pembrolizumab. The antibody pembrolizumab is also known as KEYTRUDA®. (Merck & Co., Inc., Kenilworth, N.J., USA).

In one aspect, provided herein is an anti-PD-1 antibody comprising a heavy chain variable region and a light chain variable region, wherein the heavy chain variable region comprises (i) CDR-H1 comprising the amino acid sequence of SEQ ID NO: 17, (ii) CDR-H2 comprising the amino acid sequence of SEQ ID NO: 18, and (iii) CDR-H3 comprising the amino acid sequence of SEQ ID NO: 19; and wherein the light chain variable region comprises (i) CDR-L1 comprising the amino acid sequence of SEQ ID NO:20, (ii) CDR-L2 comprising the amino acid sequence of SEQ ID NO:21, and (iii) CDR-L3 comprising the amino acid sequence of SEQ ID NO:22, wherein the CDRs of the anti-PD-1 antibody are generally defined by the Kabat numbering scheme.

In one embodiment, an anti-PD-1 antibody comprises a light chain variable domain comprising a framework sequence and hypervariable regions, wherein the framework sequence comprises the LC-FR1-LC-FR4 amino acid sequences of SEQ ID NO:27 (LC-FR1), SEQ ID NO:28 (LC-FR2), SEQ ID NO:29 (LC-FR3), and SEQ ID NO:30 (LC-FR4), respectively; the CDR-L1 comprises the amino acid sequence of SEQ ID NO:20; the CDR-L2 comprises the amino acid sequence of SEQ ID NO:21; and the CDR-L3 comprises the amino acid sequence of SEQ ID NO:22.

In some embodiments of the anti-PD-1 antibodies described herein, the heavy chain variable domain comprises the amino acid sequence of QVQLVQSGVEVKKPGASVKVSCKASGYTFTNYYMYWVRQAPGQGLEWMGGINPSN GGTNFNEKFKNRVTLTTDSSTTTAYMELKSLQFDDTAVYYCARRDYRFDMGFDYWG QGTTVTVSS (SEQ ID NO:31) and the light chain variable domain comprises the amino acid sequence of EIVLTQSPATLSLSPGERATLSCRASKGVSTSGYSYLHWYQQKPGQAPRLLIYLASYLE SGVPARFSGSGSGTDFTLTISSLEPEDFAVYYCQHSRDLPLTFGGGTKVEIK (SEQ ID NO:32).

In some embodiments of the anti-PD-1 antibodies described herein, the heavy chain CDR sequences comprise the following:

a) CDR-H1 (NYYMY (SEQ ID NO: 17));
b) CDR-H2 (GINPSNGGTNFNEKFKN (SEQ ID NO: 18));
and
c) CDR-H3 (RDYRFDMGFDY (SEQ ID NO: 19)).

In some embodiments of the anti-PD-1 antibodies described herein, the heavy chain FR sequences comprise the following:

a) HC-FR1
(QVQLVQSGVEVKKPGASVKVSCKASGYTFT (SEQ ID NO: 23));
b) HC-FR2
(WVRQAPGQGLEWMG (SEQ ID NO: 24));
c) HC-FR3
(RVTLTTDSSTTTAYMELKSLQFDDTAVYYCAR (SEQ ID NO:
25));
and
d) HC-FR4
(WGQGTTVTVSS (SEQ ID NO: 26)).

In some embodiments of the anti-PD-1 antibodies described herein, the light chain CDR sequences comprise the following:

a) CDR-L1 (RASKGVSTSGYSYLH (SEQ ID NO: 20));
b) CDR-L2 (LASYLES (SEQ ID NO: 21));
and
c) CDR-L3 (QHSRDLPLT (SEQ ID NO: 22)).

In some embodiments of the anti-PD-1 antibodies described herein, the light chain FR sequences comprise the following:

a) LC-FR1
(EIVLTQSPATLSLSPGERATLSC (SEQ ID NO: 27));
b) LC-FR2
(WYQQKPGQAPRLLIY (SEQ ID NO: 28));
c) LC-FR3
(GVPARFSGSGSGTDFTLTISSLEPEDFAVYYC (SEQ ID NO:
29));
and
d) LC-FR4
(FGGGTKVEIK (SEQ ID NO: 30)).

In some embodiments, provided herein is an anti-PD-1 antibody that binds to PD-1 (e.g., human PD-1), wherein the antibody comprises a heavy chain variable region and a light chain variable region, wherein the antibody comprises:

(a) heavy chain variable domain comprising:
(1) an HC-FR1 comprising the amino acid sequence of SEQ ID NO:23;
(2) an CDR-H1 comprising the amino acid sequence of SEQ ID NO: 17;
(3) an HC-FR2 comprising the amino acid sequence of SEQ ID NO:24;
(4) an CDR-H2 comprising the amino acid sequence of SEQ ID NO: 18;
(5) an HC-FR3 comprising the amino acid sequence of SEQ ID NO:25;
(6) an CDR-H3 comprising the amino acid sequence of SEQ ID NO: 19; and
(7) an HC-FR4 comprising the amino acid sequence of SEQ ID NO:26,

and/or

(b) a light chain variable domain comprising:
(1) an LC-FR1 comprising the amino acid sequence of SEQ ID NO:27;
(2) an CDR-L1 comprising the amino acid sequence of SEQ ID NO:20;
(3) an LC-FR2 comprising the amino acid sequence of SEQ ID NO:28;
(4) an CDR-L2 comprising the amino acid sequence of SEQ ID NO:21;
(5) an LC-FR3 comprising the amino acid sequence of SEQ ID NO:29;
(6) an CDR-L3 comprising the amino acid sequence of SEQ ID NO:22; and
(7) an LC-FR4 comprising the amino acid sequence of SEQ ID NO:30.

In one aspect, provided herein is an anti-PD-1 antibody comprising a heavy chain variable domain comprising the amino acid sequence of SEQ ID NO:31 or comprising a light chain variable domain comprising the amino acid sequence of SEQ ID NO:32. In one aspect, provided herein is an anti-PD-1 antibody comprising a heavy chain variable domain comprising the amino acid sequence of SEQ ID NO:31 and comprising a light chain variable domain comprising the amino acid sequence of SEQ ID NO:32. In one aspect, provided herein is an anti-PD-1 antibody comprising the CDRs of the heavy chain variable domain comprising the amino acid sequence of SEQ ID NO:31 and comprising the CDRs of the light chain variable domain comprising the amino acid sequence of SEQ ID NO:32.

In some embodiments, provided herein is an anti-PD-1 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:31. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:31 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:31. In certain embodiments, substitutions, insertions, or deletions (e.g., 1, 2, 3, 4, or 5 amino acids) occur in regions outside the CDRs (i.e., in the FRs). In some embodiments, the anti-PD-1 antibody comprises a heavy chain variable domain sequence of SEQ ID NO:31 including post-translational modifications of that sequence. In a particular embodiment, the heavy chain variable domain comprises: (a) CDR-H1 comprising the amino acid sequence of SEQ ID NO: 17, (b) CDR-H2 comprising the amino acid sequence of SEQ ID NO:18, and (c) CDR-H3 comprising the amino acid sequence of SEQ ID NO: 19.

In some embodiments, provided herein is an anti-PD-1 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:32. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:32 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:32. In certain embodiments, substitutions, insertions, or deletions (e.g., 1, 2, 3, 4, or 5 amino acids) occur in regions outside the CDR s (i.e., in the FRs). In some embodiments, the anti-PD-1 antibody comprises a light chain variable domain sequence of SEQ ID NO:32 including post-translational modifications of that sequence. In a particular embodiment, the light chain variable domain comprises: (a) CDR-L1 comprising the amino acid sequence of SEQ ID NO:20, (b) CDR-L2 comprising the amino acid sequence of SEQ ID NO:21, and (c) CDR-L3 comprising the amino acid sequence of SEQ ID NO:22.

In some embodiments, the anti-PD-1 antibody comprises a heavy chain variable domain as in any of the embodiments provided above, and a light chain variable domain as in any of the embodiments provided above. In one embodiment, the antibody comprises the heavy chain variable domain sequence of SEQ ID NO:31 and the light chain variable domain sequence of SEQ ID NO:32, including post-translational modifications of those sequences.

In some embodiments, the anti-PD-1 antibody comprises: i) a heavy chain CDR1 comprising the amino acid sequence of SEQ ID NO: 17, a heavy chain CDR2 comprising the amino acid sequence of SEQ ID NO: 18, a heavy chain CDR3 comprising the amino acid sequence of SEQ ID NO: 19; and ii) a light chain CDR1 comprising the amino acid sequence of SEQ ID NO: 20, a light chain CDR2 comprising the amino acid sequence of SEQ ID NO: 21, and a light chain CDR3 comprising the amino acid sequence of SEQ ID NO: 22, wherein the CDRs of the anti-PD-1 antibody are generally defined by the Kabat numbering scheme.

In some embodiments, the anti-PD-1 antibody comprises: i) an amino acid sequence having at least 85% sequence identity to a heavy chain variable region comprising the amino acid sequence of SEQ ID NO: 31, and ii) an amino acid sequence having at least 85% sequence identity to a light chain variable region comprising the amino acid sequence of SEQ ID NO: 32.

In some embodiments, the anti-PD-1 antibody comprises a heavy chain comprising the amino acid sequence of QVQLVQSGVEVKKPGASVKVSCKASGYTFTNYYMYWVRQAPGQGLEWMGGINPSN GGTNFNEKFKNRVTLTTDSSTTTAYMELKSLQFDDTAVYYCARRDYRFDMGFDYWG QGTTVTVSSASTKGPSVFPLAPCSRSTSESTAALGCLVKDYFPEPVTVSWNSGALTSGV HTFPAVLQSSGLYSLSSVVTVPSSSLGTKTYTCNVDHKPSNTKVDKRVESKYGPPCPPC PAPEFLGGPSVFLFPPKPKDTLMISRTPEVTCVVVDVSQEDPEVQFNWYVDGVEVHNA KTKPREEQFNSTYRVVSVLTVLHQDWLNGKEYKCKVSNKGLPSSIEKTISKAKGQPRE PQVYTLPPSQEEMTKNQVSLTCLVKGFYPSDIAVEWESNGQPENNYKTTPPVLDSDGS FFLYSRLTVDKSRWQEGNVFSCSVMHEALHNHYTQKSLSLSLG (SEQ ID NO:33) and a light chain comprising the amino acid sequence of EIVLTQSPATLSLSPGERATLSCRASKGVSTSGYSYLHWYQQKPGQAPRLLIYLASYLE SGVPARFSGSGSGTDFTLTISSLEPEDFAVYYCQHSRDLPLTFGGGTKVEIKRTVAAPS VFIFPPSDEQLKSGTASVVCLLNNFYPREAKVQWKVDNALQSGNSQESVTEQDSKDST YSLSSTLTLSKADYEKHKVYACEVTHQGLSSPVTKSFNRGEC (SEQ ID NO:34).

In some embodiments, provided herein is an anti-PD-1 antibody comprising a heavy chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:33. In certain embodiments, a heavy chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:33 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:33. In certain embodiments, substitutions, insertions, or deletions (e.g., 1, 2, 3, 4, or 5 amino acids) occur in regions outside the CDRs (i.e., in the FRs). In some embodiments, the anti-PD-1 antibody comprises a heavy chain sequence of SEQ ID NO:33 including post-translational modifications of that sequence. In a particular embodiment, the heavy chain comprises: (a) CDR-H1 comprising the amino acid sequence of SEQ ID NO: 17, (b) CDR-H2 comprising the amino acid sequence of SEQ ID NO: 18, and (c) CDR-H3 comprising the amino acid sequence of SEQ ID NO: 19.

In some embodiments, provided herein is an anti-PD-1 antibody comprising a light chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:34. In certain embodiments, a light chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:34 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:34. In certain embodiments, substitutions, insertions, or deletions (e.g., 1, 2, 3, 4, or 5 amino acids) occur in regions outside the CDRs (i.e., in the FRs). In some embodiments, the anti-PD-1 antibody comprises a light chain sequence of SEQ ID NO:34 including post-translational modifications of that sequence. In a particular embodiment, the light chain comprises: (a) CDR-L1 comprising the amino acid sequence of SEQ ID NO:20, (b) CDR-L2 comprising the amino acid sequence of SEQ ID NO:21, and (c) CDR-L3 comprising the amino acid sequence of SEQ ID NO:22.

In some embodiments, the anti-PD-1 antibody comprises the CDRs of pembrolizumab and is a monoclonal antibody.

In some embodiments, the anti-PD-1 antibody is pembrolizumab, which is also known as antibody KEYTRUDA® as described in U.S. Pat. Nos. 8,354,509 and 8,900,587.

Anti-PD-1 antibodies of the invention comprising the CDRs of pembrolizumab may also be described or specified in terms of their binding affinity to PD-1 (e.g., human PD-1). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to PD-1. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

D. Nucleic Acids, Host Cells and Methods of Production

In some aspects, also provided herein are nucleic acids encoding an anti-TF antibody or antigen-binding fragment thereof as described herein or an anti-PD-1 antibody or antigen-binding fragment thereof as described herein. Further provided herein are vectors comprising the nucleic acids encoding an anti-TF antibody or antigen-binding fragment thereof as described herein or an anti-PD-1 antibody or antigen-binding fragment thereof as described herein. Further provided herein are host cells expressing the nucleic acids encoding an anti-TF antibody or antigen-binding fragment thereof as described herein or an anti-PD-1 antibody or antigen-binding fragment thereof as described herein. Further provided herein are host cells comprising the vectors comprising the nucleic acids encoding an anti-TF antibody or antigen-binding fragment thereof as described herein or an anti-PD-1 antibody or antigen-binding fragment thereof as described herein. Methods of producing an anti-TF antibody, linker and anti-TF antibody-drug conjugate are described in U.S. Pat. No. 9,168,314.

The anti-TF antibodies described herein or anti-PD-1 antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

After isolating and purifying the anti-TF antibodies from the cell media using well known techniques in the art, they are conjugated with monomethyl auristatin E via a linker as described in U.S. Pat. No. 9,168,314.

Monoclonal anti-TF antibodies described herein or anti-PD-1 antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., J. Mol. Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody (e.g., anti-TF antibody comprising the CDRs of tisotumab or anti-PD-1 antibody comprising the CDRs of pembrolizumab) of the invention is a human antibody. Human monoclonal antibodies directed against TF or PD-1 may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and κ light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG,κ monoclonal antibodies (Lonberg, N. et al. (1994), supra; reviewed in Lonberg, N. Handbook of Experimental Pharmacology 113, 49-101 (1994), Lonberg, N. and Huszar. D., Intern. Rev. Immunol, Vol. 13 65-93 (1995) and Harding, F. and Lonberg, N. Ann, N. Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a −460 kb yeast artificial chromosome fragment of the yIgH24 chromosome. This line was designated (HCo17) 25950. The (HCo17) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain trans genes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KC05) 9272 trans gene (Fishwild eta). (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KC005 [MIK] (Balb) mice which were generated by backcrossing the KC05 strain (as described in Fishwild et (1996) Nature Biotechnology, 14:845-851) to wild-type Balb/c mice to generate mice as described in WO09097006. Using this crossing Balb/c hybrids were created for HCo12, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques. Human monoclonal or polyclonal antibodies of the invention, or antibodies of the invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the invention or antibodies of the invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### III. Binding Assays and Other Assays

In one aspect, an antibody of the invention is tested for its antigen binding activity, for example, by known methods such as Enzyme-Linked Immunosorbant Assay (ELISA), immunoblotting (e.g., Western blotting), flow cytometry (e.g., FACS™) immunohistochemistry, immunofluorescence, etc.

In another aspect, competition assays may be used to identify an antibody that competes with any one of the antibodies described herein for binding to TF (e.g., tisotumab) or PD-1 (e.g., pembrolizumab). Cross-competing antibodies can be readily identified based on their ability to cross-compete in standard TF or PD-1 binding assays such as Biacore analysis, ELISA assays or flow cytometry (See, e.g., WO 2013/173223). In certain embodiments, such a competing antibody binds to the same epitope (e.g., a linear or a conformational epitope) that is bound by any one of the antibodies disclosed herein (e.g., tisotumab orpembrolizumab,). Detailed exemplary methods for mapping an epitope to which an antibody binds are provided in Morris “Epitope Mapping Protocols,” in Methods in Molecular Biology Vol. 66 (Humana Press, Totowa, N.J., 1996).

In an exemplary competition assay, immobilized PD-1 is incubated in a solution comprising a first labeled antibody that binds to PD-1 (e.g., pembrolizumab) and a second unlabeled antibody that is being tested for its ability to compete with the first antibody for binding to PD-1. The second antibody may be present in a hybridoma supernatant. As a control, immobilized PD-1 is incubated in a solution comprising the first labeled antibody but not the second unlabeled antibody. After incubation under conditions permissive for binding of the first antibody to PD-1, excess unbound antibody is removed, and the amount of label associated with immobilized PD-1 is measured. If the amount of label associated with immobilized PD-1 is substantially reduced in the test sample relative to the control sample, then that indicates that the second antibody is competing with the first antibody for binding to PD-1. See, e.g., Harlow et al. Antibodies: A Laboratory Manual. Ch. 14 (Cold Spring Harbor Laboratory, Cold Spring Harbor, N.Y., 1988). In some embodiments, an anti-PD-1 antibody competes for binding to PD-1 with another PD-1 antibody (e.g., pembrolizumab) if the antibody blocks binding of the other antibody to PD-1 in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-PD-1 antibody does not compete for binding to PD-1 with another PD-1 antibody (e.g., pembrolizumab) if the antibody blocks binding of the other antibody to PD-1 in a competition assay by less than 20%, less than 15%, less than 10%, less than 9%, less than 8%, less than 7%, less than 6%, less than 5%, less than 4%, less than 3%, less than 2%, less than 1%. In some embodiments, the PD-1 is human PD-1.

Similar competition assays can be performed to determine if an anti-TF antibody competes with tisotumab for binding to TF. In some embodiments, an anti-TF antibody competes for binding to TF with another TF antibody (e.g., tisotumab) if the antibody blocks binding of the other antibody to TF in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-TF antibody does not compete for binding to TF with another TF antibody (e.g., tisotumab) if the antibody blocks binding of the other antibody to TF in a competition assay by less than 20%, less than 15%, less than 10%, less than 9%, less than 8%, less than 7%, less than 6%, less than 5%, less than 4%, less than 3%, less than 2%, less than 1%. In some embodiments, the TF is human TF.

### IV. Methods of Treatment

The invention provides methods for treating cancer in a subject with an anti-TF antibody-drug conjugate described herein and an anti-PD-1 antibody described herein. In one aspect, the antibody-drug conjugate is tisotumab vedotin. In one aspect, the anti-PD-1 antibody is pembrolizumab. In a particular embodiment, the subject is a human.

In another aspect the invention provides an antibody-drug conjugate that binds to TF for use in the treatment of cancer wherein the antibody-drug conjugate is for administration, or to be administered in combination with an anti-PD-1 antibody or an antigen-binding fragment thereof wherein the antibody-drug conjugate comprises an anti-TF antibody or an antigen-binding fragment thereof conjugated to monomethyl auristatin E, wherein the anti-PD-1 antibody or the antigen-binding fragment thereof inhibits PD-1 activity, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are generally defined by the Kabat numbering scheme,

and wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme.

In another aspect the invention provides an anti-PD-1 antibody or an antigen-binding fragment thereof for use in the treatment of cancer wherein the anti-PD-1 antibody is for administration, or to be administered in combination with an antibody-drug conjugate that binds to TF wherein the antibody-drug conjugate comprises an anti-TF antibody or an antigen-binding fragment thereof conjugated to monomethyl auristatin E, and wherein the anti-PD-1 antibody or the antigen-binding fragment thereof inhibits PD-1 activity, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are generally defined by the Kabat numbering scheme,

and wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme.

A. Breast Cancer

The 2014 World Cancer Report from WHO (The World health organization) reports that breast cancer is the second most common cancer worldwide, accounting for just over 1 million new cases annually. It states that in 2000 about 400,000 women died from breast cancer, representing 1.6 percent of all female deaths. The proportion of breast cancer deaths was far higher in the rich countries (2 percent of all female deaths) than in economically poor regions (0.5 percent). Thus, breast cancer is strongly related to the Western lifestyle. As developing countries succeed in achieving lifestyles similar to Europe, North America, Australia, New Zealand and Japan, they will also encounter much higher cancer rates, particularly cancers of the breast. Recent data supports this prediction and show a 20% increase in breast cancer from 2008 to 2012. (Carter D. “New global survey shows an increasing cancer burden”. Am J Nurs. 2014 March; 114(3): 17).

In some aspects, the invention provides methods for treating breast cancer in a subject with an anti-TF antibody-drug conjugate described herein and an anti-PD-1 antibody described herein. In some embodiments, the breast cancer is ER+/HER2- breast cancer. In some embodiments, the breast cancer is triple negative breast cancer. In one aspect, the antibody-drug conjugate is tisotumab vedotin. In one aspect, the anti-PD-1 antibody is pembrolizumab. In a particular embodiment, the subject is a human.

In some embodiments, at least about 0.10%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the breast cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the breast cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the breast cancer cells from the subject express PD-L1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the breast cancer cells from the subject express PD-L1. In some of any of the embodiments herein, the subject's tumor expresses PD-L1 with a tumor proportion score (TPS)≥1%. In some of embodiments herein, the subject's tumor has high PD-L1 expression (TPS≥50%). In some embodiments herein, the subject's tumor expresses PD-L1 with a combined positive score (CPS)≥1%. See US 2017/0285037. In some embodiments herein, the subject's tumor expresses PD-L1 with a combined positive score (CPS)≥10%. In some embodiments, the percentage of cells that express PD-L1 is determined using immunohistochemistry (IHC). In some embodiments, the percentage of cells that express PD-L1 is determined using flow cytometry. In some embodiments, the percentage of cells that express PD-L1 is determined using an enzyme-linked immunosorbent assay (ELISA).

In some embodiments, a tumor derived from the breast cancer comprises one or more cells that express PD-L1, PD-L2, or both PD-L1 and PD-L2.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of T-cells from the subject express PD-1. In some embodiments, the percentage of cells that express PD-1 is determined using immunohistochemistry (IHC). In some embodiments, the percentage of cells that express PD-1 is determined using flow cytometry. In some embodiments, the percentage of cells that express PD-1 is determined using an enzyme-linked immunosorbent assay (ELISA).

B. Cervical Cancer

Cervical cancer remains to be one of the leading causes of cancer-related death in women despite advances in screening, diagnosis, prevention, and treatment. It accounts for ˜4% of the total newly diagnosed cancer cases and 4% of the total cancer deaths. See Zhu et al., 2016, Drug Des. Devel. Ther. 10:1885-1895. Cervical cancer is the 7^{th} most common female cancer worldwide and the 16^{th} most common cancer in the European Union. Depending on the stage at initial presentation, cervical cancer will recur in 25-61% of women. See Tempfer et al., 2016, Oncol. Res. Treat. 39:525-533. In most cases, recurrent disease is diagnosed within 2 years of the initial treatment and may be observed in various sites. Chemotherapy is the standard treatment for these patients. See Zhu et al., 2016, Drug Des. Devel. Ther. 10:1885-1895. The median overall survival exceeds one year now, however, the five year relative survival for stage IV cervical cancer is only 15%, demonstrating the high need for improved methods of treating cervical cancer.

In some aspects, provided herein are methods for treating cervical cancer in a subject with an anti-TF antibody-drug conjugate described herein and an anti-PD-1 antibody described herein. In one aspect, the antibody-drug conjugate is tisotumab vedotin. In one aspect, the anti-PD-1 antibody is pembrolizumab. In some embodiments, the subject has not previously received prior systemic therapy for the cervical cancer. In some embodiments, chemotherapy is not considered a prior systemic therapy for the cervical cancer. In some embodiments, radiation therapy is not considered a prior systemic therapy for the cervical cancer. In some embodiments, chemotherapy in combination with radiation therapy is not considered a prior systemic therapy for the cervical cancer. In some embodiments, the subject has been previously treated with chemotherapy and/or radiation therapy. In some embodiments, the subject is not a candidate for curative therapy. In some embodiments, the curative therapy is radiotherapy and/or exenterative therapy. In some embodiments, the curative therapy is radiotherapy. In some embodiments, the curative therapy is exenterative therapy. In a particular embodiment, the subject is a human.

In some embodiments of the methods or uses or product for uses provided herein, the cervical cancer is a non-squamous cell carcinoma, an adenocarcinoma, an adenosquamous carcinoma, a squamous cell carcinoma, a small cell carcinoma, a neuroendocrine tumor, a glassy cell carcinoma or a villoglandular adenocarcinoma. In some embodiments, the cervical cancer is an adenocarcinoma, an adenosquamous carcinoma or a squamous cell carcinoma. In some embodiments, the cervical cancer is a non-squamous cell carcinoma. In some embodiments, the cervical cancer is an adenocarcinoma. In some embodiments, the cervical cancer is an adenosquamous carcinoma. In some embodiments, the cervical cancer is a squamous cell carcinoma.

In some embodiments, at least about 0.10%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the cervical cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

In some embodiments, at least about 0.10%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells from the subject express PD-L1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the cervical cancer cells from the subject express PD-L1. In some of any of the embodiments herein, the subject's tumor expresses PD-L1 with a tumor proportion score (TPS)≥1%. In some embodiments herein, the subject's tumor has high PD-L1 expression (TPS≥50%). In some embodiments herein, the subject's tumor expresses PD-L1 with a combined positive score (CPS)≥1%. See US 2017/0285037. In some embodiments herein, the subject's tumor expresses PD-L1 with a combined positive score (CPS)≥10%. In some embodiments, the percentage of cells that express PD-L1 is determined using immunohistochemistry (IHC). In some embodiments, the percentage of cells that express PD-L1 is determined using flow cytometry. In some embodiments, the percentage of cells that express PD-L1 is determined using an enzyme-linked immunosorbent assay (ELISA).

In some embodiments, a tumor derived from the cervical cancer comprises one or more cells that express PD-L1, PD-L2, or both PD-L1 and PD-L2.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of T-cells from the subject express PD-1. In some embodiments, the percentage of cells that express PD-1 is determined using immunohistochemistry (IHC). In some embodiments, the percentage of cells that express PD-1 is determined using flow cytometry. In some embodiments, the percentage of cells that express PD-1 is determined using an enzyme-linked immunosorbent assay (ELISA).

In some embodiments of the methods or uses or product for uses provided herein, the cervical cancer is a stage 0, 1, 2, 3, or 4 cervical cancer. In some embodiments, the cervical cancer is a stage 0, 1A, 1B, 2A, 2B, 3A, 3B, 4A or 4B cervical cancer. In some embodiments, the cervical cancer is staged by the International Federation of Gynecology and Obstetrics (FIGO) staging system. In some embodiments, the staging is based on clinical examination. In some embodiments, in stage 0 cervical cancer the carcinoma is confined to the surface layer (cells lining) the cervix. In some embodiments, in stage 1 cervical cancer the carcinoma has grown deeper into the cervix but has not yet spread beyond it. In some embodiments, in stage 1A cervical cancer the invasive carcinoma can be diagnosed only by microscopy and the deepest invasion is less than 5 mm and the largest extension is less than 7 mm. In some embodiments, in stage 1B cervical cancer the lesions are clinically visible and are limited to the cervix uteri. In some embodiments, in stage 2 cervical cancer the cervical carcinoma has invaded beyond the uterus, but not to the pelvic wall or to the lower third of the vagina. In some embodiments, in stage 2A cervical cancer there is no parametrial invasion. In some embodiments, in stage 2B cervical cancer there is parametrial invasion. In some embodiments, in stage 3 cervical cancer the tumor extends to the pelvic wall and/or involves the lower third of the vagina and/or causes hydronephrosis or non-functioning kidney. In some embodiments, in stage 3A cervical cancer the tumor involves the lower third of the vagina, with no extension to the pelvic wall. In some embodiments, in stage 3B cervical cancer extends to the pelvic wall and/or cause hydronephrosis or non-functioning kidney. In some embodiments, in stage 4 cervical cancer, the carcinoma has extended beyond the true pelvis or has involved the mucosa of the bladder or rectum. In some embodiments, in stage 4A cervical cancer the tumor has spread to adjacent organs. In some embodiments, in stage 4B cervical cancer the tumor has spread to distant organs. In some embodiments, the cervical cancer is an advanced stage cervical cancer. In some embodiments, the advanced stage cervical cancer is a grade 3 or grade 4 cervical cancer. In some embodiments, the advanced stage cervical cancer is metastatic cervical cancer. In some embodiments, the cervical cancer is metastatic and recurrent cervical cancer. In some embodiments, the cervical cancer is metastatic cervical cancer. In some embodiments, the cervical cancer is recurrent cervical cancer.

In some embodiments of the methods or uses or product for uses provided herein, the subject has not received prior systemic therapy for the cervical cancer. In some embodiments, chemotherapy is not considered a prior systemic therapy for the cervical cancer. In some embodiments, radiation therapy is not considered a prior systemic therapy for the cervical cancer. In some embodiments, chemotherapy in combination with radiation therapy is not considered a prior systemic therapy for the cervical cancer. In some embodiments, the subject has been previously treated with chemotherapy and/or radiation therapy. In some embodiments, the subject did not respond to the treatment with chemotherapy and radiation therapy. In some embodiments, the subject received treatment for the cervical cancer with chemotherapy and did not respond to the chemotherapy. In some embodiments, the subject received treatment for the cervical cancer with irradiation and did not respond to the irradiation. In some embodiments, the subject relapsed after treatment with chemotherapy and radiation therapy. In some embodiments, the subject received treatment for the cervical cancer with chemotherapy and relapsed after treatment with the chemotherapy. In some embodiments, the subject received treatment for the cervical cancer with irradiation and relapsed after treatment with irradiation. In some embodiments, the subject experienced disease progression after treatment with chemotherapy and/or radiation therapy. In some embodiments, the subject received treatment for the cervical cancer with chemotherapy and experienced disease progression after treatment with the chemotherapy. In some embodiments, the subject received treatment for the cervical cancer with irradiation and experienced disease progression after treatment with irradiation. In some embodiments, the subject has been previously treated for the cervical cancer with one or more therapeutic agents. In some embodiments, the subject has been previously treated with one or more therapeutic agents and did not respond to the treatment. In some embodiments, the subject has been previously treated with one or more therapeutic agents and relapsed after the treatment. In some embodiments, the subject has been previously treated with one or more therapeutic agents and experienced disease progression during treatment. In some embodiments, the one or more therapeutic agents is selected from the group consisting of a chemotherapeutic agent, pemetrexed, nab-paclitaxel, vinorelbine, bevacizumab, cisplatin, carboplatin, paclitaxel, topotecan, a combination of bevacizumab and paclitaxel, a combination of bevacizumab and cisplatin, a combination of bevacizumab and carboplatin, a combination of paclitaxel and topotecan, a combination of bevacizumab and topotecan, a combination of bevacizumab, cisplatin and paclitaxel, a combination of bevacizumab, carboplatin and paclitaxel, and a combination of bevacizumab, paclitaxel and topotecan. In some embodiments, the one or more therapeutic agents is a chemotherapeutic agent. In some embodiments, the one or more therapeutic agents is bevacizumab. In some embodiments, the one or more therapeutic agents is cisplatin, In some embodiments, the one or more therapeutic agents is carboplatin. In some embodiments, the one or more therapeutic agents is paclitaxel. In some embodiments, the one or more therapeutic agents is topotecan. In some embodiments, the one or more therapeutic agents is a combination of bevacizumab and paclitaxel. In some embodiments, the one or more therapeutic agents is a combination of bevacizumab and cisplatin. In some embodiments, the one or more therapeutic agents is a combination of bevacizumab and carboplatin. In some embodiments, the one or more therapeutic agents is a combination of paclitaxel and topotecan. In some embodiments, the one or more therapeutic agents is a combination of bevacizumab and topotecan. In some embodiments, the one or more therapeutic agents is a combination of bevacizumab, cisplatin and paclitaxel. In some embodiments, the one or more therapeutic agents is a combination of bevacizumab, carboplatin and paclitaxel. In some embodiments, the one or more therapeutic agents is a combination of bevacizumab, paclitaxel and topotecan. In some embodiments, the subject is not a candidate for curative therapy. In some embodiments, the curative therapy is radiotherapy and/or exenterative therapy. In some embodiments, the curative therapy is radiotherapy. In some embodiments, the curative therapy is exenterative therapy. In a particular embodiment, the subject is a human.

C. Routes of Administration

An anti-PD-1 antibody or antigen-binding fragment thereof described herein or anti-TF antibody-drug conjugate or antigen-binding fragment thereof described herein can be administered by any suitable route and mode. Suitable routes of administering antibodies and/or antibody-drug conjugate of the invention are well known in the art and may be selected by those of ordinary skill in the art. In one embodiment, the anti-PD-1 antibody described herein and/or anti-TF antibody-drug conjugate described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an anti-TF antibody-drug conjugate or antigen-binding fragment described herein is intravenous injection or infusion. In some embodiments, the route of administration of an anti-TF antibody-drug conjugate or antigen-binding fragment described herein is intravenous infusion. In some embodiments, the route of administration of an anti-PD-1 antibody or antigen-binding fragment described herein is intravenous injection or infusion. In some embodiments, the route of administration of an anti-PD-1 antibody or antigen-binding fragment described herein is intravenous infusion. In some embodiments, the route of administration of an anti-PD-1 antibody or antigen-binding fragment described herein is subcutaneous.

D. Dosage and Frequency of Administration

In one aspect, the invention provides for methods of treating a subject with cancer as described herein with a particular dose of an anti-TF antibody-drug conjugate or antigen-binding fragment thereof as described herein and an anti-PD-1 antibody or antigen-binding fragment thereof as described herein, wherein the subject is administered the antibody-drug conjugate or antigen-binding fragment thereof as described herein and the anti-PD-1 antibody or antigen-binding fragment thereof as described herein with particular frequencies.

In one embodiment of the methods or uses or product for uses provided herein, an anti-TF antibody-drug conjugate or antigen-binding fragment thereof as described herein is administered to the subject at a dose ranging from about 0.5 mg/kg to about 2.1 mg/kg of the subject's body weight. In certain embodiments, the dose is about 0.5 mg/kg, about 0.6 mg/kg, about 0.65 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg or about 2.1 mg/kg. In some embodiments of the methods or uses or product for uses provided herein, an anti-TF antibody-drug conjugate or antigen-binding fragment thereof as described herein is administered to the subject at a dose ranging from 0.5 mg/kg to 2.1 mg/kg of the subject's body weight. In certain embodiments, the dose is 0.5 mg/kg, 0.6 mg/kg, 0.65 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg or 2.1 mg/kg. In one embodiment, the dose is about 0.65 mg/kg. In one embodiment, the dose is 0.65 mg/kg. In one embodiment, the dose is about 0.65 mg/kg and the anti-TF antibody-drug conjugate is tisotumab vedotin. In some embodiments, the dose is 0.65 mg/kg and the anti-TF antibody-drug conjugate is tisotumab vedotin. In one embodiment, the dose is about 0.9 mg/kg. In one embodiment, the dose is 0.9 mg/kg. In one embodiment, the dose is about 0.9 mg/kg and the anti-TF antibody-drug conjugate is tisotumab vedotin. In some embodiments, the dose is 0.9 mg/kg and the anti-TF antibody-drug conjugate is tisotumab vedotin. In one embodiment, the dose is about 1.2 mg/kg. In one embodiment, the dose is 1.2 mg/kg. In one embodiment, the dose is about 1.2 mg/kg and the anti-TF antibody-drug conjugate is tisotumab vedotin. In some embodiments, the dose is 1.2 mg/kg and the anti-TF antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, or 120 mg.

In one embodiment of the methods or uses or product for uses provided herein, an anti-TF antibody-drug conjugate or antigen-binding fragment thereof as described herein is administered to the subject once about every 1 week for 3 consecutive weeks followed by about a 1 week rest period without any administration of the anti-TF antibody-drug conjugate or antigen-binding fragment thereof so that each cycle time is about 28 days including the resting period. In one embodiment of the methods or uses or product for uses provided herein, an anti-TF antibody-drug conjugate or antigen-binding fragment thereof as described herein is administered to the subject once every 1 week for 3 consecutive weeks followed by a 1 week rest period without any administration of the anti-TF antibody-drug conjugate or antigen-binding fragment thereof so that each cycle time is 28 days including the resting period. Hereby, a dosing regimen is provided where the subject to be treated is dosed with a single weekly dose for three consecutive weeks followed by a resting week. This treatment schedule may also be referred to as a “dose-dense schedule” herein and is the same as “the 4-week (28 days) cycle” and “3Q4W”. In one embodiment, an anti-TF antibody-drug conjugate or antigen-binding fragment thereof as described herein is administered to the subject on about days 1, 8, and 15 of about a 4-week cycle. In one embodiment, an anti-TF antibody-drug conjugate or antigen-binding fragment thereof as described herein is administered to the subject on days 1, 8, and 15 of a 4-week cycle. The present invention encompasses embodiments wherein the subject remains on the 3Q4W treatment cycle for at least 2, 3, 4, 5, 6, 7, 8, 9, 10, 11, 12 or more cycles. In another embodiment, the subject remains on the 3Q4W treatment cycle for between 2 and 48 cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles wherein each cycle is 28 days as described above. In some embodiments, the subject remains on the 3Q4W treatment cycle for 12 cycles or more, such as 16 cycles or more, such as 24 cycles or more, such as 36 cycles or more. In some embodiments, the 3Q4W treatment cycle is administered for no more than 3, no more than 4, no more than 5, or no more than 6 four-week treatment cycles. The number of treatment cycles suitable for any specific subject or group of subjects may be determined by a person of skill in the art, typically a physician.

In some embodiments, an anti-TF antibody-drug conjugate or antigen-binding fragment thereof as described herein is administered to the subject at a dose of about 1.2 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of about 1.2 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of about 1.2 mg/kg on about days 1, 8, and 15 of about a 4-week cycle. In some embodiments, an anti-TF antibody-drug conjugate or antigen-binding fragment thereof as described herein is administered to the subject at a dose of about 1.2 mg/kg on days 1, 8, and 15 of a 4-week cycle. In some embodiments, an anti-TF antibody-drug conjugate or antigen-binding fragment thereof as described herein is administered to the subject at a dose of 1.2 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of 1.2 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of 1.2 mg/kg on about days 1, 8, and 15 of about a 4-week cycle. In some embodiments, an anti-TF antibody-drug conjugate or antigen-binding fragment thereof as described herein is administered to the subject at a dose of 1.2 mg/kg on days 1, 8, and 15 of a 4-week cycle. In some embodiments, an anti-TF antibody-drug conjugate or antigen-binding fragment thereof as described herein is administered to the subject at a dose of about 0.9 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of about 0.9 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of about 0.9 mg/kg on about days 1, 8, and 15 of about a 4-week cycle. In some embodiments, an anti-TF antibody-drug conjugate or antigen-binding fragment thereof as described herein is administered to the subject at a dose of about 0.9 mg/kg on days 1, 8, and 15 of a 4-week cycle. In some embodiments, an anti-TF antibody-drug conjugate or antigen-binding fragment thereof as described herein is administered to the subject at a dose of 0.9 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of 0.9 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of 0.9 mg/kg on about days 1, 8, and 15 of about a 4-week cycle. In some embodiments, an anti-TF antibody-drug conjugate or antigen-binding fragment thereof as described herein is administered to the subject at a dose of 0.9 mg/kg on days 1, 8, and 15 of a 4-week cycle. In some embodiments, an anti-TF antibody-drug conjugate or antigen-binding fragment thereof as described herein is administered to the subject at a dose of about 0.65 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of about 0.65 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of about 0.65 mg/kg on about days 1, 8, and 15 of about a 4-week cycle. In some embodiments, an anti-TF antibody-drug conjugate or antigen-binding fragment thereof as described herein is administered to the subject at a dose of about 0.65 mg/kg on days 1, 8, and 15 of a 4-week cycle. In some embodiments, an anti-TF antibody-drug conjugate or antigen-binding fragment thereof as described herein is administered to the subject at a dose of 0.65 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of 0.65 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of 0.65 mg/kg on about days 1, 8, and 15 of about a 4-week cycle. In some embodiments, an anti-TF antibody-drug conjugate or antigen-binding fragment thereof as described herein is administered to the subject at a dose of 0.65 mg/kg on days 1, 8, and 15 of a 4-week cycle. In some embodiments, the dose is about 0.9 mg/kg and is administered on about days 1, 8, and 15 of about a 4-week cycle and the antibody-drug conjugate is tisotumab vedotin. In some embodiments, the dose is about 0.9 mg/kg and is administered on days 1, 8, and 15 of a 4-week cycle and the antibody-drug conjugate is tisotumab vedotin. In some embodiments, the dose is 0.9 mg/kg and is administered on about days 1, 8, and 15 of about a 4-week cycle and the antibody-drug conjugate is tisotumab vedotin. In some embodiments, the dose is 0.9 mg/kg and is administered on days 1, 8, and 15 of a 4-week cycle and the antibody-drug conjugate is tisotumab vedotin. In some embodiments, the dose is 0.9 mg/kg and is administered on about days 1, 8, and 15 of about a 4-week cycle and the antibody drug conjugate is tisotumab vedotin and the dose is decreased to 0.65 mg/kg if one or more adverse events occur. In some embodiments, the dose is 0.9 mg/kg and is administered on days 1, 8, and 15 of a 4-week cycle and the antibody drug conjugate is tisotumab vedotin and the dose is decreased to 0.65 mg/kg if one or more adverse events occur. In some embodiments, the dose is about 0.65 mg/kg and is administered on about days 1, 8, and 15 of about a 4-week cycle and the antibody-drug conjugate is tisotumab vedotin. In some embodiments, the dose is about 0.65 mg/kg and is administered on days 1, 8, and 15 of a 4-week cycle and the antibody-drug conjugate is tisotumab vedotin. In some embodiments, the dose is 0.65 mg/kg and is administered on about days 1, 8, and 15 of about a 4-week cycle and the antibody drug conjugate is tisotumab vedotin. In some embodiments, the dose is 0.65 mg/kg and is administered on days 1, 8, and 15 of a 4-week cycle and the antibody drug conjugate is tisotumab vedotin. In some embodiments, the dose is about 1.2 mg/kg and is administered on about days 1, 8, and 15 of about a 4-week cycle and the antibody-drug conjugate is tisotumab vedotin. In some embodiments, the dose is about 1.2 mg/kg and is administered on days 1, 8, and 15 of a 4-week cycle and the antibody-drug conjugate is tisotumab vedotin. In some embodiments, the dose is 1.2 mg/kg and is administered on about days 1, 8, and 15 of about a 4-week cycle and the antibody-drug conjugate is tisotumab vedotin. In some embodiments, the dose is 1.2 mg/kg and is administered on days 1, 8, and 15 of a 4-week cycle and the antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, or 120 mg.

In one embodiment of the methods or uses or product for uses provided herein, an anti-PD-1 antibody or antigen-binding fragment thereof as described herein is administered to the subject at flat dose ranging from about 50 mg to about 500 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 120 mg or a flat dose of about 140 mg or a flat dose of about 160 mg or a flat dose of about 180 mg or a flat dose of about 200 mg or a flat dose of about 220 mg or a flat dose of about 240 mg or a flat dose of about 260 mg or a flat dose of about 280 mg or a flat dose of about 300 mg or a flat dose of about 320 mg or a flat dose of about 340 mg or a flat dose of about 360 mg or a flat dose of about 380 mg or a flat dose of about 400 mg or a flat dose of about 420 mg or a flat dose of about 440 mg or a flat dose of about 460 mg or a flat dose of about 480 mg or a flat dose of about 500 mg. In some embodiments, the flat dose is about 200 mg. In some embodiments of the methods or uses or product for uses provided herein, an anti-PD-1 antibody or antigen-binding fragment thereof as described herein is administered to the subject at flat dose ranging from 50 mg to 500 mg such as at a flat dose of 50 mg or a flat dose of 60 mg or a flat dose of 70 mg or a flat dose of 80 mg or a flat dose of 90 mg or a flat dose of 100 mg or a flat dose of 120 mg or a flat dose of 140 mg or a flat dose of 160 mg or a flat dose of 180 mg or a flat dose of 200 mg or a flat dose of 220 mg or a flat dose of 240 mg or a flat dose of 260 mg or a flat dose of 280 mg or a flat dose of 300 mg or a flat dose of 320 mg or a flat dose of 340 mg or a flat dose of 360 mg or a flat dose of 380 mg or a flat dose of 400 mg or a flat dose of 420 mg or a flat dose of 440 mg or a flat dose of 460 mg or a flat dose of 480 mg or a flat dose of 500 mg. In some embodiments, the flat dose is 200 mg. In some embodiments, the flat dose is 200 mg and the anti-PD-1 antibody is pembrolizumab. In some embodiments, the flat dose is 400 mg. In some embodiments, the flat dose is 400 mg and the anti-PD-1 antibody is pembrolizumab. In some embodiments, the flat dose is about 140 mg and is administered once about every 1 week. In some embodiments, the flat dose is about 140 mg and is administered once about every 2 weeks. In some embodiments, the flat dose is about 140 mg and is administered once about every 3 weeks. In some embodiments, the flat dose is about 140 mg and is administered once about every 4 weeks. In some embodiments, the flat dose is about 160 mg and is administered once about every 1 week. In some embodiments, the flat dose is about 160 mg and is administered once about every 2 weeks. In some embodiments, the flat dose is about 160 mg and is administered once about every 3 weeks. In some embodiments, the flat dose is about 160 mg and is administered once about every 4 weeks. In some embodiments, the flat dose is about 180 mg and is administered once about every 1 week. In some embodiments, the flat dose is about 180 mg and is administered once about every 2 weeks. In some embodiments, the flat dose is about 180 mg and is administered once about every 3 weeks. In some embodiments, the flat dose is about 180 mg and is administered once about every 4 weeks. In some embodiments, the flat dose is about 200 mg and is administered once about every 1 week. In some embodiments, the flat dose is about 200 mg and is administered once about every 2 weeks. In some embodiments, the flat dose is about 200 mg and is administered once about every 3 weeks. In some embodiments, the flat dose is about 200 mg and is administered once about every 4 weeks. In some embodiments, the flat dose is about 220 mg and is administered once about every 1 week. In some embodiments, the flat dose is about 220 mg and is administered once about every 2 weeks. In some embodiments, the flat dose is about 220 mg and is administered once about every 3 weeks. In some embodiments, the flat dose is about 220 mg and is administered once about every 4 weeks. In some embodiments, the flat dose is about 240 mg and is administered once about every 1 week. In some embodiments, the dose is about 240 mg and is administered once about every 2 weeks. In some embodiments, the flat dose is about 240 mg and is administered once about every 3 weeks. In some embodiments, the flat dose is about 240 mg and is administered once about every 4 weeks. In some embodiments, the flat dose is about 260 mg and is administered once about every 1 week. In some embodiments, the flat dose is about 260 mg and is administered once about every 2 weeks. In some embodiments, the flat dose is about 260 mg and is administered once about every 3 weeks. In some embodiments, the flat dose is about 260 mg and is administered once about every 4 weeks. In some embodiments, the flat dose is about 360 mg and is administered once about every 1 week. In some embodiments, the flat dose is about 360 mg and is administered once about every 2 weeks. In some embodiments, the flat dose is about 360 mg and is administered once about every 3 weeks. In some embodiments, the flat dose is about 360 mg and is administered once about every 4 weeks. In some embodiments, the flat dose is about 360 mg and is administered once about every 5 weeks. In some embodiments, the flat dose is about 360 mg and is administered once about every 6 weeks. In some embodiments, the flat dose is about 400 mg and is administered once about every 1 week. In some embodiments, the flat dose is about 400 mg and is administered once about every 2 weeks. In some embodiments, the flat dose is about 400 mg and is administered once about every 3 weeks. In some embodiments, the flat dose is about 400 mg and is administered once about every 4 weeks. In some embodiments, the flat dose is about 400 mg and is administered once about every 5 weeks. In some embodiments, the flat dose is about 400 mg and is administered once about every 6 weeks. In some embodiments, the flat dose is about 440 mg and is administered once about every 1 week. In some embodiments, the flat dose is about 440 mg and is administered once about every 2 weeks. In some embodiments, the flat dose is about 440 mg and is administered once about every 3 weeks. In some embodiments, the flat dose is about 440 mg and is administered once about every 4 weeks. In some embodiments, the flat dose is about 440 mg and is administered once about every 5 weeks. In some embodiments, the flat dose is about 440 mg and is administered once about every 6 weeks. In some embodiments, the flat dose is 140 mg and is administered once about every 1 week. In some embodiments, the flat dose is 140 mg and is administered once about every 2 weeks. In some embodiments, the flat dose is 140 mg and is administered once about every 3 weeks. In some embodiments, the flat dose is 140 mg and is administered once about every 4 weeks. In some embodiments, the flat dose is 160 mg and is administered once about every 1 week. In some embodiments, the flat dose is 160 mg and is administered once about every 2 weeks. In some embodiments, the flat dose is 160 mg and is administered once about every 3 weeks. In some embodiments, the flat dose is 160 mg and is administered once about every 4 weeks. In some embodiments, the flat dose is 180 mg and is administered once about every 1 week. In some embodiments, the flat dose is 180 mg and is administered once about every 2 weeks. In some embodiments, the flat dose is 180 mg and is administered once about every 3 weeks. In some embodiments, the flat dose is 180 mg and is administered once about every 4 weeks. In some embodiments, the flat dose is 200 mg and is administered once about every 1 week. In some embodiments, the flat dose is 200 mg and is administered once about every 2 weeks. In some embodiments, the flat dose is 200 mg and is administered once about every 3 weeks. In some embodiments, the flat dose is 200 mg and is administered once about every 4 weeks. In some embodiments, the flat dose is 220 mg and is administered once about every 1 week. In some embodiments, the flat dose is 220 mg and is administered once about every 2 weeks. In some embodiments, the flat dose is 220 mg and is administered once about every 3 weeks. In some embodiments, the flat dose is 220 mg and is administered once about every 4 weeks. In some embodiments, the flat dose is 240 mg and is administered once about every 1 week. In some embodiments, the flat dose is 240 mg and is administered once about every 2 weeks. In some embodiments, the flat dose is 240 mg and is administered once about every 3 weeks. In some embodiments, the flat dose is 240 mg and is administered once about every 4 weeks. In some embodiments, the flat dose is 260 mg and is administered once about every 1 week. In some embodiments, the flat dose is 260 mg and is administered once about every 2 weeks. In some embodiments, the flat dose is 260 mg and is administered once about every 3 weeks. In some embodiments, the flat dose is 260 mg and is administered once about every 4 weeks. In some embodiments, the flat dose is 360 mg and is administered once about every 1 week. In some embodiments, the flat dose is 360 mg and is administered once about every 2 weeks. In some embodiments, the flat dose is 360 mg and is administered once about every 3 weeks. In some embodiments, the flat dose is 360 mg and is administered once about every 4 weeks. In some embodiments, the flat dose is 360 mg and is administered once about every 5 weeks. In some embodiments, the flat dose is 360 mg and is administered once about every 6 weeks. In some embodiments, the flat dose is 400 mg and is administered once about every 1 week. In some embodiments, the flat dose is 400 mg and is administered once about every 2 weeks. In some embodiments, the flat dose is 400 mg and is administered once about every 3 weeks. In some embodiments, the flat dose is 400 mg and is administered once about every 4 weeks. In some embodiments, the flat dose is 400 mg and is administered once about every 5 weeks. In some embodiments, the flat dose is 400 mg and is administered once about every 6 weeks. In some embodiments, the flat dose is 440 mg and is administered once about every 1 week. In some embodiments, the flat dose is 440 mg and is administered once about every 2 weeks. In some embodiments, the flat dose is 440 mg and is administered once about every 3 weeks. In some embodiments, the flat dose is 440 mg and is administered once about every 4 weeks. In some embodiments, the flat dose is 440 mg and is administered once about every 5 weeks. In some embodiments, the flat dose is 440 mg and is administered once about every 6 weeks. In some embodiments, the flat dose is 200 mg and is administered once about every 3 weeks (e.g., ±3 days). In some embodiments, the flat dose is 200 mg and is administered once every 3 weeks. In some embodiments, the flat dose is 200 mg and is administered once every 3 weeks and the antibody is pembrolizumab. In some embodiments, the flat dose is 400 mg and is administered once about every 6 weeks (e.g., ±6 days). In some embodiments, the flat dose is 400 mg and is administered once every 6 weeks. In some embodiments, the flat dose is 400 mg and is administered once every 6 weeks and the antibody is pembrolizumab. In some embodiments, the flat dose is 200 mg and is administered on about day 1 of about a 21-day cycle (e.g., ±3 days). In some embodiments, the flat dose is 200 mg and is administered on day 1 of a 21-day cycle. In some embodiments, the flat dose is 200 mg and is administered on day 1 of a 21-day cycle and the antibody is pembrolizumab. In some embodiments, the flat dose is 400 mg and is administered on about day 1 of about a 6-week cycle (e.g., ±6 days). In some embodiments, the flat dose is 400 mg and is administered on day 1 of a 6-week cycle. In some embodiments, the flat dose is 400 mg and is administered on day 1 of a 6-week cycle and the antibody is pembrolizumab.

In some embodiments, the dose of the anti-TF antibody-drug conjugate described herein is 0.65 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 0.65 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 0.65 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 0.65 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 0.65 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 0.65 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 0.65 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 0.65 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 0.65 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 0.65 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 0.65 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 0.65 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 0.7 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 0.7 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 0.7 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 0.7 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 0.7 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 0.7 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 0.7 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 0.7 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 0.7 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 0.7 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 0.7 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 0.7 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 0.8 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 0.8 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 0.8 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 0.8 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 0.8 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 0.8 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 0.8 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 0.8 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 0.8 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 0.8 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 0.8 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 0.8 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 0.9 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 0.9 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 0.9 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 0.9 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 0.9 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 0.9 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 0.9 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 0.9 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 0.9 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 0.9 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 0.9 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 0.9 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 1.0 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 1.0 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 1.0 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.0 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 1.0 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 1.0 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.0 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 1.0 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 1.0 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 1.0 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 1.0 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 1.0 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 1.1 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 1.1 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 1.1 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.1 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 1.1 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 1.1 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.1 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 1.1 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 1.1 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 1.1 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 1.1 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 1.1 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 1.2 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 1.2 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 1.2 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.2 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 1.2 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 1.2 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.2 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 1.2 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 1.2 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 1.2 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 1.2 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 1.2 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 1.3 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 1.3 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 1.3 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.3 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 1.3 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 1.3 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.3 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 1.3 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 1.3 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 1.3 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 1.3 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 1.3 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 1.4 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 1.4 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 1.4 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.4 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 1.4 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 1.4 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.4 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 1.4 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 1.4 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 1.4 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 1.4 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 1.4 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, the dose of the anti-TF antibody-drug conjugate described herein is 1.5 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 200 mg and is administered once about every 3 weeks (e.g., ±3 days). In some embodiments, the dose of the anti-TF antibody-drug conjugate described herein is 1.5 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 200 mg and is administered once every 3 weeks. In some embodiments, the dose of the anti-TF antibody-drug conjugate is 1.5 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.5 mg/kg and is administered on about days 1, 8, and 15 of about a 4-week cycle and the dose of the anti-PD-1 antibody described herein is 200 mg and is administered once about every 3 weeks (e.g., ±3 days). In some embodiments, the dose of the anti-TF antibody-drug conjugate described herein is 1.5 mg/kg and is administered on days 1, 8, and 15 of a 4-week cycle and the dose of the anti-PD-1 antibody described herein is 200 mg and is administered once every 3 weeks. In some embodiments, the dose of the anti-TF antibody-drug conjugate is 1.5 mg/kg and is administered on days 1, 8, and 15 of a 4-week cycle and the antibody-drug conjugate is tisotumab vedotin and the dose of the anti-PD-1 antibody is 200 mg and is administered once every 3 weeks and the anti-PD-1 antibody is pembrolizumab. In some embodiments, the dose of the anti-TF antibody-drug conjugate described herein is 1.5 mg/kg and is administered once about every 1 week for 3 consecutive weeks followed by about a 1 week rest period without any administration of the anti-TF antibody-drug conjugate or antigen-binding fragment thereof so that each cycle time is about 28 days including the resting period and the dose of the anti-PD-1 antibody described herein is 400 mg and is administered once about every 6 weeks (e.g., ±6 days). In some embodiments, the dose of the anti-TF antibody-drug conjugate described herein is 1.5 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the dose of the anti-PD-1 antibody described herein is 400 mg and is administered once every 6 weeks. In some embodiments, the dose of the anti-TF antibody-drug conjugate is 1.5 mg/kg and is administered once every 1 week for 3 consecutive weeks followed by a 1 week rest period without any administration of the anti-TF antibody-drug conjugate or antigen-binding fragment thereof so that each cycle time is 28 days including the resting period and the antibody-drug conjugate is tisotumab vedotin and the dose of the anti-PD-1 antibody is 400 mg and is administered once every 6 weeks and the anti-PD-1 antibody is pembrolizumab. In some embodiments, the dose of the anti-TF antibody-drug conjugate described herein is 1.5 mg/kg and is administered on about days 1, 8, and 15 of about a 4-week cycle and the dose of the anti-PD-1 antibody described herein is 400 mg and is administered once about every 6 weeks (e.g., ±6 days). In some embodiments, the dose of the anti-TF antibody-drug conjugate described herein is 1.5 mg/kg and is administered on days 1, 8, and 15 of a 4-week cycle and the dose of the anti-PD-1 antibody described herein is 400 mg and is administered once every 6 weeks. In some embodiments, the dose of the anti-TF antibody-drug conjugate is 1.5 mg/kg and is administered on days 1, 8, and 15 of a 4-week cycle and the antibody-drug conjugate is tisotumab vedotin and the dose of the anti-PD-1 antibody is 400 mg and is administered once every 6 weeks and the anti-PD-1 antibody is pembrolizumab.

In some embodiments, an anti-TF antibody-drug conjugate or antigen-binding fragment thereof as described herein and an anti-PD-1 antibody or antigen-binding fragment thereof as described herein are coadministered. In some embodiments the coadministration is simultaneous or sequential. In some embodiments, an anti-TF antibody-drug conjugate as described herein is administered simultaneously with an anti-PD-1 antibody as described herein. In some embodiments, simultaneous means that the anti-TF antibody-drug conjugate as described herein and the anti-PD-1 antibody as described herein are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate as described herein and the anti-PD-1 antibody as described herein are administered to the subject less than one hour apart, such as less than 30 minutes apart, less than 15 minutes apart, less than 10 minutes apart or less than 5 minutes apart. In some embodiments, an anti-TF antibody-drug conjugate as described herein is administered sequentially with an anti-PD-1 antibody as described herein. In some embodiments, sequential administration means that the anti-TF antibody-drug conjugate as described herein and the anti-PD-1 antibody as described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a method of treatment or use described herein further comprises the administration of one or more additional therapeutic agents. In some embodiments, the one or more additional therapeutic agents are administered simultaneously with an anti-TF antibody-drug conjugate or antigen-binding fragment thereof as described herein, such as tisotumab vedotin, and an anti-PD-1 antibody or antigen-binding fragment thereof as described herein, such as pembrolizumab. In some embodiments, the one or more additional therapeutic agents and an anti-TF antibody-drug conjugate or antigen-binding fragment thereof as described herein and an anti-PD-1 antibody or antigen-binding fragment thereof as described herein are administered sequentially.

E. Treatment Outcome

In one aspect, a method of treating cancer with an anti-TF antibody-drug conjugate or antigen-binding fragment thereof as described herein and an anti-PD-1 antibody or antigen-binding fragment thereof as described herein results in an improvement in one or more therapeutic effects in the subject after administration of the antibody-drug conjugate relative to a baseline. In some embodiments, the one or more therapeutic effects is the size of the tumor derived from the cancer (e.g., breast cancer or cervical cancer), the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with an anti-TF antibody-drug conjugate or antigen-binding fragment thereof as described herein and an anti-PD-1 antibody or antigen-binding fragment thereof as described herein may include the following criteria (RECIST Criteria 1.1):

| | Category | Criteria |
|---|---|---|
| Based on | Complete | Disappearance of all target lesions. Any pathological |
| target lesions | Response (CR) | lymph nodes must have reduction in short axis to < |
| | | 10 mm. |
| | Partial Response | ≥30% decrease in the sum of the longest diameter |
| | (PR) | (LD) of target lesions, taking as reference the |
| | | baseline sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs |
| | Disease (PD) | of target lesions, taking as reference the smallest sum |
| | | of the target LDs recorded while in trial or the |
| | | appearance of one or more new lesions. |
| Based on non- | CR | Disappearance of all non-target lesions and |
| target lesions | | normalization of tumor marker level. All lymph |
| | | nodes must be non-pathological in size (<10 mm |
| | | short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target |
| | | lesions. |

In one embodiment of the methods or uses or product for uses provided herein, the effectiveness of treatment with an anti-TF antibody-drug conjugate or antigen-binding fragment thereof described herein and an anti-PD-1 antibody or antigen-binding fragment thereof described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an anti-TF antibody-drug conjugate or antigen-binding fragment thereof described herein and an anti-PD-1 antibody or antigen-binding fragment thereof described herein is assessed by measuring the size of a tumor derived from the cancer (e.g., breast cancer or cervical cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate described herein and/or the anti-PD-1 antibody described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate described herein and/or the anti-PD-1 antibody described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%.In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In some embodiments, the size of a tumor derived from a cervical cancer is measured by pelvic examination. See Choi et al., 2008, J. Gynecol. Oncol. 19(3):205. In some embodiments, the size of a tumor derived from a breast cancer is measured by mammography, sonography or magnetic resonance imaging (MRI). See Gruber et. al., 2013, BMC Cancer. 13:328. In some embodiments, the size of the tumor derived from the cancer is reduced relative to the size of the tumor before administration of the anti-TF antibody drug conjugate described herein and the anti-PD-1 antibody described herein. In some embodiments, the size of the tumor derived from the cancer is reduced relative to the size of the tumor before administration of the anti-TF antibody drug conjugate described herein. In some embodiments, the size of the tumor derived from the cancer is reduced relative to the size of the tumor before administration of the anti-PD-1 antibody described herein.

In one embodiment of the methods or uses or product for uses provided described herein, response to treatment with an antibody-drug conjugate or antigen-binding fragment thereof described herein, such as e.g., tisotumab vedotin, and an anti-PD-1 antibody or antigen-binding fragment thereof described herein, such as e.g., pembrolizumab, promotes regression of a tumor derived from the cancer (e.g., breast cancer or cervical cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate described herein and/or anti-PD-1 antibody described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate described herein and/or anti-PD-1 antibody described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In some embodiments, regression of a tumor is determined by measuring the size of the tumor by pelvic examination. See Choi et al., 2008, J. Gynecol. Oncol. 19(3):205. In some embodiments, regression of a tumor is determined by mammography, sonography or magnetic resonance imaging (MRI). See Gruber et. al., 2013, BMC Cancer. 13:328. In some embodiments, the tumor derived from the cancer regresses relative to the size of the tumor before administration of the anti-TF antibody drug conjugate described herein and the anti-PD-1 antibody described herein. In some embodiments, the tumor derived from the cancer regresses relative to the size of the tumor before administration of the anti-TF antibody drug conjugate described herein. In some embodiments, the tumor derived from the cancer regresses relative to the size of the tumor before administration of the anti-PD-1 antibody described herein.

In one embodiment of the methods or uses or product for uses described herein, response to treatment with an anti-TF antibody-drug conjugate or antigen-binding fragment thereof described herein and an anti-PD-1 antibody or antigen-binding fragment thereof described herein is assessed by measuring the time of progression free survival after administration of the anti-TF antibody-drug conjugate described herein and/or the anti-PD-1 antibody described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate described herein and/or the anti-PD-1 antibody described herein. In some embodiments, the subject exhibits progression-free survival of at least about 6 months after administration of the anti-TF antibody-drug conjugate described herein and/or the anti-PD-1 antibody described herein. In some embodiments, the subject exhibits progression-free survival of at least about one year after administration of the anti-TF antibody-drug conjugate described herein and/or the anti-PD-1 antibody described herein. In some embodiments, the subject exhibits progression-free survival of at least about two years after administration of the anti-TF antibody-drug conjugate described herein and/or the anti-PD-1 antibody described herein. In some embodiments, the subject exhibits progression-free survival of at least about three years after administration of the anti-TF antibody-drug conjugate described herein and/or the anti-PD-1 antibody described herein. In some embodiments, the subject exhibits progression-free survival of at least about four years after administration of the anti-TF antibody-drug conjugate described herein and/or the anti-PD-1 antibody described herein. In some embodiments, the subject exhibits progression-free survival of at least about five years after administration of the anti-TF antibody-drug conjugate described herein and/or the anti-PD-1 antibody described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate described herein and/or the anti-PD-1 antibody described herein. In some embodiments, the subject exhibits progression-free survival of at least 6 months after administration of the anti-TF antibody-drug conjugate described herein and/or the anti-PD-1 antibody described herein. In some embodiments, the subject exhibits progression-free survival of at least one year after administration of the anti-TF antibody-drug conjugate described herein and/or the anti-PD-1 antibody described herein. In some embodiments, the subject exhibits progression-free survival of at least two years after administration of the anti-TF antibody-drug conjugate described herein and/or the anti-PD-1 antibody described herein. In some embodiments, the subject exhibits progression-free survival of at least three years after administration of the anti-TF antibody-drug conjugate described herein and/or the anti-PD-1 antibody described herein. In some embodiments, the subject exhibits progression-free survival of at least four years after administration of the anti-TF antibody-drug conjugate described herein and/or the anti-PD-1 antibody described herein. In some embodiments, the subject exhibits progression-free survival of at least five years after administration of the anti-TF antibody-drug conjugate described herein and/or the anti-PD-1 antibody described herein. In some embodiments, response to treatment is assessed by measuring the time of progression free survival after administration of the anti-TF antibody-drug conjugate described herein and the anti-PD-1 antibody described herein. In some embodiments, response to treatment is assessed by measuring the time of progression free survival after administration of the anti-TF antibody-drug conjugate described herein. In some embodiments, response to treatment is assessed by measuring the time of progression free survival after administration of the anti-PD-1 antibody described herein.

In one embodiment of the methods or uses or product for uses described herein, response to treatment with an anti-TF antibody-drug conjugate or antigen-binding fragment thereof described herein and an anti-PD-1 antibody or antigen-binding fragment thereof described herein is assessed by measuring the time of overall survival after administration of the anti-TF antibody-drug conjugate described herein and/or the anti-PD-1 antibody described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate described herein and/or the anti-PD-1 antibody described herein. In some embodiments, the subject exhibits overall survival of at least about 6 months after administration of the anti-TF antibody-drug conjugate described herein and/or the anti-PD-1 antibody described herein. In some embodiments, the subject exhibits overall survival of at least about one year after administration of the anti-TF antibody-drug conjugate described herein and/or the anti-PD-1 antibody described herein. In some embodiments, the subject exhibits overall survival of at least about two years after administration of the anti-TF antibody-drug conjugate described herein and/or the anti-PD-1 antibody described herein. In some embodiments, the subject exhibits overall survival of at least about three years after administration of the anti-TF antibody-drug conjugate described herein and/or the anti-PD-1 antibody described herein. In some embodiments, the subject exhibits overall survival of at least about four years after administration of the anti-TF antibody-drug conjugate described herein and/or the anti-PD-1 antibody described herein. In some embodiments, the subject exhibits overall survival of at least about five years after administration of the anti-TF antibody-drug conjugate described herein and/or the anti-PD-1 antibody described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of the anti-TF antibody-drug conjugate described herein and/or the anti-PD-1 antibody described herein. In some embodiments, the subject exhibits overall survival of at least 6 months after administration of the anti-TF antibody-drug conjugate described herein and/or the anti-PD-1 antibody described herein. In some embodiments, the subject exhibits overall survival of at least one year after administration of the anti-TF antibody-drug conjugate described herein and/or the anti-PD-1 antibody described herein. In some embodiments, the subject exhibits overall survival of at least two years after administration of the anti-TF antibody-drug conjugate described herein and/or the anti-PD-1 antibody described herein. In some embodiments, the subject exhibits overall survival of at least three years after administration of the anti-TF antibody-drug conjugate described herein and/or the anti-PD-1 antibody described herein. In some embodiments, the subject exhibits overall survival of at least four years after administration of the anti-TF antibody-drug conjugate described herein and/or the anti-PD-1 antibody described herein. In some embodiments, the subject exhibits overall survival of at least five years after administration of the anti-TF antibody-drug conjugate described herein and/or the anti-PD-1 antibody described herein. In some embodiments, response to treatment is assessed by measuring the time of overall survival after administration of the anti-TF antibody-drug conjugate described herein and the anti-PD-1 antibody described herein. In some embodiments, response to treatment is assessed by measuring the time of overall survival after administration of the anti-TF antibody-drug conjugate described herein. In some embodiments, response to treatment is assessed by measuring the time of overall survival after administration of the anti-PD-1 antibody described herein.

In one embodiment of the methods or uses or product for uses described herein, response to treatment with an anti-TF antibody-drug conjugate or antigen-binding fragment thereof described herein and an anti-PD-1 antibody or antigen-binding fragment thereof described herein is assessed by measuring the duration of response to the anti-TF antibody-drug conjugate described herein and the anti-PD-1 antibody described herein after administration of the anti-TF antibody-drug conjugate described herein and/or the anti-PD-1 antibody described herein. In some embodiments, the duration of response to the anti-TF antibody-drug conjugate described herein and the anti-PD-1 antibody described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate described herein and/or the anti-PD-1 antibody described herein. In some embodiments, the duration of response to the anti-TF antibody-drug conjugate described herein and the anti-PD-1 antibody described herein is at least about 6 months after administration of the antibody-drug conjugate described herein and/or the anti-PD-1 antibody described herein. In some embodiments, the duration of response to the anti-TF antibody-drug conjugate described herein and the anti-PD-1 antibody described herein is at least about one year after administration of the antibody-drug conjugate described herein and/or the anti-PD-1 antibody described herein. In some embodiments, the duration of response to the anti-TF antibody-drug conjugate described herein and the anti-PD-1 antibody described herein is at least about two years after administration of the antibody-drug conjugate described herein and/or the anti-PD-1 antibody described herein. In some embodiments, the duration of response to the anti-TF antibody-drug conjugate described herein and the anti-PD-1 antibody described herein is at least about three years after administration of the antibody-drug conjugate described herein and/or the anti-PD-1 antibody described herein. In some embodiments, the duration of response to the anti-TF antibody-drug conjugate described herein and the anti-PD-1 antibody described herein is at least about four years after administration of the antibody-drug conjugate described herein and/or the anti-PD-1 antibody described herein. In some embodiments, the duration of response to the anti-TF antibody-drug conjugate described herein and the anti-PD-1 antibody described herein is at least about five years after administration of the antibody-drug conjugate described herein and/or the anti-PD-1 antibody described herein. In some embodiments, the duration of response to the anti-TF antibody-drug conjugate described herein and the anti-PD-1 antibody described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate described herein and/or the anti-PD-1 antibody described herein. In some embodiments, the duration of response to the anti-TF antibody-drug conjugate described herein and the anti-PD-1 antibody described herein is at least 6 months after administration of the antibody-drug conjugate described herein and/or the anti-PD-1 antibody described herein. In some embodiments, the duration of response to the anti-TF antibody-drug conjugate described herein and the anti-PD-1 antibody described herein is at least one year after administration of the antibody-drug conjugate described herein and/or the anti-PD-1 antibody described herein. In some embodiments, the duration of response to the anti-TF antibody-drug conjugate described herein and the anti-PD-1 antibody described herein is at least two years after administration of the antibody-drug conjugate described herein and/or the anti-PD-1 antibody described herein. In some embodiments, the duration of response to the anti-TF antibody-drug conjugate described herein and the anti-PD-1 antibody described herein is at least three years after administration of the antibody-drug conjugate described herein and/or the anti-PD-1 antibody described herein. In some embodiments, the duration of response to the anti-TF antibody-drug conjugate described herein and the anti-PD-1 antibody described herein is at least four years after administration of the antibody-drug conjugate described herein and/or the anti-PD-1 antibody described herein. In some embodiments, the duration of response to the anti-TF antibody-drug conjugate described herein and the anti-PD-1 antibody described herein is at least five years after administration of the antibody-drug conjugate described herein and/or the anti-PD-1 antibody described herein. In some embodiments, the duration of response is measured after administration of the anti-TF antibody drug conjugate described herein and the anti-PD-1 antibody described herein. In some embodiments, the duration of response is measured after administration of the anti-TF antibody drug conjugate described herein. In some embodiments, the duration of response is measured after administration of the anti-PD-1 antibody described herein.

F. Adverse Events

In one aspect, a method of treating cancer (e.g., breast cancer or cervical cancer) with an anti-TF antibody-drug conjugates or antigen-binding fragments thereof described herein and an anti-PD-1 antibody or antigen-binding fragment thereof described herein results in the subject developing one or more adverse events. In some embodiments, the subject is administered an additional therapeutic agent to eliminate or reduce the severity of the adverse event. In some embodiments, the one or more adverse events the subject develops is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis, conjunctival ulceration, and/or keratitis and the additional therapeutic agent is a preservative-free lubricating eye drop, an ocular vasoconstrictor, antibiotic, a steroid eye drop, or any combination thereof. In some embodiments, the one or more adverse events is conjunctivitis, conjunctival ulceration, and keratitis and the additional therapeutic agent is a preservative-free lubricating eye drop, an ocular vasoconstrictor, antibiotic, a steroid eye drop, or any combination thereof. In some embodiments, the one or more adverse events is conjunctivitis and keratitis and the additional therapeutic agent is a preservative-free lubricating eye drop, an ocular vasoconstrictor, antibiotic, a steroid eye drop, or any combination thereof. In some embodiments, the one or more adverse events is conjunctivitis and the additional therapeutic agent is a preservative-free lubricating eye drop, an ocular vasoconstrictor, antibiotic, a steroid eye drop, or any combination thereof. In some embodiments, the one or more adverse events is keratitis and the additional therapeutic agent is a preservative-free lubricating eye drop, an ocular vasoconstrictor, antibiotic, a steroid eye drop, or any combination thereof. In some of any of the embodiments herein, the subject is administered a treatment with the additional therapeutic agent to eliminate or reduce the severity of the adverse event (e.g., conjunctivitis, conjunctival ulceration, and/or keratitis). In some embodiments, the treatment is eye cooling pads (e.g. THERA PEARL Eye Mask or similar). In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF). In some embodiments, the one or more adverse events is hyperthyroidism and the additional agent is a non-selective beta-blockers (e.g., propranolol) or thionamides. In some embodiments, the one or more adverse events is hypothyroidism and the additional agent is a thyroid replacement hormone (e.g., levothyroxine or liothyroinine).

In one aspect, the subject treated with an anti-TF antibody-drug conjugates or antigen-binding fragments thereof described herein and an anti-PD-1 antibody or antigen-binding fragment thereof described herein is at risk of developing one or more adverse events. In some embodiments, the subject is administered an additional therapeutic agent to prevent the development of the adverse event or to reduce the severity of the adverse event. In some embodiments, the one or more adverse events the subject is at risk of developing is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis, conjunctival ulceration, and/or keratitis and the additional agent is a preservative-free lubricating eye drop, an ocular vasoconstrictor, antibiotic, a steroid eye drop, or any combination thereof. In some embodiments, the one or more adverse events is conjunctivitis and keratitis and the additional agent is a preservative-free lubricating eye drop, an ocular vasoconstrictor, antibiotic, a steroid eye drop, or any combination thereof. In some embodiments, the one or more adverse events is conjunctivitis and the additional agent is a preservative-free lubricating eye drop, an ocular vasoconstrictor, antibiotic, a steroid eye drop, or any combination thereof. In some embodiments, the one or more adverse events is keratitis and the additional agent is a preservative-free lubricating eye drop, an ocular vasoconstrictor, antibiotic, a steroid eye drop, or any combination thereof. In some of any of the embodiments herein, the subject is administered a treatment with the additional therapeutic agent to prevent the development of the adverse event or to reduce the severity of the adverse event (e.g., conjunctivitis, conjunctival ulceration, and/or keratitis). In some of any of the embodiments herein, the subject is administered a treatment with the additional therapeutic agent to eliminate or reduce the severity of the adverse event (e.g., conjunctivitis, conjunctival ulceration, and/or keratitis). In some embodiments, the treatment is eye cooling pads (e.g. THERA PEARL Eye Mask or similar). In some embodiments, the one or more adverse events is a recurrent infusion related reaction and the additional agent is an antihistamine, acetaminophen and/or a corticosteroid. In some embodiments, the one or more adverse events is neutropenia and the additional agent is growth factor support (G-CSF). In some embodiments, the one or more adverse events is hyperthyroidism and the additional agent is a non-selective beta-blockers (e.g., propranolol) or thionamides. In some embodiments, the one or more adverse events is hypothyroidism and the additional agent is a thyroid replacement hormone (e.g., levothyroxine or liothyroinine).

### V. Compositions

In some aspects, also provided herein are compositions (e.g., pharmaceutical compositions and therapeutic formulations) comprising any of the anti-TF antibody-drug conjugates or antigen-binding fragments thereof described herein and/or the anti-PD-1 antibody or antigen-binding fragments thereof described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.10% to about 25% by weight or between about 10% to about 50% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

Formulations comprising an anti-TF antibody-conjugate described herein for use in methods of treatment provided herein are described in WO2015/075201. In some embodiments, an anti-TF antibody-drug conjugate described herein is in a formulation comprising the anti-TF antibody drug conjugate, histidine, sucrose, and D-mannitol, wherein the formulation has a pH of about 6.0. In some embodiments, an anti-TF antibody-drug conjugate described herein is in a formulation comprising the anti-TF antibody drug conjugate at a concentration of about 10 mg/ml, histidine at a concentration of about 30 mM, sucrose at a concentration of about 88 mM, D-mannitol at a concentration of about 165 mM, wherein the formulation has a pH of about 6.0. In some embodiments, an anti-TF antibody-drug conjugate described herein is in a formulation comprising the anti-TF antibody drug conjugate at a concentration of 10 mg/ml, histidine at a concentration of 30 mM, sucrose at a concentration of 88 mM, D-mannitol at a concentration of 165 mM, wherein the formulation has a pH of 6.0. In some embodiments, the formulation comprises tisotumab vedotin at a concentration of 10 mg/ml, histidine at a concentration of 30 mM, sucrose at a concentration of 88 mM, D-mannitol at a concentration of 165 mM, wherein the formulation has a pH of 6.0.

In some embodiments provided herein, a formulation comprising the anti-TF antibody-conjugate described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-TF antibody-drug conjugates or antigen-binding fragments thereof as described herein for use in a method of treating cervical cancer as described herein. In some aspects, provided herein are compositions comprising a population of antibody-drug conjugates, wherein the antibody-drug conjugates comprise a linker attached to MMAE, wherein the antibody-drug conjugate has the following structure:

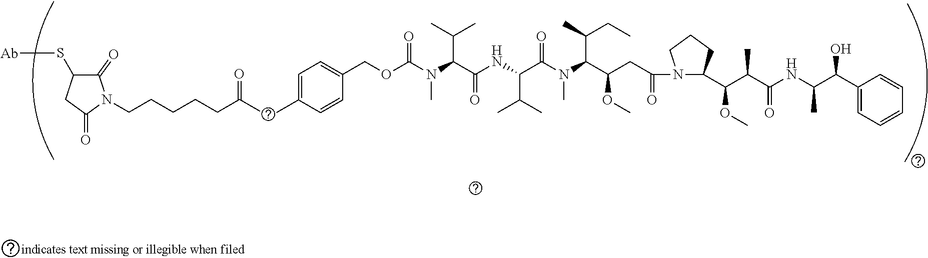

wherein p denotes a number from 1 to 8, e.g., 1, 2, 3, 4, 5, 6, 7 or 8, S represents a sulphydryl residue of the anti-TF antibody or antigen-binding fragment thereof, and Ab designates the anti-TF antibody or antigen-binding fragment thereof as described herein, such as tisotumab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for p.

In some embodiments, a composition comprising an anti-TF antibody-drug conjugate or antigen-binding fragment thereof as described herein is coadministered with a composition comprising an anti-PD-1 antibody or antigen-binding fragment thereof as described herein. In some embodiments the coadministration is simultaneous or sequential. In some embodiments, the anti-TF antibody-drug conjugate as described herein is administered simultaneously with the anti-PD-1 antibody as described herein. In some embodiments, simultaneous means that the anti-TF antibody-drug conjugate described herein and the anti-PD-1 antibody described herein are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate described herein and the anti-PD-1 antibody described herein are administered to the subject less than one hour apart, such as less than 30 minutes apart, less than 15 minutes apart, less than 10 minutes apart or less than 5 minutes apart. In some embodiments, the anti-TF antibody-drug conjugate described herein is administered sequentially with the anti-PD-1 antibody described herein. In some embodiments, sequential administration means that the anti-TF antibody-drug conjugate described herein and the anti-PD-1 antibody described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, a composition comprising an ant-TF antibody-drug conjugate as described herein and/or an anti-PD-1 antibody as described herein is coadministered with one or more therapeutic agents to eliminate or reduce the severity of one or more adverse events. In some embodiments, a composition comprising an anti-TF antibody-drug conjugate as described herein and/or an anti-PD-1 antibody as described herein is coadministered with one or more therapeutic agents to prevent the development of the adverse event or to reduce the severity of the adverse event.

In some embodiments, a composition comprising an anti-TF antibody-drug conjugate as described herein and/or anti-PD-1 antibody as described herein is coadministered with one or additional therapeutic agents. In some embodiments the coadministration is simultaneous or sequential. In some embodiments, the anti-TF antibody-drug conjugate as described herein and/or anti-PD-1 antibody as described herein is administered simultaneously with the one or more additional therapeutic agents. In some embodiments, simultaneous means that the anti-TF antibody-drug conjugate described herein and/or anti-PD-1 antibody described herein and the one or more therapeutic agents are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate described herein and/or anti-PD-1 antibody described herein and the one or more therapeutic agents are administered to the subject less than one hour apart, such as less than 30 minutes apart, less than 15 minutes apart, less than 10 minutes apart or less than 5 minutes apart. In some embodiments, the anti-TF antibody-drug conjugate described herein and/or anti-PD-1 antibody described herein is administered sequentially with the one or more additional therapeutic agents. In some embodiments, sequential administration means that the anti-TF antibody-drug conjugate described herein and/or anti-PD-1 antibody described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a composition comprising an anti-TF antibody-drug conjugate as described herein and/or anti-PD-1 antibody as described herein is coadministered with one or more therapeutic agents to eliminate or reduce the severity of one or more adverse events. In some embodiments the coadministration is simultaneous or sequential. In some embodiments, the anti-TF antibody-drug conjugate described herein and/or anti-PD-1 antibody described herein is administered simultaneously with the one or more therapeutic agents to eliminate or reduce the severity of one or more adverse events. In some embodiments, simultaneous means that the anti-TF antibody-drug conjugate described herein and/or anti-PD-1 antibody described herein and the one or more therapeutic agents to eliminate or reduce the severity of one or more adverse events are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate described herein and/or anti-PD-1 antibody described herein and the one or more therapeutic agents to eliminate or reduce the severity of one or more adverse events are administered to the subject less than one hour apart, such as less than 30 minutes apart, less than 15 minutes apart, less than 10 minutes apart or less than 5 minutes apart. In some embodiments, the anti-TF antibody-drug conjugate described herein and/or anti-PD-1 antibody described herein is administered sequentially with the one or more therapeutic agents to eliminate or reduce the severity of one or more adverse events. In some embodiments, sequential administration means that the anti-TF antibody-drug conjugate described herein and/or anti-PD-1 antibody described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-TF antibody-drug conjugate described herein and/or anti-PD-1 antibody described herein is administered prior to the one or more therapeutic agents to eliminate or reduce the severity of one or more adverse events. In some embodiments, the one or more therapeutic agents to eliminate or reduce the severity of one or more adverse events is administered prior to the anti-TF antibody-drug conjugate described herein and/or anti-PD-1 antibody described herein.

### VI. Articles of Manufacture and Kits

In another aspect, an article of manufacture or kit is provided which comprises an anti-TF antibody-drug conjugate described herein and/or an anti-PD-1 antibody described herein. The article of manufacture or kit may further comprise instructions for use of the anti-TF antibody-drug conjugate described herein and/or anti-PD-1 antibody described herein in the methods of the invention. Thus, in certain embodiments, the article of manufacture or kit comprises instructions for the use of an anti-TF antibody-drug conjugate described herein and/or an anti-PD-1 antibody described herein in methods for treating cancer (e.g., breast cancer or cervical cancer) in a subject comprising administering to the subject an effective amount of an anti-TF antibody-drug conjugate described herein and/or anti-PD-1 antibody described herein. In some embodiments, the cancer is breast cancer. In some embodiments, the breast cancer is ER+/HER2− breast cancer. In some embodiments, the breast cancer is triple negative breast cancer. In some embodiments, the cancer is cervical cancer. In some embodiments, the cervical cancer is advanced stage cervical cancer. In some embodiments, the advanced stage cervical cancer is metastatic cervical cancer. In some embodiments, the advanced stage cervical cancer is a stage 3 or stage 4 cervical cancer. In some embodiments, the cervical cancer is metastatic cancer and recurrent cancer. In some embodiments the cervical cancer is recurrent cancer. In some embodiments, the subject is not a candidate for curative therapy. In some embodiments, the subject has not received prior systemic therapy for the cervical cancer. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cancer in a subject such as breast cancer or cervical cancer described herein (e.g., advanced cervical cancer such as grade 3 or grade 4 or metastatic cervical cancer). The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-TF antibody-drug conjugate described herein is a first medicament, and which article or kit further comprises instructions on the label or package insert for treating the subject with the second medicament, in an effective amount. In some embodiments, the second medicament is an anti-PD-1 antibody as described herein. In some embodiments, the label or package insert indicates that the first and second medicaments are to be administered sequentially or simultaneously, as described herein.

The article of manufacture or kit herein optionally further comprises a container comprising a third medicament, wherein the third medicament is for eliminating or reducing the severity of one or more adverse events, wherein the anti-TF antibody-drug conjugate described herein is a first medicament, the anti-PD-1 antibody described herein is a second medicament, and which article or kit further comprises instructions on the label or package insert for treating the subject with the third medicament, in an effective amount. In some embodiments, the label or package insert indicates that the first, second and third medicaments are to be administered sequentially or simultaneously, as described herein, for example wherein the label or package insert indicates that the anti-TF antibody-drug conjugate described herein is to be administered first, followed by administration of the anti-PD-1 antibody described herein, followed by administration of the third medicament.

In some embodiments, the anti-TF antibody-drug conjugate described herein and/or anti-PD-1 antibody described herein is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

### VII. Exemplary Embodiments

Among the embodiments provided herein are:

A. Method of Treatment

1A. A method of treating cancer in a subject, the method comprising administering to the subject an antibody or an antigen-binding fragment thereof, wherein the antibody binds to Programmed Death-1 (PD-1) and inhibits PD-1 activity, and an antibody-drug conjugate that binds to tissue factor (TF), wherein the antibody-drug conjugate comprises an anti-TF antibody or an antigen-binding fragment thereof conjugated to monomethyl auristatin E, wherein the anti-PD-1 antibody or antigen-binding fragment thereof comprises a heavy chain variable region and a light chain variable region, wherein the heavy chain variable region comprises: (i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are defined by the Kabat numbering scheme;

and wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme, wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period.

2A. The method of embodiment 1A, wherein the antibody-drug conjugate is administered at a dose of about 0.65 mg/kg.
3A. The method of embodiment 1A, wherein the antibody-drug conjugate is administered at a dose of 0.65 mg/kg.
4A. The method of embodiment 1A, wherein the antibody-drug conjugate is administered at a dose of about 0.7 mg/kg.
5A. The method of embodiment 1A, wherein the antibody-drug conjugate is administered at a dose of 0.7 mg/kg.
6A. The method of embodiment 1A, wherein the antibody-drug conjugate is administered at a dose of about 0.8 mg/kg.
7A. The method of embodiment 1A, wherein the antibody-drug conjugate is administered at a dose of 0.8 mg/kg.
8A. The method of embodiment 1A, wherein the antibody-drug conjugate is administered at a dose of about 0.9 mg/kg.
9A. The method of embodiment 1A, wherein the antibody-drug conjugate is administered at a dose of 0.9 mg/kg.
10A. The method of embodiment 1A, wherein the antibody-drug conjugate is administered at a dose of about 1.0 mg/kg.
11A. The method of embodiment 1A, wherein the antibody-drug conjugate is administered at a dose of 1.0 mg/kg.
12A. The method of embodiment 1A, wherein the antibody-drug conjugate is administered at a dose of about 1.1 mg/kg.
13A. The method of embodiment 1A, wherein the antibody-drug conjugate is administered at a dose of 1.1 mg/kg.
14A. The method of embodiment 1A, wherein the antibody-drug conjugate is administered at a dose of about 1.2 mg/kg.
15A. The method of embodiment 1A, wherein the antibody-drug conjugate is administered at a dose of 1.2 mg/kg.
16A. The method of embodiment 1A, wherein the antibody-drug conjugate is administered at a dose of about 1.3 mg/kg.
17A. The method of embodiment 1A, wherein the antibody-drug conjugate is administered at a dose of 1.3 mg/kg.
18A. The method of embodiment 1A, wherein the antibody-drug conjugate is administered at a dose of about 1.4 mg/kg.
19A. The method of embodiment 1A, wherein the antibody-drug conjugate is administered at a dose of 1.4 mg/kg.
20A. The method of embodiment 1A, wherein the antibody-drug conjugate is administered at a dose of about 1.5 mg/kg.
21A. The method of embodiment 1A, wherein the antibody-drug conjugate is administered at a dose of 1.5 mg/kg.
22A. The method of any one of embodiments 1A-21A, wherein the antibody-drug conjugate is administered once every 1 week for 3 consecutive weeks followed by a 1 week rest period without any administration of the antibody-drug conjugate so that each cycle time is 28 days including the resting period.
23A. The method of any one of embodiments 1A-21A, wherein the antibody-drug conjugate is administered on about days 1, 8, and 15 of about a 4-week cycle.
24A. The method of any one of embodiments 1A-21A, wherein the antibody-drug conjugate is administered on days 1, 8, and 15 of a 4-week cycle.
25A. The method of any one of embodiments 1A-24A, wherein the anti-PD-1 antibody or antigen-binding fragment thereof is administered at a flat dose ranging from about 50 mg to about 500 mg.
26A. The method of any one of embodiments 1A-25A, wherein the anti-PD-1 antibody or antigen-binding fragment thereof is administered at a flat dose of about 200 mg.
27A. The method of any one of embodiments 1A-25A, wherein the anti-PD-1 antibody or antigen-binding fragment thereof is administered at a flat dose of 200 mg.
28A. The method of any one of embodiments 1A-25A, wherein the anti-PD-1 antibody or antigen-binding fragment thereof is administered at a flat dose of about 400 mg.
29A. The method of any one of embodiments 1A-25A, wherein the anti-PD-1 antibody or antigen-binding fragment thereof is administered at a flat dose of 400 mg.
30A. The method of any one of embodiments 1A-29A, wherein the anti-PD-1 antibody or antigen-binding fragment thereof is administered once about every 1 week, once about every 2 weeks, once about every 3 weeks, once about every 4 weeks, once about every 5 weeks, or once about every 6 weeks.
31A. The method of embodiment 30A, wherein the anti-PD-1 antibody or antigen-binding fragment thereof is administered once about every 3 weeks.
32A. The method of embodiment 30A, wherein the anti-PD-1 antibody or antigen-binding fragment thereof is administered once every 3 weeks.
33A. The method of embodiment 30A, wherein the anti-PD-1 antibody or antigen-binding fragment thereof is administered once about every 6 weeks.
34A. The method of embodiment 30A, wherein the anti-PD-1 antibody or antigen-binding fragment thereof is administered once every 6 weeks.
35A. The method of any one of embodiments 1A-30A, wherein the anti-PD-1 antibody or antigen-binding fragment thereof is administered on about day 1 of about a 21-day cycle.
36A. The method of any one of embodiments 1A-30A, wherein the anti-PD-1 antibody or antigen-binding fragment thereof is administered on day 1 of a 21-day cycle.
37A. The method of any one of embodiments 1A-30A, wherein the anti-PD-1 antibody or antigen-binding fragment thereof is administered on about day 1 of about a 6-week cycle.
38A. The method of any one of embodiments 1A-30A, wherein the anti-PD-1 antibody or antigen-binding fragment thereof is administered on day 1 of a 6-week cycle.
39A. The method of any one of embodiments 1A-38A, wherein the cancer is breast cancer.
40A. The method of embodiment 39A, wherein the breast cancer is ER+/HER2− breast cancer or triple negative breast cancer.
41A. The method of any one of embodiments 1A-38A, wherein the cancer is cervical cancer.
42A. The method of embodiment 41A, wherein the subject is not a candidate for curative therapy.
43A. The method of embodiment 42A, wherein curative therapy comprises radiotherapy and/or exenterative surgery.
44A. The method of embodiment 43A, wherein the subject has not received prior systemic therapy for the cervical cancer.
45A. The method of any one of embodiments 41A-44A, wherein the cervical cancer is a non-squamous cell carcinoma, an adenocarcinoma, an adenosquamous carcinoma or a squamous cell carcinoma.
46A. The method of embodiment 45A, wherein the cervical cancer is an adenocarcinoma.
47A. The method of embodiment 45A, wherein the cervical cancer is an adenosquamous carcinoma.
48A. The method of embodiment 45A, wherein the cervical cancer is a squamous cell carcinoma.
49A. The method of embodiment 45A, wherein the cervical cancer is a non-squamous cell carcinoma.
50A. The method of any one of embodiments 41A-49A, wherein the cervical cancer is an advanced stage cervical cancer.
51A. The method of embodiment 50A, wherein the advanced stage cervical cancer is a stage 3 or stage 4 cervical cancer.
52A. The method of embodiment 50A or 51A, wherein the advanced stage cervical cancer is metastatic cervical cancer.
53A. The method of any one of embodiments 41A-52A, wherein the cervical cancer is recurrent cervical cancer.
54A. The method of any one of embodiments 1A-53A, wherein the anti-TF antibody or antigen-binding fragment thereof of the antibody-drug conjugate is a monoclonal antibody or a monoclonal antigen-binding fragment thereof.
55A. The method of any one of embodiments 1A-54A, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
56A. The method of any one of embodiments 1A-55A, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
57A. The method of any one of embodiments 1A-56A, wherein the anti-TF antibody of the antibody-drug conjugate is tisotumab.
58A. The method of any one of embodiments 1A-57A, wherein the antibody-drug conjugate further comprises a linker between the anti-TF antibody or antigen-binding fragment thereof and the monomethyl auristatin E.
59A. The method of embodiment 58A, wherein the linker is a cleavable peptide linker.
60A. The method of embodiment 59A, wherein the cleavable peptide linker has a formula: -MC-vc-PAB-, wherein:

a) MC is:

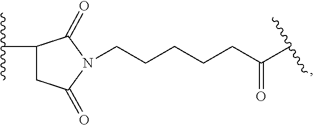 [SMILES: *C(=O)CCCCCN1C(=O)CC(*)C1=O]

b) vc is the dipeptide valine-citrulline, and

c) PAB is:

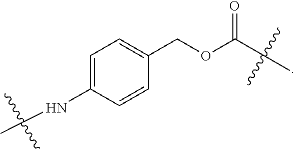

61A. The method of any one of embodiments 58A-60A, wherein the linker is attached to sulphydryl residues of the anti-TF antibody or antigen-binding fragment thereof obtained by partial reduction or full reduction of the anti-TF antibody or antigen-binding fragment thereof.
62A. The method of embodiment 61A, wherein the linker is attached to MMAE, wherein the antibody-drug conjugate has the following structure:

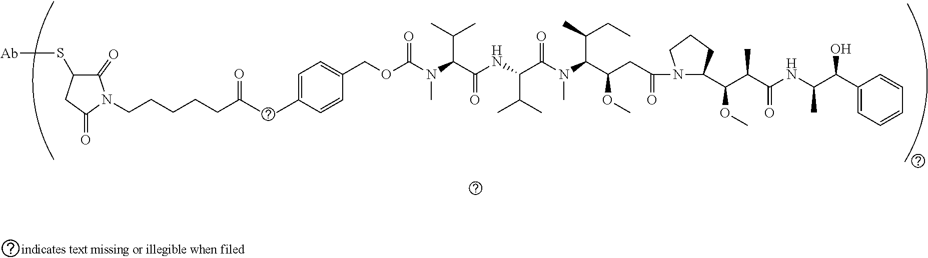

wherein p denotes a number from 1 to 8, S represents a sulphydryl residue of the anti-TF antibody, and Ab designates the anti-TF antibody or antigen-binding fragment thereof.
63A. The method of embodiment 62A, wherein the average value of p in a population of the antibody-drug conjugates is about 4.
64A. The method of any one of embodiments 1A-63A, wherein the antibody-drug conjugate is tisotumab vedotin.
65A. The method of any one of embodiments 1A-64A, wherein the route of administration for the antibody-drug conjugate is intravenous.
66A. The method of any one of embodiments 1A-65A, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO: 32.
67A. The method of any one of embodiments 1A-66A, wherein the anti-PD-1 antibody comprises a heavy chain variable region comprising the amino acid sequence of SEQ ID NO:31 and a light chain variable region comprising the amino acid sequence of SEQ ID NO:32.
68A. The method of any one of embodiments 1A-67A, wherein the anti-PD-1 antibody comprises a heavy chain comprising the amino acid sequence of SEQ ID NO:33 and a light chain comprising the amino acid sequence of SEQ ID NO:34.
69A. The method of any one of embodiments 1A-68A, wherein the anti-PD-1 antibody is pembrolizumab.
70A. The method of any one of embodiments 1A-69A, wherein the route of administration for the anti-PD-1 antibody or antigen-binding fragment thereof is intravenous or subcutaneous.
71A. The method any one of embodiments 1A-69A, wherein the route of administration for the anti-PD-1 antibody or antigen-binding fragment thereof is intravenous.
72A. The method of any one of embodiments 1A-69A, wherein the route of administration for the anti-PD-1 antibody or antigen-binding fragment thereof is subcutaneous.
73A. The method of any one of embodiments 1A-72A, wherein the anti-PD-1 antibody or antigen-binding fragment thereof and the antibody-drug conjugate are administered sequentially.
74A. The method of any one of embodiments 1A-72A, wherein the anti-PD-1 antibody or antigen-binding fragment thereof and the antibody-drug conjugate are administered simultaneously.
75A. The method of any one of embodiments 1A-74A, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
76A. The method of any one of embodiments 1A-75A, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
77A. The method of any one of embodiments 1A-76A, wherein the subject has a tumor that expresses PD-L1 (TPS≥1).
78A. The method of any one of embodiments 1A-77A, wherein the subject has a tumor that has high PD-L1 expression (TPS≥50).
79A. The method of any one of embodiments 1A-76A, wherein the subject has a tumor that expresses PD-L1 (CPS≥1).
80A. The method of any one of embodiments 1A-79A, wherein a tumor derived from the cancer comprises one or more cells that express PD-L1, PD-L2, or both PD-L1 and PD-L2.
81A. The method of any one of embodiments 1A-80A, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
82A. The method of any one of embodiments 1A-81A, wherein one or more therapeutic effects in the subject is improved after administration of the antibody-drug conjugate and the anti-PD-1 antibody or antigen-binding fragment thereof relative to a baseline.
83A. The method of embodiment 82A, wherein the one or more therapeutic effects is selected from the group consisting of: size of a tumor derived from the cancer, objective response rate, duration of response, time to response, progression free survival, and overall survival.
84A. The method of any one of embodiments 1A-83A, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
85A. The method of any one of embodiments 1A-84A, wherein the objective response rate is at least about 20%, at least about 25%, at least about 30%, at least about 35%, at least about 40%, at least about 45%, at least about 50%, at least about 60%, at least about 70%, or at least about 80%.
86A. The method of any one of embodiments 1A-85A, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
87A. The method of any one of embodiments 1A-86A, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
88A. The method of any one of embodiments 1A-87A, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
89A. The method of any one of embodiments 1A-88A, wherein the subject has one or more adverse events and is further administered an additional therapeutic agent to eliminate or reduce the severity of the one or more adverse events.
90A. The method of any one of embodiments 1A-89A, wherein the subject is at risk of developing one or more adverse events and is further administered an additional therapeutic agent to prevent or reduce the severity of the one or more adverse events.
91A. The method of any one of embodiments 87A-88A, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
92A. The method of any one of embodiments 89A-91A, wherein the one or more adverse events is a grade 3 or greater adverse event.
93A. The method of any one of embodiments 89A-91A, wherein the one or more adverse events is a serious adverse event.
94A. The method of any one of embodiments 89A-91A, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
95A. The method of any one of embodiments 1A-94A, wherein the subject is a human.
96A. The method of any one of embodiments 1A-95A, wherein the antibody-drug conjugate is in a pharmaceutical composition comprising the antibody-drug conjugate and a pharmaceutical acceptable carrier.
97A. The method of any one of embodiments 1A-96A, wherein the anti-PD-1 antibody or antigen-binding fragment thereof is in a pharmaceutical composition comprising the anti-PD-1 antibody or antigen-binding fragment thereof and a pharmaceutical acceptable carrier.
98A. A kit comprising:
(a) a dosage ranging from about 50 mg to about 500 mg of an antibody or an antigen-binding fragment thereof, wherein the antibody binds to Programmed Death-1 (PD-1) and inhibits PD-1 activity, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are defined by the Kabat numbering scheme;

(b) a dosage ranging from about 5 mg/kg to about 200 mg/kg of an antibody-drug conjugate that binds to tissue factor (TF), wherein the antibody-drug conjugate comprises an anti-TF antibody or an antigen-binding fragment thereof conjugated to monomethyl auristatin E, wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme; and

(c) instructions for use of the anti-PD-1 antibody or antigen-binding fragment thereof and the antibody-drug conjugate according to the method of any one of embodiments 1A-97A.
99A. The kit of embodiment 98A, wherein the anti-PD-1 antibody or antigen-binding fragment thereof is pembrolizumab.
100A. The kit of embodiment 99A, wherein the dose of the pembrolizumab is 200 mg.
101A. The kit of embodiment 99A, wherein the dose of the pembrolizumab is 200 mg.
102A. The kit of any one of embodiments 98A-101A, wherein the antibody-drug conjugate is tisotumab vedotin.

B. Antibody-Drug Conjugate for Use

1B. An antibody-drug conjugate that binds to TF for use in the treatment of cancer in a subject, wherein the antibody-drug conjugate is for administration, or to be administered in combination with an anti-PD-1 antibody or an antigen-binding fragment thereof, wherein the antibody-drug conjugate comprises an anti-TF antibody or an antigen-binding fragment thereof conjugated to monomethyl auristatin E, wherein the anti-PD-1 antibody or the antigen-binding fragment thereof inhibits PD-1 activity, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are defined by the Kabat numbering scheme;

and wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO: 5; and

(iii) a CDR-L3 comprising the amino acid sequence of SEQ ID NO:6, wherein the CDRs of the anti-TF antibody or antigen-binding fragment thereof are defined by the IMGT numbering scheme, wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period.

2B. The antibody-drug conjugate for use of embodiment 1B, wherein the antibody-drug conjugate is administered at a dose of about 0.65 mg/kg.
3B. The antibody-drug conjugate for use of embodiment 1B, wherein the antibody-drug conjugate is administered at a dose of 0.65 mg/kg.
4B. The antibody-drug conjugate for use of embodiment 1B, wherein the antibody-drug conjugate is administered at a dose of about 0.7 mg/kg.
5B. The antibody-drug conjugate for use of embodiment 1B, wherein the antibody-drug conjugate is administered at a dose of 0.7 mg/kg.
6B. The antibody-drug conjugate for use of embodiment 1B, wherein the antibody-drug conjugate is administered at a dose of about 0.8 mg/kg.
7B. The antibody-drug conjugate for use of embodiment 1B, wherein the antibody-drug conjugate is administered at a dose of 0.8 mg/kg.
8B. The antibody-drug conjugate for use of embodiment 1B, wherein the antibody-drug conjugate is administered at a dose of about 0.9 mg/kg.
9B. The antibody-drug conjugate for use of embodiment 1B, wherein the antibody-drug conjugate is administered at a dose of 0.9 mg/kg.
10B. The antibody-drug conjugate for use of embodiment 1B, wherein the antibody-drug conjugate is administered at a dose of about 1.0 mg/kg.
11B. The antibody-drug conjugate for use of embodiment 1B, wherein the antibody-drug conjugate is administered at a dose of 1.0 mg/kg.
12B. The antibody-drug conjugate for use of embodiment 1B, wherein the antibody-drug conjugate is administered at a dose of about 1.1 mg/kg.
13B. The antibody-drug conjugate for use of embodiment 1B, wherein the antibody-drug conjugate is administered at a dose of 1.1 mg/kg.
14B. The antibody-drug conjugate for use of embodiment 1B, wherein the antibody-drug conjugate is administered at a dose of about 1.2 mg/kg.
15B. The antibody-drug conjugate for use of embodiment 1B, wherein the antibody-drug conjugate is administered at a dose of 1.2 mg/kg.
16B. The antibody-drug conjugate for use of embodiment 1B, wherein the antibody-drug conjugate is administered at a dose of about 1.3 mg/kg.
17B. The antibody-drug conjugate for use of embodiment 1B, wherein the antibody-drug conjugate is administered at a dose of 1.3 mg/kg.
18B. The antibody-drug conjugate for use of embodiment 1B, wherein the antibody-drug conjugate is administered at a dose of about 1.4 mg/kg.
19B. The antibody-drug conjugate for use of embodiment 1B, wherein the antibody-drug conjugate is administered at a dose of 1.4 mg/kg.
20B. The antibody-drug conjugate for use of embodiment 1B, wherein the antibody-drug conjugate is administered at a dose of about 1.5 mg/kg.
21B. The antibody-drug conjugate for use of embodiment 1B, wherein the antibody-drug conjugate is administered at a dose of 1.5 mg/kg.
22B. The antibody-drug conjugate for use of any one of embodiments 1B-21B, wherein the antibody-drug conjugate is administered once every 1 week for 3 consecutive weeks followed by a 1 week rest period without any administration of the antibody-drug conjugate so that each cycle time is 28 days including the resting period.
23B. The antibody-drug conjugate for use of any one of embodiments 1B-21B, wherein the antibody-drug conjugate is administered on about days 1, 8, and 15 of about a 4-week cycle.
24B. The antibody-drug conjugate for use of any one of embodiments 1B-21B, wherein the antibody-drug conjugate is administered on days 1, 8, and 15 of a 4-week cycle.
25B. The antibody-drug conjugate for use of any one of embodiments 1B-24B, wherein the anti-PD-1 antibody or antigen-binding fragment thereof is administered at a flat dose ranging from about 50 mg to about 500 mg.
26B. The antibody-drug conjugate for use of any one of embodiments 1B-25B, wherein the anti-PD-1 antibody or antigen-binding fragment thereof is administered at a flat dose of about 200 mg.
27B. The antibody-drug conjugate for use of any one of embodiments 1B-25B, wherein the anti-PD-1 antibody or antigen-binding fragment thereof is administered at a flat dose of 200 mg.
28B. The antibody-drug conjugate for use of any one of embodiments 1B-25B, wherein the anti-PD-1 antibody or antigen-binding fragment thereof is administered at a flat dose of about 400 mg.
29B. The antibody-drug conjugate for use of any one of embodiments 1B-25B, wherein the anti-PD-1 antibody or antigen-binding fragment thereof is administered at a flat dose of 400 mg.
30B. The antibody-drug conjugate for use of any one of embodiments 1B-29B, wherein the anti-PD-1 antibody or antigen-binding fragment thereof is administered once about every 1 week, once about every 2 weeks, once about every 3 weeks, once about every 4 weeks, once about every 5 weeks, or once about every 6 weeks.
31B. The antibody-drug conjugate for use of embodiment 30B, wherein the anti-PD-1 antibody or antigen-binding fragment thereof is administered once about every 3 weeks.
32B. The antibody-drug conjugate for use of embodiment 30B, wherein the anti-PD-1 antibody or antigen-binding fragment thereof is administered once every 3 weeks.
33B. The antibody-drug conjugate for use of embodiment 30B, wherein the anti-PD-1 antibody or antigen-binding fragment thereof is administered once about every 6 weeks.
34B. The antibody-drug conjugate for use of embodiment 30B, wherein the anti-PD-1 antibody or antigen-binding fragment thereof is administered once every 6 weeks.
35B. The antibody-drug conjugate for use of any one of embodiments 1B-30B, wherein the anti-PD-1 antibody or antigen-binding fragment thereof is administered on about day 1 of about a 21-day cycle.
36B. The antibody-drug conjugate for use of any one of embodiments 1B-30B, wherein the anti-PD-1 antibody or antigen-binding fragment thereof is administered on day 1 of a 21-day cycle.
37B. The antibody-drug conjugate for use of any one of embodiments 1B-30B, wherein the anti-PD-1 antibody or antigen-binding fragment thereof is administered on about day 1 of about a 6-week cycle.
38B. The antibody-drug conjugate for use of any one of embodiments 1B-30B, wherein the anti-PD-1 antibody or antigen-binding fragment thereof is administered on day 1 of a 6-week cycle.
39B. The antibody-drug conjugate for use of any one of embodiments 1B-38B, wherein the cancer is breast cancer.
40B. The antibody-drug conjugate for use of embodiment 39B, wherein the breast cancer is ER+/HER2− breast cancer or triple negative breast cancer.
41B. The antibody-drug conjugate for use of any one of embodiments 1B-38B, wherein the cancer is cervical cancer.
42B. The antibody-drug conjugate for use of embodiment 41B, wherein the subject is not a candidate for curative therapy.
43B. The antibody-drug conjugate for use of embodiment 42B, wherein curative therapy comprises radiotherapy and/or exenterative surgery.
44B. The antibody-drug conjugate for use of embodiment 43B, wherein the subject has not received prior systemic therapy for the cervical cancer.
45B. The antibody-drug conjugate for use of any one of embodiments 41B-44B, wherein the cervical cancer is a non-squamous cell carcinoma, an adenocarcinoma, an adenosquamous carcinoma or a squamous cell carcinoma.
46B. The antibody-drug conjugate for use of embodiment 45B, wherein the cervical cancer is an adenocarcinoma.
47B. The antibody-drug conjugate for use of embodiment 45B, wherein the cervical cancer is an adenosquamous carcinoma.
48B. The antibody-drug conjugate for use of embodiment 45B, wherein the cervical cancer is a squamous cell carcinoma.
49B. The antibody-drug conjugate for use of embodiment 45B, wherein the cervical cancer is a non-squamous cell carcinoma.
50B. The antibody-drug conjugate for use of any one of embodiments 41B-49B, wherein the cervical cancer is an advanced stage cervical cancer.
51B. The antibody-drug conjugate for use of embodiment 50B, wherein the advanced stage cervical cancer is a stage 3 or stage 4 cervical cancer.
52B. The antibody-drug conjugate for use of embodiment 50B or 51B, wherein the advanced stage cervical cancer is metastatic cervical cancer.
53B. The antibody-drug conjugate for use of any one of embodiments 41B-52B, wherein the cervical cancer is recurrent cervical cancer.
54B. The antibody-drug conjugate for use of any one of embodiments 1B-53B, wherein the anti-TF antibody or antigen-binding fragment thereof of the antibody-drug conjugate is a monoclonal antibody or a monoclonal antigen-binding fragment thereof.
55B. The antibody-drug conjugate for use of any one of embodiments 1B-54B, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
56B. The antibody-drug conjugate for use of any one of embodiments 1B-55B, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO: 8.
57B. The antibody-drug conjugate for use of any one of embodiments 1B-56B, wherein the anti-TF antibody of the antibody-drug conjugate is tisotumab.
58B. The antibody-drug conjugate for use of any one of embodiments 1B-57B, wherein the antibody-drug conjugate further comprises a linker between the anti-TF antibody or antigen-binding fragment thereof and the monomethyl auristatin E.
59B. The antibody-drug conjugate for use of embodiment 58B, wherein the linker is a cleavable peptide linker.
60B. The antibody-drug conjugate for use of embodiment 59B, wherein the cleavable peptide linker has a formula: -MC-vc-PAB-, wherein:

a) MC is:

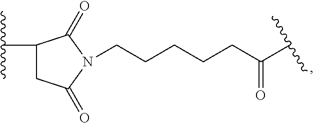 [SMILES: *C(=O)CCCCCN1C(=O)CC(*)C1=O]

b) vc is the dipeptide valine-citrulline, and

c) PAB is:

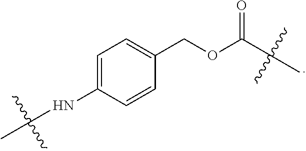 [SMILES: *Nc1ccc(COC(*)=O)cc1]

61B. The antibody-drug conjugate for use of any one of embodiments 58B-60B, wherein the linker is attached to sulphydryl residues of the anti-TF antibody or antigen-binding fragment thereof obtained by partial reduction or full reduction of the anti-TF antibody or antigen-binding fragment thereof.
62B. The antibody-drug conjugate for use of embodiment 61B, wherein the linker is attached to MMAE, wherein the antibody-drug conjugate has the following structure:

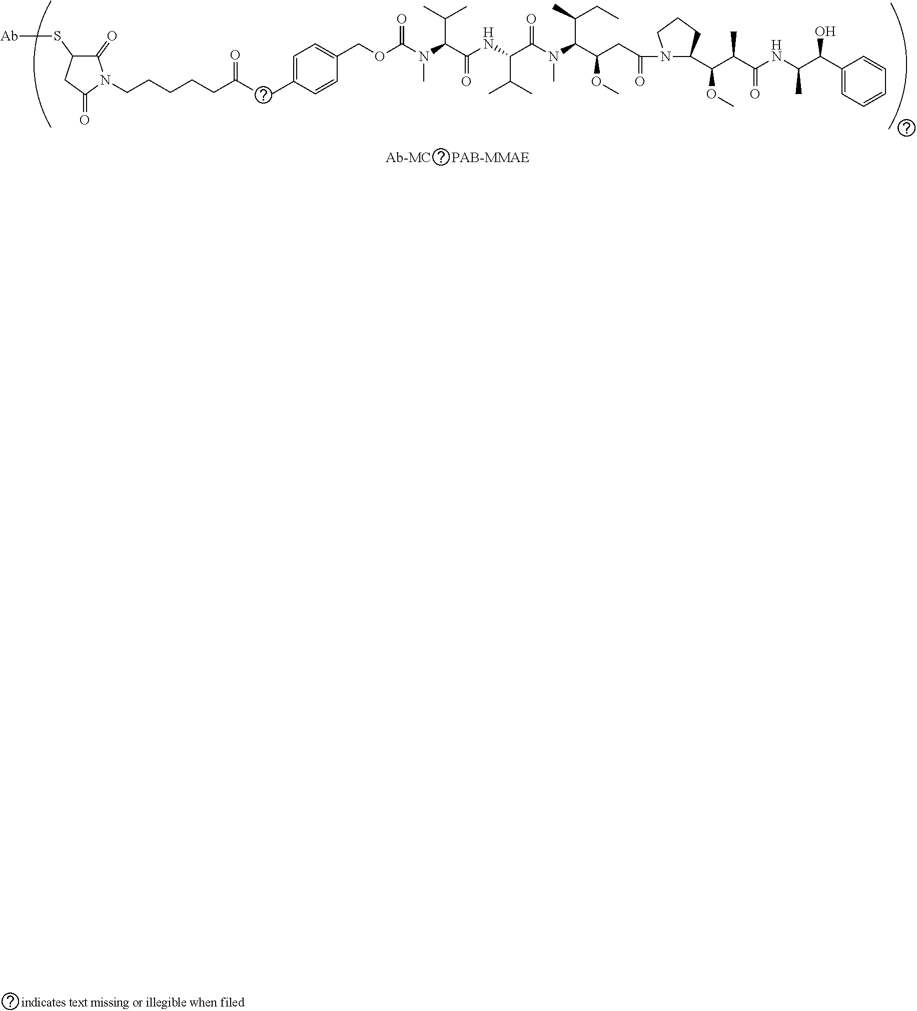

wherein p denotes a number from 1 to 8, S represents a sulphydryl residue of the anti-TF antibody, and Ab designates the anti-TF antibody or antigen-binding fragment thereof.
63B. The antibody-drug conjugate for use of embodiment 62B, wherein the average value of p in a population of the antibody-drug conjugates is about 4.
64B. The antibody-drug conjugate for use of any one of embodiments 1B-63B, wherein the antibody-drug conjugate is tisotumab vedotin.
65B. The antibody-drug conjugate for use of any one of embodiments 1B-64B, wherein the route of administration for the antibody-drug conjugate is intravenous.
66B. The antibody-drug conjugate for use of any one of embodiments 1B-65B, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
67B. The antibody-drug conjugate for use of any one of embodiments 1B-66B, wherein the anti-PD-1 antibody comprises a heavy chain variable region comprising the amino acid sequence of SEQ ID NO:31 and a light chain variable region comprising the amino acid sequence of SEQ ID NO:32.
68B. The antibody-drug conjugate for use of any one of embodiments 1B-67B, wherein the anti-PD-1 antibody comprises a heavy chain comprising the amino acid sequence of SEQ ID NO:33 and a light chain comprising the amino acid sequence of SEQ ID NO:34.
69B. The antibody-drug conjugate for use of any one of embodiments 1B-68B, wherein the anti-PD-1 antibody is pembrolizumab.
70B. The antibody-drug conjugate for use of any one of embodiments 1B-69B, wherein the route of administration for the anti-PD-1 antibody or antigen-binding fragment thereof is intravenous or subcutaneous.
71B. The antibody-drug conjugate for use any one of embodiments 1B-70B, wherein the route of administration for the anti-PD-1 antibody or antigen-binding fragment thereof is intravenous.
72B. The antibody-drug conjugate for use of any one of embodiments 1B-70B, wherein the route of administration for the anti-PD-1 antibody or antigen-binding fragment thereof is subcutaneous.
73B. The antibody-drug conjugate for use of any one of embodiments 1B-72B, wherein the anti-PD-1 antibody or antigen-binding fragment thereof and the antibody-drug conjugate are administered sequentially.
74B. The antibody-drug conjugate for use of any one of embodiments 1B-72B, wherein the anti-PD-1 antibody or antigen-binding fragment thereof and the antibody-drug conjugate are administered simultaneously.
75B. The antibody-drug conjugate for use of any one of embodiments 1B-74B, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
76B. The antibody-drug conjugate for use of any one of embodiments 1B-75B, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
77B. The antibody-drug conjugate for use of any one of embodiments 1B-76B, wherein the subject has a tumor that expresses PD-L1 (TPS≥1).
78B. The antibody-drug conjugate for use of any one of embodiments 1B-77B, wherein the subject has a tumor that has high PD-L1 expression (TPS≥50).
79B. The antibody-drug conjugate for use of any one of embodiments 1B-76B, wherein the subject has a tumor that expresses PD-L1 (CPS≥1).
80B. The antibody-drug conjugate for use of any one of embodiments 1B-79B, wherein a tumor derived from the cancer comprises one or more cells that express PD-L1, PD-L2, or both PD-L1 and PD-L2.
81B. The antibody-drug conjugate for use of any one of embodiments 1B-80B, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
82B. The antibody-drug conjugate for use of any one of embodiments 1B-81B, wherein one or more therapeutic effects in the subject is improved after administration of the antibody-drug conjugate and the anti-PD-1 antibody or antigen-binding fragment thereof relative to a baseline.
83B. The antibody-drug conjugate for use of embodiment 82B, wherein the one or more therapeutic effects is selected from the group consisting of: size of a tumor derived from the cancer, objective response rate, duration of response, time to response, progression free survival, and overall survival.
84B. The antibody-drug conjugate for use of any one of embodiments 1B-83B, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
85B. The antibody-drug conjugate for use of any one of embodiments 1B-84B, wherein the objective response rate is at least about 20%, at least about 25%, at least about 30%, at least about 35%, at least about 40%, at least about 45%, at least about 50%, at least about 60%, at least about 70%, or at least about 80%.
86B. The antibody-drug conjugate for use of any one of embodiments 1B-85B, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
87B. The antibody-drug conjugate for use of any one of embodiments 1B-86B, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
88B. The antibody-drug conjugate for use of any one of embodiments 1B-87B, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
89B. The antibody-drug conjugate for use of any one of embodiments 1B-88B, wherein the subject has one or more adverse events and is further administered an additional therapeutic agent to eliminate or reduce the severity of the one or more adverse events.
90B. The antibody-drug conjugate for use of any one of embodiments 1B-89B, wherein the subject is at risk of developing one or more adverse events and is further administered an additional therapeutic agent to prevent or reduce the severity of the one or more adverse events.
91B. The antibody-drug conjugate for use of any one of embodiments 89B-90B, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
92B. The antibody-drug conjugate for use of any one of embodiments 89B-91B, wherein the one or more adverse events is a grade 3 or greater adverse event.
93B. The antibody-drug conjugate for use of any one of embodiments 89B-91B, wherein the one or more adverse events is a serious adverse event.
94B. The antibody-drug conjugate for use of any one of embodiments 89B-91B, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
95B. The antibody-drug conjugate for use of any one of embodiments 1B-94B, wherein the subject is a human.
96B. The antibody-drug conjugate for use of any one of embodiments 1B-95B, wherein the antibody-drug conjugate is in a pharmaceutical composition comprising the antibody-drug conjugate and a pharmaceutical acceptable carrier.
97B. The antibody-drug conjugate for use of any one of embodiments 1B-96B, wherein the anti-PD-1 antibody or antigen-binding fragment thereof is in a pharmaceutical composition comprising the anti-PD-1 antibody or antigen-binding fragment thereof and a pharmaceutical acceptable carrier.

C. Use of an Antibody-Drug Conjugate

1C. Use of an antibody-drug conjugate that binds to TF for the manufacture of a medicament for treating cancer in a subject, wherein the medicament is for use in combination with an anti-PD-1 antibody or an antigen-binding fragment thereof, wherein the antibody-drug conjugate comprises an anti-TF antibody or an antigen-binding fragment thereof conjugated to monomethyl auristatin E, wherein the anti-PD-1 antibody or the antigen-binding fragment thereof inhibits PD-1 activity, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are defined by the Kabat numbering scheme;

and wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme, wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period.

2C. The use of embodiment 1C, wherein the antibody-drug conjugate is administered at a dose of about 0.65 mg/kg.
3C. The use of embodiment 1C, wherein the antibody-drug conjugate is administered at a dose of 0.65 mg/kg.
4C. The use of embodiment 1C, wherein the antibody-drug conjugate is administered at a dose of about 0.7 mg/kg.
5C. The use of embodiment 1C, wherein the antibody-drug conjugate is administered at a dose of 0.7 mg/kg.
6C. The use of embodiment 1C, wherein the antibody-drug conjugate is administered at a dose of about 0.8 mg/kg.
7C. The use of embodiment 1C, wherein the antibody-drug conjugate is administered at a dose of 0.8 mg/kg.
8C. The use of embodiment 1C, wherein the antibody-drug conjugate is administered at a dose of about 0.9 mg/kg.
9C. The use of embodiment 1C, wherein the antibody-drug conjugate is administered at a dose of 0.9 mg/kg.
10C. The use of embodiment 1C, wherein the antibody-drug conjugate is administered at a dose of about 1.0 mg/kg.
11C. The use of embodiment 1C, wherein the antibody-drug conjugate is administered at a dose of 1.0 mg/kg.
12C. The use of embodiment 1C, wherein the antibody-drug conjugate is administered at a dose of about 1.1 mg/kg.
13C. The use of embodiment 1C, wherein the antibody-drug conjugate is administered at a dose of 1.1 mg/kg.
14C. The use of embodiment 1C, wherein the antibody-drug conjugate is administered at a dose of about 1.2 mg/kg.
15C. The use of embodiment 1C, wherein the antibody-drug conjugate is administered at a dose of 1.2 mg/kg.
16C. The use of embodiment 1C, wherein the antibody-drug conjugate is administered at a dose of about 1.3 mg/kg.
17C. The use of embodiment 1C, wherein the antibody-drug conjugate is administered at a dose of 1.3 mg/kg.
18C. The use of embodiment 1C, wherein the antibody-drug conjugate is administered at a dose of about 1.4 mg/kg.
19C. The use of embodiment 1C, wherein the antibody-drug conjugate is administered at a dose of 1.4 mg/kg.
20C. The use of embodiment 1C, wherein the antibody-drug conjugate is administered at a dose of about 1.5 mg/kg.
21C. The use of embodiment 1C, wherein the antibody-drug conjugate is administered at a dose of 1.5 mg/kg.
22C. The use of any one of embodiments 1C-21C, wherein the antibody-drug conjugate is administered once every 1 week for 3 consecutive weeks followed by a 1 week rest period without any administration of the antibody-drug conjugate so that each cycle time is 28 days including the resting period.
23C. The use of any one of embodiments 1C-21C, wherein the antibody-drug conjugate is administered on about days 1, 8, and 15 of about a 4-week cycle.
24C. The use of any one of embodiments 1C-21C, wherein the antibody-drug conjugate is administered on days 1, 8, and 15 of a 4-week cycle.
25C. The use of any one of embodiments 1C-24C, wherein the anti-PD-1 antibody or antigen-binding fragment thereof is administered at a flat dose ranging from about 50 mg to about 500 mg.
26C. The use of any one of embodiments 1C-25C, wherein the anti-PD-1 antibody or antigen-binding fragment thereof is administered at a flat dose of about 200 mg.
27C. The use of any one of embodiments 1C-25C, wherein the anti-PD-1 antibody or antigen-binding fragment thereof is administered at a flat dose of 200 mg.
28C. The use of any one of embodiments 1C-25C, wherein the anti-PD-1 antibody or antigen-binding fragment thereof is administered at a flat dose of about 400 mg.
29C. The use of any one of embodiments 1C-25C, wherein the anti-PD-1 antibody or antigen-binding fragment thereof is administered at a flat dose of 400 mg.
30C. The use of any one of embodiments 1C-29C, wherein the anti-PD-1 antibody or antigen-binding fragment thereof is administered once about every 1 week, once about every 2 weeks, once about every 3 weeks, once about every 4 weeks, once about every 5 weeks, or once about every 6 weeks.
31C. The use of embodiment 30C, wherein the anti-PD-1 antibody or antigen-binding fragment thereof is administered once about every 3 weeks.
32C. The use of embodiment 30C, wherein the anti-PD-1 antibody or antigen-binding fragment thereof is administered once every 3 weeks.
33C. The use of embodiment 30C, wherein the anti-PD-1 antibody or antigen-binding fragment thereof is administered once about every 6 weeks.
34C. The use of embodiment 30C, wherein the anti-PD-1 antibody or antigen-binding fragment thereof is administered once every 6 weeks.
35C. The use of any one of embodiments 1C-30C, wherein the anti-PD-1 antibody or antigen-binding fragment thereof is administered on about day 1 of about a 21-day cycle.
36C. The use of any one of embodiments 1C-30C, wherein the anti-PD-1 antibody or antigen-binding fragment thereof is administered on day 1 of a 21-day cycle.
37C. The use of any one of embodiments 1C-30C, wherein the anti-PD-1 antibody or antigen-binding fragment thereof is administered on about day 1 of about a 6-week cycle.
38C. The use of any one of embodiments 1C-30C, wherein the anti-PD-1 antibody or antigen-binding fragment thereof is administered on day 1 of a 6-week cycle.
39C. The use of any one of embodiments 1C-38C, wherein the cancer is breast cancer.
40C. The use of embodiment 39C, wherein the breast cancer is ER+/HER2− breast cancer or triple negative breast cancer.
41C. The use of any one of embodiments 1C-38C, wherein the cancer is cervical cancer.
42C. The use of embodiment 41C, wherein the subject is not a candidate for curative therapy.
43C. The use of embodiment 42C, wherein curative therapy comprises radiotherapy and/or exenterative surgery.
44C. The use of embodiment 43C, wherein the subject has not received prior systemic therapy for the cervical cancer.
45C. The use of any one of embodiments 41C-44C, wherein the cervical cancer is a non-squamous cell carcinoma, an adenocarcinoma, an adenosquamous carcinoma or a squamous cell carcinoma.
46C. The use of embodiment 45C, wherein the cervical cancer is an adenocarcinoma.
47C. The use of embodiment 45C, wherein the cervical cancer is an adenosquamous carcinoma.
48C. The use of embodiment 45C, wherein the cervical cancer is a squamous cell carcinoma.
49C. The use of embodiment 45C, wherein the cervical cancer is a non-squamous cell carcinoma.
50C. The use of any one of embodiments 41C-49C, wherein the cervical cancer is an advanced stage cervical cancer.
51C. The use of embodiment 50C, wherein the advanced stage cervical cancer is a stage 3 or stage 4 cervical cancer.
52C. The use of embodiment 50C or 51C, wherein the advanced stage cervical cancer is metastatic cervical cancer.
53C. The use of any one of embodiments 41C-52C, wherein the cervical cancer is recurrent cervical cancer.
54C. The use of any one of embodiments 1C-53C, wherein the anti-TF antibody or antigen-binding fragment thereof of the antibody-drug conjugate is a monoclonal antibody or a monoclonal antigen-binding fragment thereof.
55C. The use of any one of embodiments 1C-54C, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
56C. The use of any one of embodiments 1C-55C, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
57C. The use of any one of embodiments 1C-56C, wherein the anti-TF antibody of the antibody-drug conjugate is tisotumab.
58C. The use of any one of embodiments 1C-57C, wherein the antibody-drug conjugate further comprises a linker between the anti-TF antibody or antigen-binding fragment thereof and the monomethyl auristatin E.
59C. The use of embodiment 58C, wherein the linker is a cleavable peptide linker.
60C. The use of embodiment 59C, wherein the cleavable peptide linker has a formula: -MC-vc-PAB-, wherein:

a) MC is:

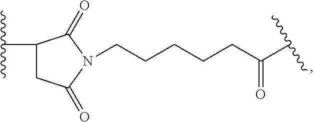 [SMILES: *C(=O)CCCCCN1C(=O)CC(*)C1=O]

b) vc is the dipeptide valine-citrulline, and

c) PAB is:

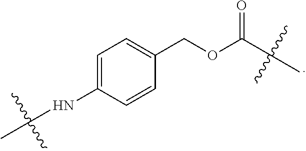 [SMILES: *Nc1ccc(COC(*)=O)cc1]

61C. The use of any one of embodiments 58C-60C, wherein the linker is attached to sulphydryl residues of the anti-TF antibody or antigen-binding fragment thereof obtained by partial reduction or full reduction of the anti-TF antibody or antigen-binding fragment thereof.
62C. The use of embodiment 61C, wherein the linker is attached to MMAE, wherein the antibody-drug conjugate has the following structure:

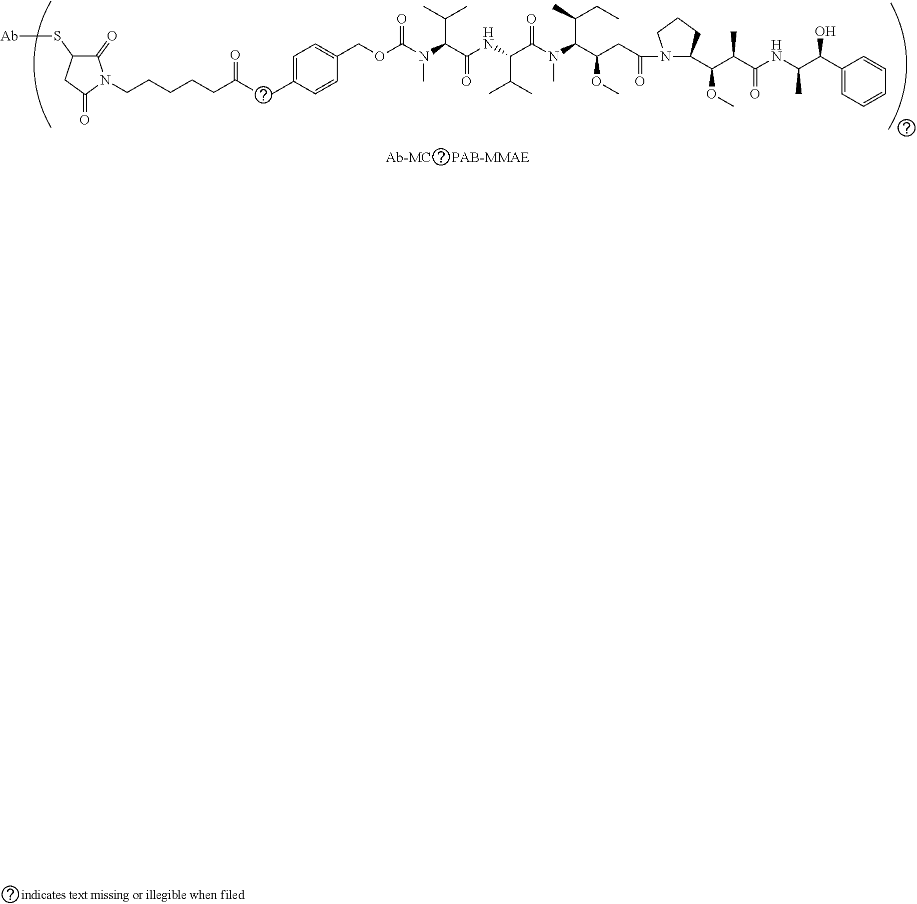

wherein p denotes a number from 1 to 8, S represents a sulphydryl residue of the anti-TF antibody, and Ab designates the anti-TF antibody or antigen-binding fragment thereof.
63C. The use of embodiment 62C, wherein the average value of p in a population of the antibody-drug conjugates is about 4.
64C. The use of any one of embodiments 1C-63C, wherein the antibody-drug conjugate is tisotumab vedotin.
65C. The use of any one of embodiments 1C-64C, wherein the route of administration for the antibody-drug conjugate is intravenous.
66C. The use of any one of embodiments 1C-65C, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
67C. The use of any one of embodiments 1C-66C, wherein the anti-PD-1 antibody comprises a heavy chain variable region comprising the amino acid sequence of SEQ ID NO:31 and a light chain variable region comprising the amino acid sequence of SEQ ID NO:32.
68C. The use of any one of embodiments 1C-67C, wherein the anti-PD-1 antibody comprises a heavy chain comprising the amino acid sequence of SEQ ID NO:33 and a light chain comprising the amino acid sequence of SEQ ID NO:34.
69C. The use of any one of embodiments 1C-68C, wherein the anti-PD-1 antibody is pembrolizumab.
70C. The use of any one of embodiments 1C-69C, wherein the route of administration for the anti-PD-1 antibody or antigen-binding fragment thereof is intravenous or subcutaneous.
71C. The use any one of embodiments 1C-69C, wherein the route of administration for the anti-PD-1 antibody or antigen-binding fragment thereof is intravenous.
72C. The use of any one of embodiments 1C-69C, wherein the route of administration for the anti-PD-1 antibody or antigen-binding fragment thereof is subcutaneous.
73C. The use of any one of embodiments 1C-72C, wherein the anti-PD-1 antibody or antigen-binding fragment thereof and the antibody-drug conjugate are administered sequentially.
74C. The use of any one of embodiments 1C-72C, wherein the anti-PD-1 antibody or antigen-binding fragment thereof and the antibody-drug conjugate are administered simultaneously.
75C. The use of any one of embodiments 1C-74C, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
76C. The use of any one of embodiments 1C-75C, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
77C. The use of any one of embodiments 1C-76C, wherein the subject has a tumor that expresses PD-L1 (TPS≥1).
78C. The use of any one of embodiments 1C-77C, wherein the subject has a tumor that has high PD-L1 expression (TPS≥50).
79C. The use of any one of embodiments 1C-76C, wherein the subject has a tumor that expresses PD-L1 (CPS≥1).
809C. The use of any one of embodiments 1C-79C, wherein a tumor derived from the cancer comprises one or more cells that express PD-L1, PD-L2, or both PD-L1 and PD-L2.
81C. The use of any one of embodiments 1C-80C, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
82C. The use of any one of embodiments 1C-81C, wherein one or more therapeutic effects in the subject is improved after administration of the antibody-drug conjugate and the anti-PD-1 antibody or antigen-binding fragment thereof relative to a baseline.
83C. The use of embodiment 82C, wherein the one or more therapeutic effects is selected from the group consisting of: size of a tumor derived from the cancer, objective response rate, duration of response, time to response, progression free survival, and overall survival.
84C. The use of any one of embodiments 1C-83C, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
85C. The use of any one of embodiments 1C-84C, wherein the objective response rate is at least about 20%, at least about 25%, at least about 30%, at least about 35%, at least about 40%, at least about 45%, at least about 50%, at least about 60%, at least about 70%, or at least about 80%.
86C. The use of any one of embodiments 1C-85C, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
87C. The use of any one of embodiments 1C-86C, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
88C. The use of any one of embodiments 1C-87C, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
89C. The use of any one of embodiments 1C-88C, wherein the subject has one or more adverse events and is further administered an additional therapeutic agent to eliminate or reduce the severity of the one or more adverse events.
90C. The use of any one of embodiments 1C-89C, wherein the subject is at risk of developing one or more adverse events and is further administered an additional therapeutic agent to prevent or reduce the severity of the one or more adverse events.
91C. The use of any one of embodiments 89C-90C, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
92C. The use of any one of embodiments 89C-91C, wherein the one or more adverse events is a grade 3 or greater adverse event.
93C. The use of any one of embodiments 89C-91C, wherein the one or more adverse events is a serious adverse event.
94C. The use of any one of embodiments 89C-90C, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
95C. The use of any one of embodiments 1C-94C, wherein the subject is a human.
96C. The use of any one of embodiments 1C-95C, wherein the antibody-drug conjugate is in a pharmaceutical composition comprising the antibody-drug conjugate and a pharmaceutical acceptable carrier.
97C. The use of any one of embodiments 1C-96C, wherein the anti-PD-1 antibody or antigen-binding fragment thereof is in a pharmaceutical composition comprising the anti-PD-1 antibody or antigen-binding fragment thereof and a pharmaceutical acceptable carrier.

D. Anti-PD-1 Antibody or an Antigen-Binding Fragment Thereof for Use

1D. An anti-PD-1 antibody or an antigen-binding fragment thereof for use in the treatment of cancer in a subject, wherein the anti-PD-1 antibody is for administration, or to be administered in combination with an antibody-drug conjugate that binds to TF wherein the antibody-drug conjugate comprises an anti-TF antibody or an antigen-binding fragment thereof conjugated to monomethyl auristatin E, wherein the anti-PD-1 antibody or the antigen-binding fragment thereof inhibits PD-1 activity, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are defined by the Kabat numbering scheme;

and wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof are defined by the IMGT numbering scheme, wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period.

2D. The anti-PD-1 antibody or an antigen-binding fragment thereof for use of embodiment 1D, wherein the antibody-drug conjugate is administered at a dose of about 0.65 mg/kg.
3D. The anti-PD-1 antibody or an antigen-binding fragment thereof for use of embodiment 1D, wherein the antibody-drug conjugate is administered at a dose of 0.65 mg/kg.
4D. The anti-PD-1 antibody or an antigen-binding fragment thereof for use embodiment 1D, wherein the antibody-drug conjugate is administered at a dose of about 0.7 mg/kg.
5D. The anti-PD-1 antibody or an antigen-binding fragment thereof for use of embodiment 1D, wherein the antibody-drug conjugate is administered at a dose of 0.7 mg/kg.
6D. The anti-PD-1 antibody or an antigen-binding fragment thereof for use of embodiment 1D, wherein the antibody-drug conjugate is administered at a dose of about 0.8 mg/kg.
7D. The anti-PD-1 antibody or an antigen-binding fragment thereof for use of embodiment 1D, wherein the antibody-drug conjugate is administered at a dose of 0.8 mg/kg.
8D. The anti-PD-1 antibody or an antigen-binding fragment thereof for use of embodiment 1D, wherein the antibody-drug conjugate is administered at a dose of about 0.9 mg/kg.
9D. The anti-PD-1 antibody or an antigen-binding fragment thereof for use of embodiment 1D, wherein the antibody-drug conjugate is administered at a dose of 0.9 mg/kg.
10D. The anti-PD-1 antibody or an antigen-binding fragment thereof for use of embodiment 1D, wherein the antibody-drug conjugate is administered at a dose of about 1.0 mg/kg.
11D. The anti-PD-1 antibody or an antigen-binding fragment thereof for use of embodiment 1D, wherein the antibody-drug conjugate is administered at a dose of 1.0 mg/kg.
12D. The anti-PD-1 antibody or an antigen-binding fragment thereof for use of embodiment 1D, wherein the antibody-drug conjugate is administered at a dose of about 1.1 mg/kg.
13D. The anti-PD-1 antibody or an antigen-binding fragment thereof for use of embodiment 1D, wherein the antibody-drug conjugate is administered at a dose of 1.1 mg/kg.
14D. The anti-PD-1 antibody or an antigen-binding fragment thereof for use of embodiment 1D, wherein the antibody-drug conjugate is administered at a dose of about 1.2 mg/kg.
15D. The anti-PD-1 antibody or an antigen-binding fragment thereof for use of embodiment 1D, wherein the antibody-drug conjugate is administered at a dose of 1.2 mg/kg.
16D. The anti-PD-1 antibody or an antigen-binding fragment thereof for use of embodiment 1D, wherein the antibody-drug conjugate is administered at a dose of about 1.3 mg/kg.
17D. The anti-PD-1 antibody or an antigen-binding fragment thereof for use of embodiment 1D, wherein the antibody-drug conjugate is administered at a dose of 1.3 mg/kg.
18D. The anti-PD-1 antibody or an antigen-binding fragment thereof for use of embodiment 1D, wherein the antibody-drug conjugate is administered at a dose of about 1.4 mg/kg.
19D. The anti-PD-1 antibody or an antigen-binding fragment thereof for use of embodiment 1D, wherein the antibody-drug conjugate is administered at a dose of 1.4 mg/kg.
20D. The anti-PD-1 antibody or an antigen-binding fragment thereof for use of embodiment 1D, wherein the antibody-drug conjugate is administered at a dose of about 1.5 mg/kg.
21D. The anti-PD-1 antibody or an antigen-binding fragment thereof for use of embodiment 1D, wherein the antibody-drug conjugate is administered at a dose of 1.5 mg/kg.
22D. The anti-PD-1 antibody or an antigen-binding fragment thereof for use of any one of embodiments 1D-21D, wherein the antibody-drug conjugate is administered once every 1 week for 3 consecutive weeks followed by a 1 week rest period without any administration of the antibody-drug conjugate so that each cycle time is 28 days including the resting period.
23D. The anti-PD-1 antibody or an antigen-binding fragment thereof for use of any one of embodiments 1D-21D, wherein the antibody-drug conjugate is administered on about days 1, 8, and 15 of about a 4-week cycle.
24D. The anti-PD-1 antibody or an antigen-binding fragment thereof for use of any one of embodiments 1D-21D, wherein the antibody-drug conjugate is administered on days 1, 8, and 15 of a 4-week cycle.
25D. The anti-PD-1 antibody or an antigen-binding fragment thereof for use of any one of embodiments 1D-24D, wherein the anti-PD-1 antibody or antigen-binding fragment thereof is administered at a flat dose ranging from about 50 mg to about 500 mg.
26D. The anti-PD-1 antibody or an antigen-binding fragment thereof for use of any one of embodiments 1D-25D, wherein the anti-PD-1 antibody or antigen-binding fragment thereof is administered at a flat dose of about 200 mg.
27D. The anti-PD-1 antibody or an antigen-binding fragment thereof for use of any one of embodiments 1D-25D, wherein the anti-PD-1 antibody or antigen-binding fragment thereof is administered at a flat dose of 200 mg.
28D. The anti-PD-1 antibody or an antigen-binding fragment thereof for use of any one of embodiments 1D-25D, wherein the anti-PD-1 antibody or antigen-binding fragment thereof is administered at a flat dose of about 400 mg.
29D. The anti-PD-1 antibody or an antigen-binding fragment thereof for use of any one of embodiments 1D-25D, wherein the anti-PD-1 antibody or antigen-binding fragment thereof is administered at a flat dose of 400 mg.
30D. The anti-PD-1 antibody or an antigen-binding fragment thereof for use of any one of embodiments 1D-29D, wherein the anti-PD-1 antibody or antigen-binding fragment thereof is administered once about every 1 week, once about every 2 weeks, once about every 3 weeks, once about every 4 weeks, once about every 5 weeks, or once about every 6 weeks.
31D. The anti-PD-1 antibody or an antigen-binding fragment thereof for use of embodiment 30D, wherein the anti-PD-1 antibody or antigen-binding fragment thereof is administered once about every 3 weeks.
32D. The anti-PD-1 antibody or an antigen-binding fragment thereof for use of embodiment 30D, wherein the anti-PD-1 antibody or antigen-binding fragment thereof is administered once every 3 weeks.
33D. The anti-PD-1 antibody or an antigen-binding fragment thereof for use of embodiment 30D, wherein the anti-PD-1 antibody or antigen-binding fragment thereof is administered once about every 6 weeks.
34D. The anti-PD-1 antibody or an antigen-binding fragment thereof for use of embodiment 30D, wherein the anti-PD-1 antibody or antigen-binding fragment thereof is administered once every 6 weeks.
35D. The anti-PD-1 antibody or an antigen-binding fragment thereof for use of any one of embodiments 1D-30D, wherein the anti-PD-1 antibody or antigen-binding fragment thereof is administered on about day 1 of about a 21-day cycle.
36D. The anti-PD-1 antibody or an antigen-binding fragment thereof for use of any one of embodiments 1D-30D, wherein the anti-PD-1 antibody or antigen-binding fragment thereof is administered on day 1 of a 21-day cycle.
37D. The anti-PD-1 antibody or an antigen-binding fragment thereof for use of any one of embodiments 1D-30D, wherein the anti-PD-1 antibody or antigen-binding fragment thereof is administered on about day 1 of about a 6-week cycle.
38D. The anti-PD-1 antibody or an antigen-binding fragment thereof for use of any one of embodiments 1D-30D, wherein the anti-PD-1 antibody or antigen-binding fragment thereof is administered on day 1 of a 6-week cycle.
39D. The anti-PD-1 antibody or an antigen-binding fragment thereof for use of any one of embodiments 1D-38D, wherein the cancer is breast cancer.
40D. The anti-PD-1 antibody or an antigen-binding fragment thereof for use of embodiment 39D, wherein the breast cancer is ER+/HER2− breast cancer or triple negative breast cancer.
41D. The anti-PD-1 antibody or an antigen-binding fragment thereof for use of any one of embodiments 1D-38D, wherein the cancer is cervical cancer.
42D. The anti-PD-1 antibody or an antigen-binding fragment thereof for use of embodiment 41D, wherein the subject is not a candidate for curative therapy.
43D. The anti-PD-1 antibody or an antigen-binding fragment thereof for use of embodiment 42D, wherein curative therapy comprises radiotherapy and/or exenterative surgery.
44D. The anti-PD-1 antibody or an antigen-binding fragment thereof for use of embodiment 43D, wherein the subject has not received prior systemic therapy for the cervical cancer.
45D. The anti-PD-1 antibody or an antigen-binding fragment thereof for use of any one of embodiments 41D-44D, wherein the cervical cancer is a non-squamous cell carcinoma, an adenocarcinoma, an adenosquamous carcinoma or a squamous cell carcinoma.
46D. The anti-PD-1 antibody or an antigen-binding fragment thereof for use of embodiment 45D, wherein the cervical cancer is an adenocarcinoma.
47D. The anti-PD-1 antibody or an antigen-binding fragment thereof for use of embodiment 45D, wherein the cervical cancer is an adenosquamous carcinoma.
48D. The anti-PD-1 antibody or an antigen-binding fragment thereof for use of embodiment 45D, wherein the cervical cancer is a squamous cell carcinoma.
49D. The anti-PD-1 antibody or an antigen-binding fragment thereof for use of embodiment 45D, wherein the cervical cancer is a non-squamous cell carcinoma.
50D. The anti-PD-1 antibody or an antigen-binding fragment thereof for use of any one of embodiments 41D-49D, wherein the cervical cancer is an advanced stage cervical cancer.
51D. The anti-PD-1 antibody or an antigen-binding fragment thereof for use of embodiment 50D, wherein the advanced stage cervical cancer is a stage 3 or stage 4 cervical cancer.
52D. The anti-PD-1 antibody or an antigen-binding fragment thereof for use of embodiment 50D or 51D, wherein the advanced stage cervical cancer is metastatic cervical cancer.
53D. The anti-PD-1 antibody or an antigen-binding fragment thereof for use of any one of embodiments 41D-52D, wherein the cervical cancer is recurrent cervical cancer.
54D. The anti-PD-1 antibody or an antigen-binding fragment thereof for use of any one of embodiments 1D-53D, wherein the anti-TF antibody or antigen-binding fragment thereof of the antibody-drug conjugate is a monoclonal antibody or a monoclonal antigen-binding fragment thereof.
55D. The anti-PD-1 antibody or an antigen-binding fragment thereof for use of any one of embodiments 1D-54D, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
56D. The anti-PD-1 antibody or an antigen-binding fragment thereof for use of any one of embodiments 1D-55D, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
57D. The anti-PD-1 antibody or an antigen-binding fragment thereof for use of any one of embodiments 1D-56D, wherein the anti-TF antibody of the antibody-drug conjugate is tisotumab.
58D. The anti-PD-1 antibody or an antigen-binding fragment thereof for use of any one of embodiments 1D-57D, wherein the antibody-drug conjugate further comprises a linker between the anti-TF antibody or antigen-binding fragment thereof and the monomethyl auristatin E.
59D. The anti-PD-1 antibody or an antigen-binding fragment thereof for use of embodiment 58D, wherein the linker is a cleavable peptide linker.
60D. The anti-PD-1 antibody or an antigen-binding fragment thereof for use of embodiment 59D, wherein the cleavable peptide linker has a formula: -MC-vc-PAB-, wherein:

a) MC is:

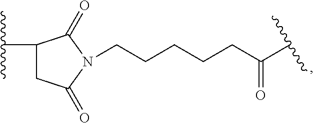 [SMILES: *C(=O)CCCCCN1C(=O)CC(*)C1=O]

b) vc is the dipeptide valine-citrulline, and

c) PAB is:

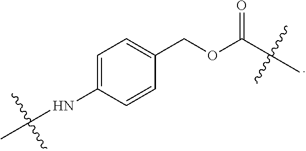 [SMILES: *Nc1ccc(COC(*)=O)cc1]

61D. The anti-PD-1 antibody or an antigen-binding fragment thereof for use of any one of embodiments 58D-60D, wherein the linker is attached to sulphydryl residues of the anti-TF antibody or antigen-binding fragment thereof obtained by partial reduction or full reduction of the anti-TF antibody or antigen-binding fragment thereof.
62D. The anti-PD-1 antibody or an antigen-binding fragment thereof for use of embodiment 61D, wherein the linker is attached to MMAE, wherein the antibody-drug conjugate has the following structure:

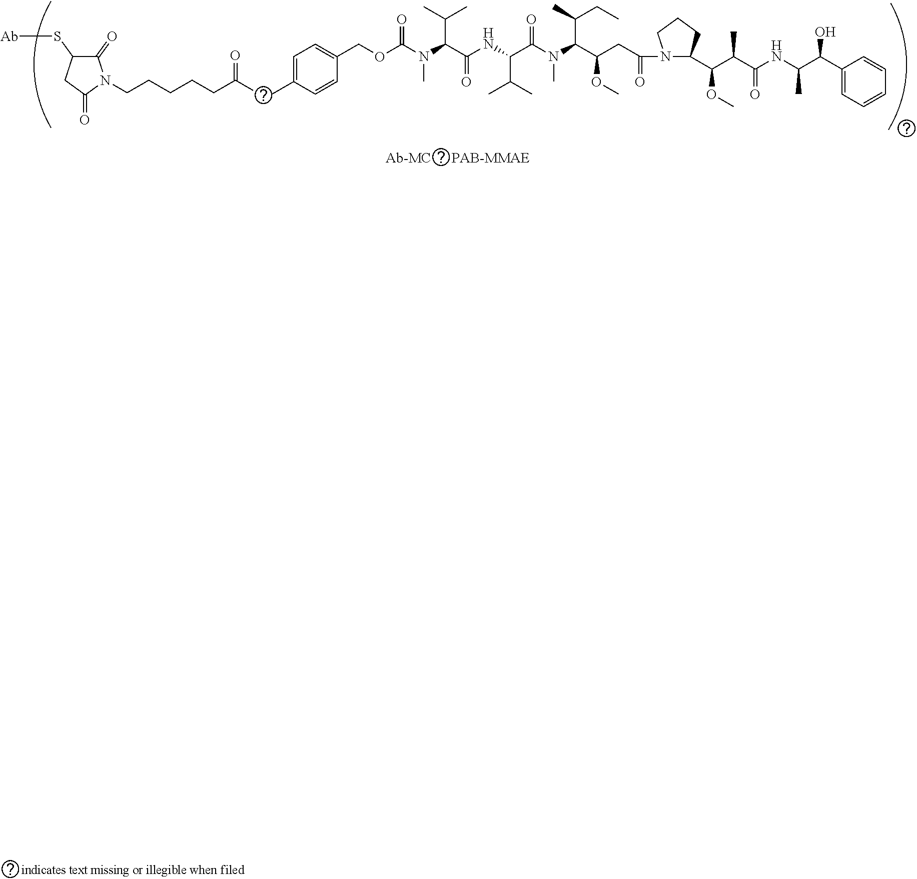

wherein p denotes a number from 1 to 8, S represents a sulphydryl residue of the anti-TF antibody, and Ab designates the anti-TF antibody or antigen-binding fragment thereof.
63D. The anti-PD-1 antibody or an antigen-binding fragment thereof for use of embodiment 62D, wherein the average value of p in a population of the antibody-drug conjugates is about 4.
64D. The anti-PD-1 antibody or an antigen-binding fragment thereof for use of any one of embodiments 1D-63D, wherein the antibody-drug conjugate is tisotumab vedotin.
65D. The anti-PD-1 antibody or an antigen-binding fragment thereof for use of any one of embodiments 1D-64D, wherein the route of administration for the antibody-drug conjugate is intravenous.
66D. The anti-PD-1 antibody or an antigen-binding fragment thereof for use of any one of embodiments 1D-65D, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
67D. The anti-PD-1 antibody or an antigen-binding fragment thereof for use of any one of embodiments 1D-66D, wherein the anti-PD-1 antibody comprises a heavy chain variable region comprising the amino acid sequence of SEQ ID NO:31 and a light chain variable region comprising the amino acid sequence of SEQ ID NO:32.
68D. The anti-PD-1 antibody or an antigen-binding fragment thereof for use of any one of embodiments 1D-67D, wherein the anti-PD-1 antibody comprises a heavy chain comprising the amino acid sequence of SEQ ID NO:33 and a light chain comprising the amino acid sequence of SEQ ID NO:34.
69D. The anti-PD-1 antibody or an antigen-binding fragment thereof for use of any one of embodiments 1D-68D, wherein the anti-PD-1 antibody is pembrolizumab.
70D. The anti-PD-1 antibody or an antigen-binding fragment thereof for use of any one of embodiments 1D-69D, wherein the route of administration for the anti-PD-1 antibody or antigen-binding fragment thereof is intravenous or subcutaneous.
71D. The anti-PD-1 antibody or an antigen-binding fragment thereof for use any one of embodiments 1D-69D, wherein the route of administration for the anti-PD-1 antibody or antigen-binding fragment thereof is intravenous.
72D. The anti-PD-1 antibody or an antigen-binding fragment thereof for use of any one of embodiments 1D-69D, wherein the route of administration for the anti-PD-1 antibody or antigen-binding fragment thereof is subcutaneous.
73D. The anti-PD-1 antibody or an antigen-binding fragment thereof for use of any one of embodiments 1D-72D, wherein the anti-PD-1 antibody or antigen-binding fragment thereof and the antibody-drug conjugate are administered sequentially.
74D. The anti-PD-1 antibody or an antigen-binding fragment thereof for use of any one of embodiments 1D-72D, wherein the anti-PD-1 antibody or antigen-binding fragment thereof and the antibody-drug conjugate are administered simultaneously.
75D. The anti-PD-1 antibody or an antigen-binding fragment thereof for use of any one of embodiments 1D-74D, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
76D. The anti-PD-1 antibody or an antigen-binding fragment thereof for use of any one of embodiments 1D-75D, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
77D. The anti-PD-1 antibody or an antigen-binding fragment thereof for use of any one of embodiments 1D-76D, wherein the subject has a tumor that expresses PD-L1 (TPS≥1).
78D. The anti-PD-1 antibody or an antigen-binding fragment thereof for use of any one of embodiments 1D-77D, wherein the subject has a tumor that has high PD-L1 expression (TPS≥50).
79D. The anti-PD-1 antibody or an antigen-binding fragment thereof for use of any one of embodiments 1D-76D, wherein the subject has a tumor that expresses PD-L1 (CPS≥1).
80D. The anti-PD-1 antibody or an antigen-binding fragment thereof for use of any one of embodiments 1D-79D, wherein a tumor derived from the cancer comprises one or more cells that express PD-L1, PD-L2, or both PD-L1 and PD-L2.
81D. The anti-PD-1 antibody or an antigen-binding fragment thereof for use of any one of embodiments 1D-80D, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
82D. The anti-PD-1 antibody or an antigen-binding fragment thereof for use of any one of embodiments 1D-81D, wherein one or more therapeutic effects in the subject is improved after administration of the antibody-drug conjugate and the anti-PD-1 antibody or antigen-binding fragment thereof relative to a baseline.
83D. The anti-PD-1 antibody or an antigen-binding fragment thereof for use of embodiment 82D, wherein the one or more therapeutic effects is selected from the group consisting of: size of a tumor derived from the cancer, objective response rate, duration of response, time to response, progression free survival, and overall survival.
84D. The anti-PD-1 antibody or an antigen-binding fragment thereof for use of any one of embodiments 1D-83D, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
85D. The anti-PD-1 antibody or an antigen-binding fragment thereof for use of any one of embodiments 1D-84D, wherein the objective response rate is at least about 20%, at least about 25%, at least about 30%, at least about 35%, at least about 40%, at least about 45%, at least about 50%, at least about 60%, at least about 70%, or at least about 80%.
86D. The anti-PD-1 antibody or an antigen-binding fragment thereof for use of any one of embodiments 1D-85D, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
87D. The anti-PD-1 antibody or an antigen-binding fragment thereof for use of any one of embodiments 1D-86D, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
88D. The anti-PD-1 antibody or an antigen-binding fragment thereof for use of any one of embodiments 1D-87D, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
89D. The anti-PD-1 antibody or an antigen-binding fragment thereof for use of any one of embodiments 1D-88D, wherein the subject has one or more adverse events and is further administered an additional therapeutic agent to eliminate or reduce the severity of the one or more adverse events.
90D. The anti-PD-1 antibody or an antigen-binding fragment thereof for use of any one of embodiments 1D-89D, wherein the subject is at risk of developing one or more adverse events and is further administered an additional therapeutic agent to prevent or reduce the severity of the one or more adverse events.
91D. The anti-PD-1 antibody or an antigen-binding fragment thereof for use of any one of embodiments 89D-90D, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
92D. The anti-PD-1 antibody or an antigen-binding fragment thereof for use of any one of embodiments 89D-91D, wherein the one or more adverse events is a grade 3 or greater adverse event.
93D. The anti-PD-1 antibody or an antigen-binding fragment thereof for use of any one of embodiments 89D-91D, wherein the one or more adverse events is a serious adverse event.
94D. The anti-PD-1 antibody or an antigen-binding fragment thereof for use of any one of embodiments 89D-91D, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
95D. The anti-PD-1 antibody or an antigen-binding fragment thereof for use of any one of embodiments 1D-94D, wherein the subject is a human.
96D. The anti-PD-1 antibody or an antigen-binding fragment thereof for use of any one of embodiments 1D-95D, wherein the antibody-drug conjugate is in a pharmaceutical composition comprising the antibody-drug conjugate and a pharmaceutical acceptable carrier.
97D. The anti-PD-1 antibody or an antigen-binding fragment thereof for use of any one of embodiments 1D-96D, wherein the anti-PD-1 antibody or antigen-binding fragment thereof is in a pharmaceutical composition comprising the anti-PD-1 antibody or antigen-binding fragment thereof and a pharmaceutical acceptable carrier.

E. Use of an Anti-PD-1 Antibody or an Antigen-Binding Fragment Thereof

1E. Use of an anti-PD-1 antibody or an antigen-binding fragment thereof for the manufacture of a medicament for treating cancer in a subject, wherein the medicament is for use in combination with an antibody-drug conjugate that binds to TF, wherein the antibody-drug conjugate comprises an anti-TF antibody or an antigen-binding fragment thereof conjugated to monomethyl auristatin E, wherein the anti-PD-1 antibody or the antigen-binding fragment thereof inhibits PD-1 activity, wherein the anti-PD-1 antibody or antigen-binding fragment thereof comprises a heavy chain variable region and a light chain variable region, wherein the heavy chain variable region comprises:

(i) a CDR-H1 comprising the amino acid sequence of SEQ ID NO: 17;

(ii) a CDR-H2 comprising the amino acid sequence of SEQ ID NO: 18; and

(iii) a CDR-H3 comprising the amino acid sequence of SEQ ID NO: 19; and

wherein the light chain variable region comprises:

(i) a CDR-L1 comprising the amino acid sequence of SEQ ID NO:20;

(ii) a CDR-L2 comprising the amino acid sequence of SEQ ID NO:21; and

(iii) a CDR-L3 comprising the amino acid sequence of SEQ ID NO:22, wherein the CDRs of the anti-PD-1 antibody or antigen-binding fragment thereof are defined by the Kabat numbering scheme;

and wherein the anti-TF antibody or antigen-binding fragment thereof comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO: 3; and

wherein the light chain variable region comprises:

(i) a CDR-L1 comprising the amino acid sequence of SEQ ID NO:4;

(ii) a CDR-L2 comprising the amino acid sequence of SEQ ID NO: 5; and

(iii) a CDR-L3 comprising the amino acid sequence of SEQ ID NO:6, wherein the CDRs of the anti-TF antibody or antigen-binding fragment thereof are defined by the IMGT numbering scheme, wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period.

2E. The use of embodiment 1E, wherein the antibody-drug conjugate is administered at a dose of about 0.65 mg/kg.
3E. The use of embodiment 1E, wherein the antibody-drug conjugate is administered at a dose of 0.65 mg/kg.
4E. The use of embodiment 1E, wherein the antibody-drug conjugate is administered at a dose of about 0.7 mg/kg.
5E. The use of embodiment 1E, wherein the antibody-drug conjugate is administered at a dose of 0.7 mg/kg.
6E. The use of embodiment 1E, wherein the antibody-drug conjugate is administered at a dose of about 0.8 mg/kg.
7E. The use of embodiment 1E, wherein the antibody-drug conjugate is administered at a dose of 0.8 mg/kg.
8E. The use of embodiment 1E, wherein the antibody-drug conjugate is administered at a dose of about 0.9 mg/kg.
9E. The use of embodiment 1E, wherein the antibody-drug conjugate is administered at a dose of 0.9 mg/kg.
10E. The use of embodiment 1E, wherein the antibody-drug conjugate is administered at a dose of about 1.0 mg/kg.
11E. The use of embodiment 1E, wherein the antibody-drug conjugate is administered at a dose of 1.0 mg/kg.
12E. The use of embodiment 1E, wherein the antibody-drug conjugate is administered at a dose of about 1.1 mg/kg.
13E. The use of embodiment 1E, wherein the antibody-drug conjugate is administered at a dose of 1.1 mg/kg.
14E. The use of embodiment 1E, wherein the antibody-drug conjugate is administered at a dose of about 1.2 mg/kg.
15E. The use of embodiment 1E, wherein the antibody-drug conjugate is administered at a dose of 1.2 mg/kg.
16E. The use of embodiment 1E, wherein the antibody-drug conjugate is administered at a dose of about 1.3 mg/kg.
17E. The use of embodiment 1E, wherein the antibody-drug conjugate is administered at a dose of 1.3 mg/kg.
18E. The use of embodiment 1E, wherein the antibody-drug conjugate is administered at a dose of about 1.4 mg/kg.
19E. The use of embodiment 1E, wherein the antibody-drug conjugate is administered at a dose of 1.4 mg/kg.
20E. The use of embodiment 1E, wherein the antibody-drug conjugate is administered at a dose of about 1.5 mg/kg.
21E. The use embodiment 1E, wherein the antibody-drug conjugate is administered at a dose of 1.5 mg/kg.
22E. The use of any one of embodiments 1E-21E, wherein the antibody-drug conjugate is administered once every 1 week for 3 consecutive weeks followed by a 1 week rest period without any administration of the antibody-drug conjugate so that each cycle time is 28 days including the resting period.
23E. The use of any one of embodiments 1E-21E, wherein the antibody-drug conjugate is administered on about days 1, 8, and 15 of about a 4-week cycle.
24E. The use of any one of embodiments 1E-21E, wherein the antibody-drug conjugate is administered on days 1, 8, and 15 of a 4-week cycle.
25E. The use of any one of embodiments 1E-24E, wherein the anti-PD-1 antibody or antigen-binding fragment thereof is administered at a flat dose ranging from about 50 mg to about 500 mg.
26E. The use of any one of embodiments 1E-25E, wherein the anti-PD-1 antibody or antigen-binding fragment thereof is administered at a flat dose of about 200 mg.
27E. The use of any one of embodiments 1E-25E, wherein the anti-PD-1 antibody or antigen-binding fragment thereof is administered at a flat dose of 200 mg.
28E. The use of any one of embodiments 1E-25E, wherein the anti-PD-1 antibody or antigen-binding fragment thereof is administered at a flat dose of about 400 mg.
29E. The use of any one of embodiments 1E-25E, wherein the anti-PD-1 antibody or antigen-binding fragment thereof is administered at a flat dose of 400 mg.
30E. The use of any one of embodiments 1E-29E, wherein the anti-PD-1 antibody or antigen-binding fragment thereof is administered once about every 1 week, once about every 2 weeks, once about every 3 weeks, once about every 4 weeks, once about every 5 weeks, or once about every 6 weeks.
31E. The use of embodiment 30E, wherein the anti-PD-1 antibody or antigen-binding fragment thereof is administered once about every 3 weeks.
32E. The use of embodiment 30E, wherein the anti-PD-1 antibody or antigen-binding fragment thereof is administered once every 3 weeks.
33E. The use of embodiment 30E, wherein the anti-PD-1 antibody or antigen-binding fragment thereof is administered once about every 6 weeks.
34E. The use of embodiment 30E, wherein the anti-PD-1 antibody or antigen-binding fragment thereof is administered once every 6 weeks.
35E. The use of any one of embodiments 1E-30E, wherein the anti-PD-1 antibody or antigen-binding fragment thereof is administered on about day 1 of about a 21-day cycle.
36E. The use of any one of embodiments 1E-30E, wherein the anti-PD-1 antibody or antigen-binding fragment thereof is administered on day 1 of a 21-day cycle.
37E. The use of any one of embodiments 1E-30E, wherein the anti-PD-1 antibody or antigen-binding fragment thereof is administered on about day 1 of about a 6-week cycle.
38E. The use of any one of embodiments 1E-30E, wherein the anti-PD-1 antibody or antigen-binding fragment thereof is administered on day 1 of a 6-week cycle.
39E. The use of any one of embodiments 1E-38E, wherein the cancer is breast cancer.
40E. The use of embodiment 39E, wherein the breast cancer is ER+/HER2− breast cancer or triple negative breast cancer.
41E. The use of any one of embodiments 1E-38E, wherein the cancer is cervical cancer.
42E. The use of embodiment 41E, wherein the subject is not a candidate for curative therapy.
43E. The use of embodiment 42E, wherein curative therapy comprises radiotherapy and/or exenterative surgery.
44E. The use of embodiment 43E, wherein the subject has not received prior systemic therapy for the cervical cancer.
45E. The use of any one of embodiments 41E-44E, wherein the cervical cancer is a non-squamous cell carcinoma an adenocarcinoma, an adenosquamous carcinoma or a squamous cell carcinoma.
46E. The use of embodiment 45E, wherein the cervical cancer is an adenocarcinoma.
47E. The use of embodiment 45E, wherein the cervical cancer is an adenosquamous carcinoma.
48E. The use of embodiment 45E, wherein the cervical cancer is a squamous cell carcinoma.
49E. The use of embodiment 45E, wherein the cervical cancer is a non-squamous cell carcinoma.
50E. The use of any one of embodiments 41E-49E, wherein the cervical cancer is an advanced stage cervical cancer.
51E. The use of embodiment 50E, wherein the advanced stage cervical cancer is a stage 3 or stage 4 cervical cancer.
52E. The use of embodiment 50E or 51E, wherein the advanced stage cervical cancer is metastatic cervical cancer.
53E. The use of any one of embodiments 41E-52E, wherein the cervical cancer is recurrent cervical cancer.
54E. The use of any one of embodiments 1E-53E, wherein the anti-TF antibody or antigen-binding fragment thereof of the antibody-drug conjugate is a monoclonal antibody or a monoclonal antigen-binding fragment thereof.
55E. The use of any one of embodiments 1E-54E, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
56E. The use of any one of embodiments 1E-55E, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
57E. The use of any one of embodiments 1E-56E, wherein the anti-TF antibody of the antibody-drug conjugate is tisotumab.
58E. The use of any one of embodiments 1E-57E, wherein the antibody-drug conjugate further comprises a linker between the anti-TF antibody or antigen-binding fragment thereof and the monomethyl auristatin E.
59E. The use of embodiment 58E, wherein the linker is a cleavable peptide linker.
60E. The use of embodiment 59E, wherein the cleavable peptide linker has a formula: -MC-vc-PAB-, wherein:

a) MC is:

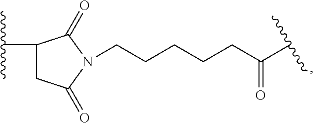 [SMILES: *C(=O)CCCCCN1C(=O)CC(*)C1=O]

b) vc is the dipeptide valine-citrulline, and

c) PAB is:

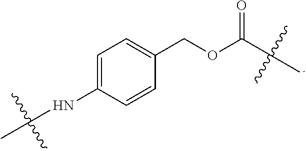 [SMILES: *Nc1ccc(COC(*)=O)cc1]

61E. The use of any one of embodiments 58E-60E, wherein the linker is attached to sulphydryl residues of the anti-TF antibody or antigen-binding fragment thereof obtained by partial reduction or full reduction of the anti-TF antibody or antigen-binding fragment thereof.
62E. The use of embodiment 61E, wherein the linker is attached to MMAE, wherein the antibody-drug conjugate has the following structure:

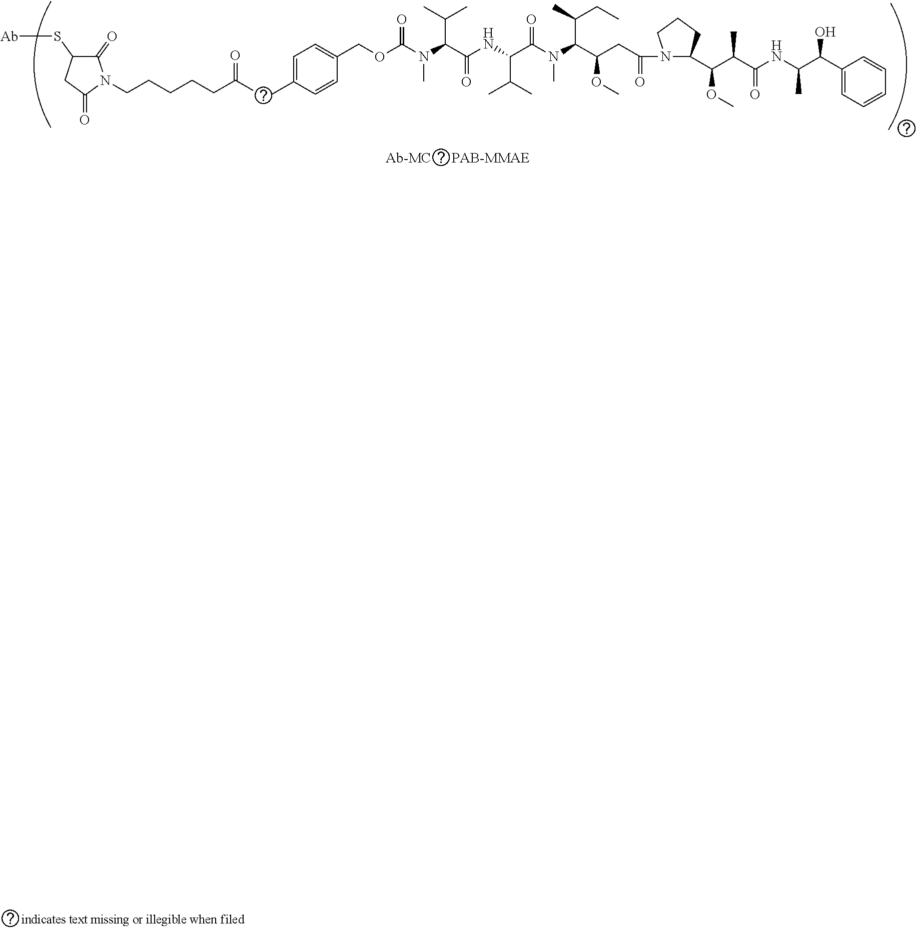

wherein p denotes a number from 1 to 8, S represents a sulphydryl residue of the anti-TF antibody, and Ab designates the anti-TF antibody or antigen-binding fragment thereof.
63E. The use of embodiment 62E, wherein the average value of p in a population of the antibody-drug conjugates is about 4.
64E. The use of any one of embodiments 1E-63E, wherein the antibody-drug conjugate is tisotumab vedotin.
65E. The use of any one of embodiments 1E-64E, wherein the route of administration for the antibody-drug conjugate is intravenous.
66E. The use of any one of embodiments 1E-65E, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
67E. The use of any one of embodiments 1E-66E, wherein the anti-PD-1 antibody comprises a heavy chain variable region comprising the amino acid sequence of SEQ ID NO:31 and a light chain variable region comprising the amino acid sequence of SEQ ID NO:32.
68E. The use of any one of embodiments 1E-67E, wherein the anti-PD-1 antibody comprises a heavy chain comprising the amino acid sequence of SEQ ID NO:33 and a light chain comprising the amino acid sequence of SEQ ID NO:34.
69E. The use of any one of embodiments 1E-68E, wherein the anti-PD-1 antibody is pembrolizumab.
70E. The use of any one of embodiments 1E-69E, wherein the route of administration for the anti-PD-1 antibody or antigen-binding fragment thereof is intravenous or subcutaneous.
71E. The use any one of embodiments 1E-69E, wherein the route of administration for the anti-PD-1 antibody or antigen-binding fragment thereof is intravenous.
72E. The use of any one of embodiments 1E-69E, wherein the route of administration for the anti-PD-1 antibody or antigen-binding fragment thereof is subcutaneous.
73E. The use of any one of embodiments 1E-72E, wherein the anti-PD-1 antibody or antigen-binding fragment thereof and the antibody-drug conjugate are administered sequentially.
74E. The use of any one of embodiments 1E-72E, wherein the anti-PD-1 antibody or antigen-binding fragment thereof and the antibody-drug conjugate are administered simultaneously.
75E. The use of any one of embodiments 1E-74E, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
76E. The use of any one of embodiments 1E-75E, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
77E. The use of any one of embodiments 1E-76E, wherein the subject has a tumor that expresses PD-L1 (TPS≥1).
78E. The use of any one of embodiments 1E-77E, wherein the subject has a tumor that has high PD-L1 expression (TPS≥50).
79E. The use of any one of embodiments 1E-76E, wherein the subject has a tumor that expresses PD-L1 (CPS≥1).
80E. The use of any one of embodiments 1E-79E, wherein a tumor derived from the cancer comprises one or more cells that express PD-L1, PD-L2, or both PD-L1 and PD-L2.
81E. The use of any one of embodiments 1E-80E, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
82E. The use of any one of embodiments 1E-81E, wherein one or more therapeutic effects in the subject is improved after administration of the antibody-drug conjugate and the anti-PD-1 antibody or antigen-binding fragment thereof relative to a baseline.
83E. The use of embodiment 82E, wherein the one or more therapeutic effects is selected from the group consisting of: size of a tumor derived from the cancer, objective response rate, duration of response, time to response, progression free survival, and overall survival.
84E. The use of any one of embodiments 1E-83E, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
85E. The use of any one of embodiments 1E-84E, wherein the objective response rate is at least about 20%, at least about 25%, at least about 30%, at least about 35%, at least about 40%, at least about 45%, at least about 50%, at least about 60%, at least about 70%, or at least about 80%.
86E. The use of any one of embodiments 1E-85E, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
87E. The use of any one of embodiments 1E-86E, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
88E. The use of any one of embodiments 1E-87E, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
89E. The use of any one of embodiments 1E-88E, wherein the subject has one or more adverse events and is further administered an additional therapeutic agent to eliminate or reduce the severity of the one or more adverse events.
90E. The use of any one of embodiments 1E-89E, wherein the subject is at risk of developing one or more adverse events and is further administered an additional therapeutic agent to prevent or reduce the severity of the one or more adverse events.
91E. The use of any one of embodiments 89E-90E, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
92E. The use of any one of embodiments 89E-91E, wherein the one or more adverse events is a grade 3 or greater adverse event.
93E. The use of any one of embodiments 89E-91E, wherein the one or more adverse events is a serious adverse event.
94E. The use of any one of embodiments 89E-91E, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
95E. The use of any one of embodiments 1E-94E, wherein the subject is a human.
96E. The use of any one of embodiments 1E-95E, wherein the antibody-drug conjugate is in a pharmaceutical composition comprising the antibody-drug conjugate and a pharmaceutical acceptable carrier.
97E. The use of any one of embodiments 1E-96E, wherein the anti-PD-1 antibody or antigen-binding fragment thereof is in a pharmaceutical composition comprising the anti-PD-1 antibody or antigen-binding fragment thereof and a pharmaceutical acceptable carrier.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: MMAE Elicits Hallmark Characteristics Associated with Immunogenic Cell Death in a Cervical Cancer Cell Line

Immunogenic cell death (ICD) is a regulated cell death program that is highlighted by the production and exposure of pro-inflammatory signals that leads to the generation of immune responses against the apoptotic tumor cells. ICD is characterized by: 1) exposure of endoplasmic reticulum (ER)-resident chaperone proteins on the surface of tumor cells; 2) secretion of ATP; and 3) secretion of HMGB1. Induction of ER stress is critical for regulating these 3 processes and has been shown to be elicited by antibody-drug conjugates (ADCs) wherein the conjugated drug is MMAE.

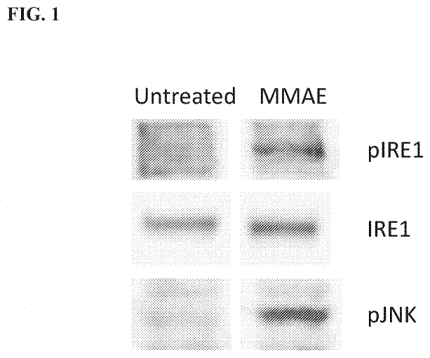

HeLa cells, a cervical cancer cell line, were cultured in Minimum Essential Medium (MEM) with 10% FBS, 10 mM HEPES, 1 mM sodium pyruvate, 2 mM L-glutamine, penicillin (100U/ml), and streptomycin (100 μg/ml). HeLa cells were treated with 100 nM MMAE for 16 hours and harvested in radioimmunoprecipitation assay buffer (RIPA) buffer for western blot analysis. Treatment with MMAE led to phosphorylation of the serine threonine kinase IRE1, indicating activation of ER stress. Severe ER stress is a prerequisite to the exposure of pro-phagocytic signals on the surface of tumor cells, and can be indicated by activation of JNK signaling by phosphorylated IRE1. As demonstrated herein, treatment with MMAE elicited severe ER stress by phosphorylation of IRE1 and JNK (FIG. 1).

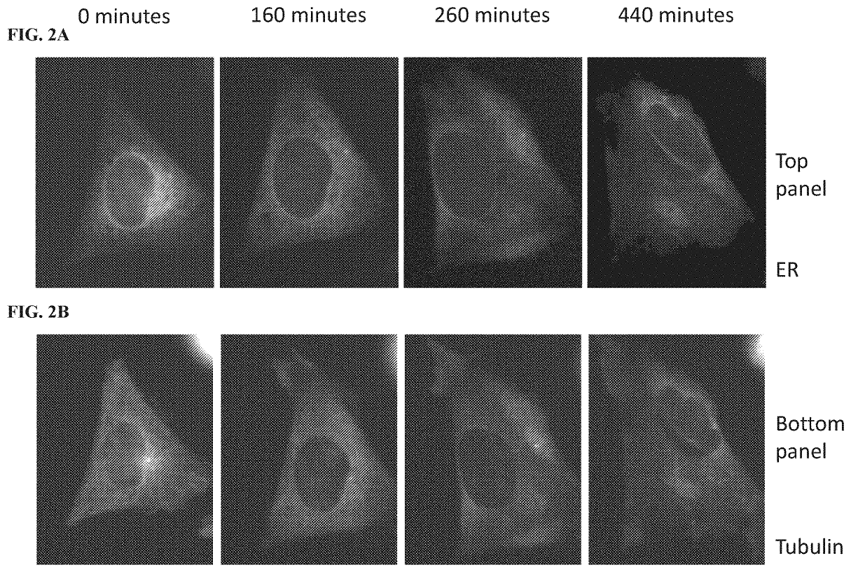

Treatment of HeLa cells with MMAE led to disassembly of the microtubule network and subsequent ER mislocalization. HeLa cells were transduced with a baculovirus encoding RFP-labeled Tubulin (CellLight Tubulin-RFP, ThermoFisher Scientific) and an ER-binding dye (ER-ID Green, Enzo Life Sciences). Cells were treated with 100 nM MMAE and imaged over time in the present of MMAE. Within 2 hours, fragmentation and disassembly of the microtubule network became evident, concurrent with the breakdown of the perinuclear organized ER lattice (FIGS. 2A and 2B). The condensed and mislocalized ER skeleton indicated severe ER stress within 8 hours.

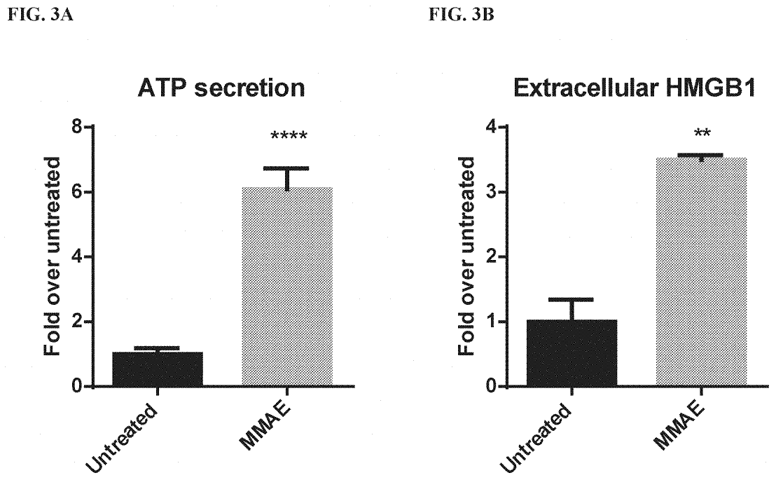

Induction of ICD is also characterized by the secretion of ATP and HMGB1. Extracellular ATP serves as a strong chemotactic signal, promoting immune cell migration to the tumor site. Upon arrival, extracellular HMGB1 signals through various pro-inflammatory receptors (TLR2, TLR4, RAGE) to activate antigen-presenting cells, thereby promoting immune activity within the tumor. As demonstrated herein, treatment of HeLa cells with 100 nM MMAE led to increased secretion of ATP and HMGB1 over a period of 24 hours (FIGS. 3A and 3B; **p<0.01, ****p<0.0001).

While the sequence of events of ADC binding to antigen positive cells, cleavage and release of the MMAE payload, and subsequent cell death is the primary mechanism of tisotumab vedotin functionality, each step in this process can evoke additional and distinct modalities that may contribute to overall antitumor activity. The MMAE cytotoxic payload connected to tisotumab vedotin disrupts microtubules which results in subsequent endoplasmic reticulum (ER) stress that drives exposure of immune activating molecules that can promote a T-cell response. The effect of MMAE on a cervical cancer cell line as shown in this example, demonstrates induction of the ER stress pathway and exposure of immune activating molecules. Accordingly, the T-cell response that may occur following tumor cell death with tisotumab vedotin could amplify the effect of treatment with a checkpoint inhibitor.

### Example 2: Anti-Tumor Activity of Tisotumab Vedotin in Combination with an Anti-PD-1 Monoclonal Antibody in a Xenograft Model in Humanized Mice

Tisotumab vedotin is an antibody-drug conjugate comprising an antibody that binds to tissue factor (TF), a protease-cleavable linker, and the microtubule disrupting agent MMAE. TF is a protein aberrantly expressed in a wide number of tumors including cervical cancer and is associated with poor prognosis. See Forster Y et al. Clin Chim Acta. 2006; 364(1-2):12-21 and Cocco E et al. BMC Cancer. 2011; 11:263. Tisotumab vedotin selectively targets TF to deliver a clinically validated toxic payload to tumor cells. See Breij E C et al. Cancer Res. 2014; 74(4):1214-1226 and Chu A J. Int J Inflam. 2011; 2011. doi: 10.4061/2011/367284.

The anti-PD-1 antibody, pembrolizumab (KEYTRUDA®), is a checkpoint inhibitor that is a standard of care therapy alone or in combination with chemotherapies in multiple tumor indications. The combination of tisotumab vedotin with an anti-PD-1 antibody such as pembrolizumab was evaluated herein for the treatment of cancer.

Materials and Methods

The in vivo anti-tumor efficacy of tisotumab vedotin in combination with an anti-PD-1 monoclonal antibody was evaluated in NOD.Cg-PrkdC^{scid} Il2rg^{tm1Wj1}/SzJ (NSG) immunodeficient mice (The Jackson Laboratory, Stock No. 005557), humanized by engraftment with human CD34⁺ hematopoietic stem cells (Jackson Laboratories, Sacramento). Mice were subcutaneously inoculated with 5×10⁶ MDA-MB-231 cells (breast adenocarcinoma; American Tissue Culture Collection (ATCC), cat. no. HTB-26), in 100 μL phosphate-buffered saline (PBS). Before inoculation, cells were cultured in DMEM with high glucose and HEPES without L-glutamine (Lonza, cat. no. BE12-709F), 10% (v/v) donor bovine serum with iron New Zealand Origin (Thermo Fisher Scientific, DBSI, cat. no. 10371-029), 2 mM L-glutamine (Lonza, cat. no. BE17-605E), 1 mM Na-pyruvate (Lonza, cat. no. BE13-115E), MEM non-essential amino acids (Life Technologies, cat. no. 11140) and 1% (v/v) penicillin/streptomycin (Lonza, cat. no. DE17-603E), in CellSTACK culture chambers (Corning, cat. no. 3313).

Tumor size was determined by caliper measurement at least two times a week and tumor volume was calculated as 0.52×length×width². When tumors reached the size of 100 mm³ mice were randomized in 7 groups (8 mice per treatment group) based on mouse cohort and tumor size (Table 1). Mice were treated with tisotumab vedotin alone (1 mg/kg or 0.5 mg/kg), intravenously, weekly for a maximum of five treatments, or in combination with an anti-PD-1 antibody (i.e., pembrolizumab, KEYTRUDA®, 50 mg concentrate, Merck & Co., Inc., Kenilworth, N.J. USA) or with anti-PD-1 antibody alone. The first dose of the anti-PD-1 antibody (i.e., pembrolizumab) was 10 mg/kg, followed by 5 mg/kg every five days for a maximum of six treatments. Mice in control groups were administered 1 mg/kg of IgG1 isotype control antibody or IgG1 isotype control antibody conjugated to MMAE intravenously, weekly for a maximum of five treatments (Table 1). The IgG1 isotype control antibody is the b12 antibody which is known to bind to HIV-1 gp120. Mice were observed for clinical signs of illness at least twice a week. Mice were housed in individually ventilated (IVC) cages, five mice per cage and identified by ear tags.

TABLE 1
Trial design

| | | Dosing | | Route of | Number of |
|---|---|---|---|---|---|
| Group | Treatment | (mg/kg) | Day of treatment | administration | mice |
| 1 | IgG1 control | 1 mg/kg | Approximately d 14, | IV | 8 |
| | | | d 21, d 28, d 35, | | |
| | | | optionally d 42 | | |
| 2 | IgG1-MMAE | 1 mg/kg | Approximately d 14, | IV | 8 |
| | control | | d 21, d 28, d 35, | | |
| | | | optionally d 42 | | |
| 3 | ADC | 1 mg/kg | Approximately d 14, | IV | 8 |
| | | | d 21, d 28, d 35, | | |
| | | | optionally d 42 | | |
| 4 | PD-1 | First dose | Q5Dx6 | IP | 8 |
| | | 10 mg/kg, | (Approximately d 14, | | |
| | | followed by | d 19, d 24, d 29, d 34, | | |
| | | 5 mg/kg | optionally d 39) | | |
| 5 | ADC + | 1 mg/kg + | Approximately d 14, | IV | 8 |
| | PD-1 | First dose | d 21, d 28, d 35, | | |
| | | 10 mg/kg, | optionally d 42 + | | |
| | | followed by | Q5Dx6 | IP | |
| | | 5 mg/kg | (Approximately d 14, | | |
| | | | d 19, d 24, d 29, d 34, | | |
| | | | optionally d 39) | | |
| 6 | ADC | 0.5 mg/kg | Approximately d 14, | IV | 8 |
| | | | d 21, d 28, d 35, | | |
| | | | optionally d 42 | | |
| 7 | ADC + | 0.5 mg/kg + | Approximately d 14, | IV | 8 |
| | PD-1 | First dose | d 21, d 28, d 35, | | |
| | | 10 mg/kg, | optionally d 42 + | | |
| | | followed by | Q5Dx6 | IP | |
| | | 5 mg/kg | (Approximately d 14, | | |
| | | | d 19, d 24, d 29, d 34, | | |
| | | | optionally d 39) | | |

IgG1 control indicates the IgG1 b12 antibody that binds to HIV-1 gp120 and was used as an IgG1 isotype control; IgG1-MMAE control indicates the IgG1 b12 antibody conjugated to MMAE; ADC indicates anti-IE antibody conjugated to MMAE; and PD-1 indicates anti-PD-1 antibody; IV indicates intravenous administration; and IP indicates intraperitoneal administration.

To determine whether there were statistically significant differences between tumor burden in control and treatment groups, tumor burden in the treatment groups were compared with those in the control groups (e.g., control antibody (e.g., IgG1 control or anti-PD-1 antibody) or control antibody-drug conjugate (e.g., tisotumab vedotin or IgG1-MMAE)). Statistical comparison of tumor burden was performed using Mann-Whitney analysis on the last day that all treatment groups were intact. Kaplan-Meier analysis was performed based on tumor volume (>500 mm³).

Results

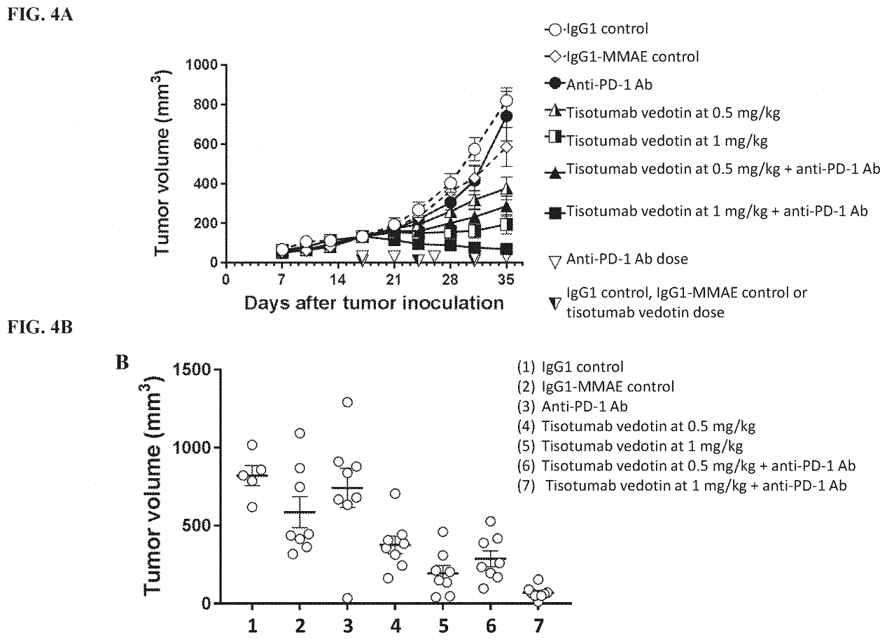

Treatment with pembrolizumab alone hardly reduced tumor burden as assessed by tumor volume (FIGS. 4A and 4B). Treatment with tisotumab vedotin efficiently reduced tumor burden at a dose of 0.5 mg/kg and at a dose of 1.0 mg/kg (FIGS. 4A and 4B). Combination treatment with tisotumab vedotin and pembrolizumab enhanced the induction of tumor regression (FIGS. 4A and 4B).

### Example 3: Anti-Tumor Activity of Tisotumab Vedotin in Combination with an Anti-PD-1 Monoclonal Antibody in a Patient-Derived Xenograft Model in Humanized Mice

Pembrolizumab has been tested in patients with cervical cancer. Pembrolizumab 200 mg Q3W was administered to 82 patients with previously treated, advanced cervical cancer. The objective response rate was 12%. See Schellens J. H. M, et al., J Clin Oncol, 2017, 35. (Suppl.): abstr 5514. The combination of tisotumab vedotin with an anti-PD-1 antibody such as pembrolizumab is evaluated herein for the treatment of cervical cancer.

Materials and Methods

The in vivo anti-tumor efficacy of tisotumab vedotin in combination with an anti-PD-1 monoclonal antibody is evaluated in an animal model such as in NOD.Cg-Prkdc^{scid} Il2rg^{tm1Wj1} (NSG) immunodeficient mice or NOD-Prkdc^{em26Cd52}Il2rg^{emCd22} (NCG) immunodeficient mice humanized by engraftment with human CD34⁺ hematopoietic stem cells. Patient-derived xenografts (PDX) are derived from tumor specimens from cancer patients. Establishment and characterization of the PDX model is performed following the primary implantation into nude mice. Tumor xenografts are passaged approximately three to five times until establishment of stable growth patterns. Tumor fragments are obtained from xenografts in serial passage in nude mice. Tumors are cut into fragments of 4-5 mm diameter and placed in phosphate-buffered saline (PBS) until subcutaneous implantation. Cervical cancer PDX models (HUPRIME® cervical xenograft model CV1802 and CV2302; Crown Bioscience Inc.) are used in this experiment. Tumor size is determined by caliper measurement at least two times a week and tumor volume is calculated as 0.52×length×width². When tumors reach the volume of 150-250 mm³, mice are randomized in 7 groups per model (10 mice per treatment group), based on tumor volume. Mice are treated with intravenous injections of tisotumab vedotin alone (e.g., at two dose levels between 0.5 mg/kg and 4 mg/kg, weekly) or in combination with an anti-PD-1 monoclonal antibody (e.g., pembrolizumab, KEYTRUDA®; at a dose between 5 and 15 mg/kg, every 5-7 days) or with anti-PD-1 antibody alone (e.g., pembrolizumab, KEYTRUDA®; at a dose between 5 and 15 mg/kg, every 5-7 days). In one example, when the HUPRIME® cervical xenograft model CV2320 is used, mice are treated with intravenous injections of tisotumab vedotin alone at a dose of 4 mg/kg or 2 mg/kg, or in combination with an anti-PD-1 monoclonal antibody (e.g., pembrolizumab) at a first dose of 10 mg/kg, followed by a 5 mg/kg dose every 5 days until a maximum amount of treatment is reached (e.g., five treatments). HUPRIME® cervical xenograft model CV2320 treated with anti-PD-1 monoclonal antibody alone (e.g., pembrolizumab) are provided at a first dose of 10 mg/kg, followed by a 5 mg/kg dose every 5 days until a maximum amount of treatment is reached (e.g., five treatments). In another example, when the HUPRIME® cervical xenograft model CV1802 is used, mice are treated with intravenous injections of tisotumab vedotin alone at a dose of 1 mg/kg or 0.5 mg/kg, or in combination with an anti-PD-1 monoclonal antibody (e.g., pembrolizumab) at a first dose of 10 mg/kg, followed by a 5 mg/kg dose every 5 days until a maximum amount of treatment is reached (e.g., five treatments). HUPRIME® cervical xenograft model CV1802 treated with anti-PD-1 monoclonal antibody alone (e.g., pembrolizumab) are provided aa first dose of 10 mg/kg, followed by a 5 mg/kg dose every 5 days until a maximum amount of treatment is reached (e.g., five treatments). Mice are observed for clinical signs of illness at least twice a week. Mice are housed in individually ventilated (IVC) cages, five mice per cage and identified by ear tags.

To determine whether there are statistically significant differences between tumor volumes in control and treatment groups, tumor volumes in the treatment groups are compared with those in the control groups (e.g., control antibody (e.g., IgG1 control or anti-PD-1 antibody) or control antibody-drug conjugate (e.g., tisotumab vedotin or IgG1-MMAE)), using Mann-Whitney analysis at the last day that all groups are intact. Tumor volumes in mice treated with both tisotumab vedotin and anti-PD-1 antibody are compared with those in mice treated with either control antibody alone (e.g., IgG1 control or anti-PD-1 antibody) or control antibody-drug conjugate alone (e.g., tisotumab vedotin or IgG1-MMAE) and analyzed such as by using Mantel-Cox analysis on Kaplan-Meier plots.

### Example 4: Anti-Tumor Activity of Tisotumab Vedotin in Combination with an Anti-PD-1 Monoclonal Antibody in a Syngeneic Tumor Model

Mouse tumor cells are transfected with plasmid constructs encoding human tissue factor (TF) and sgRNA-guided Cas9 nuclease (sgRNA/Cas9) to generate a murine cell line that expresses the human TF. Fluorescence activated cell sorting (FACS) yields a clonal population of murine tumor cells that stably express human TF, these cells are then treated with 1 μg to 5 μg per ml of tisotumab vedotin or 100 nM of MMAE for 4 days. In order to prepare dying cells for immunization, treated murine tumor cells are overlaid atop Histopaque, and centrifuged at 2000 g for 30 minutes. Dead and dying cells are pelleted underneath the Histopaque layer, and viability assessed by trypan blue exclusion. A sample with approximately <20% live cells as measured by trypan blue exclusion is obtained. Flash-frozen tumor cells are prepared by submerging the cells in liquid nitrogen for 10 seconds, followed by immersion in 37° C. water until completely thawed. The liquid nitrogen freeze-thaw process is repeated 5 times. Dead and dying human TF positive tumor cells are resuspended in phosphate buffered saline (PBS) and 2×10⁶ cells are injected into the peritoneum of immune-competent Balb/c mice. Seven days later, mice receive a second immunization with dead and dying cells prepared in the same manner.

Fourteen days after initial immunization with the dead and dying human TF positive tumor cells, mice are subcutaneously implanted with 5×10⁶ wild-type tumor cells and monitored for tumor growth. Mice that are immunized with tisotumab vedotin-killed tumor cells or MMAE-killed tumor cells experience delayed tumor growth and increased survival. As these effects occur in the absence of any administered therapeutic agent, the administration of cells killed by tisotumab vedotin or MMAE is sufficient to generate long-lasting protective immune memory against subsequent tumor cell challenge. Protective immune memory is amplified by treating these mice with tisotumab vedotin in combination with an antibody that binds to murine PD-1. This combination treatment increases the number of mice that are cured by subsequent tumor challenge.

### Example 5: A Phase II Trial of Tisotumab Vedotin Alone or in Combination with a Monoclonal Anti-PD-1 Antibody in First Line Recurrent or Stage IVB Cervical Cancer

A Phase I/II trial demonstrated a robust efficacy and manageable safety profile for 2.0 mg/kg tisotumab vedotin administered to subjects with relapsed, recurrent, and/or metastatic cervical cancer (NCT02001623). That preliminary data suggests a positive benefit risk profile for that population of high unmet need. Further investigation of tisotumab vedotin as a monotherapy and in combination with immunotherapy (e.g., an anti-PD-1 antibody) in a larger cohort of patients with cervical cancer is needed.

The efficacy, safety and tolerability of 0.9 mg/kg, 1.2 mg/kg, 1.3 mg/kg or 2.0 mg/kg tisotumab vedotin alone or in combination with pembrolizumab, a monoclonal anti-PD-1 antibody, in subjects with first line recurrent or Stage IVB cervical cancer is evaluated herein.

Methods

This phase II, open-label, multi-center trial evaluates the efficacy, safety and tolerability of tisotumab vedotin alone or in combination with the anti-PD-1 antibody, pembrolizumab, in subjects with first line recurrent or Stage IVB squamous, adenosquamous, or adenocarcinoma of the cervix who are not amenable to curative treatment with surgery and/or radiation therapy and who have not received prior systemic therapy for their recurrent or Stage IVB disease. Subjects with recurrent disease who are candidates for curative therapy by means of pelvic exenteration are not eligible to participate in the trial.

Subjects are symmetrically allocated to one of six treatment groups. The allocation is done in a way that minimizes imbalance on disease status (metastatic/recurrent) and histology (squamous/non-squamous). Eligible subjects are treated with tisotumab vedotin 1.3 mg/kg Q3W, tisotumab vedotin 2.0 mg/kg Q3W, tisotumab vedotin 0.9 mg/kg 3Q4W+pembrolizumab 200 mg Q3W, tisotumab vedotin 1.2 mg/kg 3Q4W+pembrolizumab 200 mg Q3W, tisotumab vedotin 1.3 mg/kg Q3W+pembrolizumab 200 mg Q3W or tisotumab vedotin 2.0 mg/kg Q3W+pembrolizumab 200 mg Q3W. Treatment cycles occur every 21 days (±3 days) for Q3W treatment cycles or every 28 days (+4 days) for 3Q4W treatment cycles. All treatment components are administered intravenously (IV). Approximately 60 subjects, age ≥18 years, are enrolled in the trial. The duration of the trial is approximately 7 years. Inclusion criteria and exclusion criteria for subjects enrolled in the trial are shown in Table 2.

TABLE 2
List of inclusion and exclusion criteria

| | |
|---|---|
| Inclusion Criteria | Must have recurrent or Stage IVB squamous, adenosquamous, |
| | or adenocarcinoma histologies of the cervix which are not |
| | amenable to curative treatment with surgery and/or radiation |
| | therapy. |
| | Must have not received prior systemic therapy for recurrent or |
| | Stage IVB disease. |
| | Note: Subjects are excluded if they are candidates for curative |
| | therapy by means of pelvic exenteration. |
| | Note: Chemotherapy administered in the adjuvant or |
| | neoadjuvant setting, or in combination with radiation therapy is |
| | not counted as a prior systemic therapy. |
| | Must have baseline measurable disease per RECIST v1.1. |
| | Note: Lesions situated in a previously irradiated area are |
| | considered measurable if progression has been demonstrated in |
| | such lesions. |
| | Age ≥18 years of age on day of signing informed consent. |
| | Acceptable renal function: Calculated (Cockcroft-Gault) |
| | Glomerular Filtration Rate (GFR) > 50 mL/min. |
| | Acceptable liver function: |
| | Alanine aminotransferase (ALT) and aspartate |
| | aminotransferase (AST) ≤ 2.5 × Upper Limit of Normal |
| | (ULN) (if liver tumor/metastases are present, then ≤5 × ULN |
| | is allowed); |
| | Bilirubin ≤ 1.5 × ULN unless direct bilirubin ≤ institutional |
| | ULN, except in subjects diagnosed with Gilbert's syndrome, |
| | direct bilirubin ≤ 2 × ULN. |
| | Acceptable hematological status: |
| | Hemoglobin ≥ 5.6 mmol/L (9.0 g/dL).* |
| | Absolute neutrophil count (ANC) ≥ 1500/μL (1.5 × 10⁹/L). |
| | Platelet count ≥ 100 × 10⁹/L. |
| | *Acceptable hematologic status must be met without |
| | erythropoietin dependency and without packed red blood cell |
| | (pRBC) transfusion within the last 2 weeks. |
| | Acceptable coagulation status: |
| | For subjects not on anti-coagulation therapy: |
| | Activated partial thromboplastin time (aPTT) ≤ |
| | 1.25 × ULN. |
| | International normalized ratio (INR) ≤ 1.2. |
| | For subjects on anti-coagulation therapy: |
| | aPTT ≤ 1.25 × ULN |
| | INR: (1) Subjects on anti-coagulation therapy requiring |
| | laboratory assessments for dose titration (warfarin or other |
| | Vitamin K dependent anticoagulant agents) must be on a |
| | steady dose (no active titration) for at least 4 weeks prior to |
| | first planned dose of tisotumab vedotin and must have an INR ≤ |
| | 2.5 for eligibility. (2) Subjects on anti-coagulants that do |
| | not require laboratory assessments for dose titration must |
| | have an INR of ≤1.2 and do not need to be on a stable dose |
| | for ≥4 weeks prior to first planned dose of IMP. |
| | Concurrent use of prophylactic Acetyl Salicylic Acid (ASA, |
| | e.g., aspirin) is prohibited for subjects on any type of anti- |
| | coagulation therapy. |
| | Eastern Cooperative Oncology Group (ECOG) performance |
| | status of 0 or 1. |
| | Life expectancy of ≥3 months |
| | A female subject is eligible to participate if she is not pregnant, |
| | breastfeeding, or expecting to conceive children, or expecting |
| | to donate eggs for the purposes of assisted reproduction within |
| | the projected duration of the trial and for at least 6 months after |
| | the last trial administration and at least one of the following |
| | conditions applies: |
| | Not a woman of childbearing potential (WOCBP) |
| | A WOCBP must agree to use adequate contraception during and |
| | for 6 months after the last dose of trial treatment administration. |
| | Adequate contraception for women is defined as highly effective |
| | methods of contraception. In countries where two highly |
| | effective methods of contraception are required this will be an |
| | inclusion criterion. |
| | Must provide a fresh specimen from a lesion not previously |
| | irradiated. Subjects for whom fresh samples cannot be obtained |
| | (e.g., inaccessible tumor or for safety concerns) may submit an |
| | archived specimen in place of the fresh tissue. |
| | Note: Aspirates are not acceptable. |
| | Must have recovered from all AEs due to previous therapies to ≤ |
| | grade 1. Subjects with ≤ grade 2 neuropathy or alopecia are |
| | eligible. |
| | Must be willing and able to adhere to the prohibitions and |
| | restrictions specified in this protocol. |
| | Following receipt of verbal and written information about the |
| | trial, subjects must provide signed informed consent before any |
| | trial-related activity is carried out. |
| Exclusion Criteria | Clinically relevant bilateral hydronephrosis which cannot be |
| | alleviated by ureteral stents or percutaneous drainage. |
| | Have clinical signs or symptoms of gastrointestinal obstruction |
| | and requires parenteral hydration and/or nutrition. |
| | Hematological: Known past or current coagulation defects |
| | leading to an increased risk of bleeding; diffuse alveolar |
| | hemorrhage from vasculitis; known bleeding diathesis; ongoing |
| | major bleeding; trauma with increased risk of life-threatening |
| | bleeding or history of severe head trauma or intracranial surgery |
| | within 8 weeks of trial entry. |
| | Ophthalmological: Active ocular surface disease at baseline. |
| | Subjects with prior history of cicatricial conjunctivitis or Steven |
| | Johnson Syndrome are not eligible to participate. |
| | Has an active autoimmune disease that has required systemic |
| | treatment in past 2 years (i.e., with use of disease modifying |
| | agents, corticosteroids or immunosuppressive drugs). |
| | Replacement therapy (e.g., thyroxine, insulin, or physiologic |
| | corticosteroid replacement therapy for adrenal or pituitary |
| | insufficiency) is not considered a form of systemic treatment and |
| | is allowed. |
| | Cardiovascular: Clinically significant cardiac disease including |
| | unstable angina, acute myocardial infarction within 6 months |
| | prior to screening; any medical history of congestive heart failure |
| | (Grade III or IV as classified by the New York Heart |
| | Association), any medical history of decreased cardiac ejection |
| | fraction of <45%; a marked baseline prolongation of QT/QTc |
| | interval (e.g., repeated demonstration of a QTc interval > 450 |
| | msec), a complete left bundle branch block (defined as a QRS |
| | interval ≥ 120 msec in left bundle branch block form) or an |
| | incomplete left bundle branch block. |
| | Current or a prior history of (non-infectious) pneumonitis that |
| | required steroids or has current pneumonitis. |
| | Other cancers: Known past or current malignancy other than |
| | inclusion diagnosis, except for: Non-invasive basal cell or |
| | squamous cell skin carcinoma; noninvasive, superficial bladder |
| | cancer; any cancer with a complete response (CR) of >5 years |
| | duration. |
| | Known active CNS metastases and/or carcinomatous meningitis. |
| | Subjects with previously treated brain metastases may participate |
| | provided they are radiologically stable, (i.e., without evidence of |
| | progression) for at least 28 days by repeat imaging (note that the |
| | repeat imaging should be performed during trial screening), |
| | subjects should be clinically stable, and should not require steroid |
| | treatment for at least 14 days prior to first dose of trial treatment. |
| | Prior therapy: |
| | Any prior treatment with MMAE-derived drugs. |
| | Has received prior para-aortic radiation. |
| | Prior radiotherapy (with the exception of para-aortic |
| | radiation) within 2 weeks (14 days) of start of trial treatment. |
| | Subjects must have recovered from all radiation-related |
| | toxicities, not require corticosteroids, and not have had |
| | radiation pneumonitis. A 1-week washout is permitted for |
| | palliative radiation (≤2 weeks of radiotherapy) to non-CNS |
| | disease. |
| | Has received prior systemic anti-cancer therapy including |
| | investigational agents within 4 weeks (28 days) prior to the |
| | first dose of trial treatment. |
| | Has received prior therapy with an anti-PD-1, anti-PD-L1, or |
| | anti PD-L2 agent or with an agent directed to another |
| | stimulatory or co-inhibitory T-cell receptor (e.g., CTLA-4, |
| | OX40, CD137) and was discontinued from treatment due to |
| | a grade 3 or higher AE of special interest (AESI). |
| | Surgery/procedures: major surgery within 4 weeks (28 days) or |
| | minor surgery within 7 days prior to the first dose of trial |
| | treatment. Subjects must have recovered adequately from the |
| | toxicity and/or complications from the intervention prior to |
| | starting trial treatment. Subjects who have planned major surgery |
| | during the treatment period must also be excluded from the trial. |
| | Has a diagnosis of immunodeficiency or is receiving systemic |
| | steroid therapy (in dosing exceeding 10 mg daily of prednisone |
| | or equivalent) or any other form of immunosuppressive therapy |
| | within 7 days prior to the first dose of trial treatment. |
| | Received a live vaccine within 30 days prior to the first dose of |
| | trial treatment. Examples of live vaccines include, but are not |
| | limited to, the following: measles, mumps, rubella, |
| | varicella/zoster (chicken pox), yellow fever, rabies, Bacillus |
| | Calmette-Gúerin, and typhoid vaccine. Seasonal influenza |
| | vaccines for injection are generally killed virus vaccines and are |
| | allowed; however, intranasal influenza vaccines (e.g., FluMist ®) |
| | are live attenuated vaccines and are not allowed. |
| | Is currently participating in or has participated in a trial of an |
| | investigational agent or has used an investigational device within |
| | 4 weeks prior to the first dose of trial treatment. |
| | Note: Subjects who have entered the follow-up phase of an |
| | investigational trial may participate as long as it has been 4 |
| | weeks after the last dose of the previous investigational agent. |
| | Other: Ongoing significant, uncontrolled medical condition; |
| | clinically significant active viral, bacterial or fungal infection |
| | requiring IV or oral (PO) treatment with antimicrobial therapy |
| | ending less than 7 days prior to first trial treatment |
| | administration; |
| | Known history of human immunodeficiency virus (HIV) |
| | infection. No HIV testing is required unless mandated by local |
| | health authority. |
| | Known history of Hepatitis B (defined as Hepatitis B surface |
| | [HBsAg] reactive) or known active Hepatitis C virus (defined as |
| | HCV RNA [qualitative] is detected) infection. |
| | Note: No testing for Hepatitis B and Hepatitis C is required |
| | unless mandated by local health authority. |
| | Has known allergies, severe hypersensitivity (≥Grade 3), or |
| | intolerance to tisotumab vedotin, pembrolizumab, or their |
| | excipients. |
| | Has a history or current evidence of any condition, therapy, or |
| | laboratory abnormality that might confound the results of the |
| | trial, interfere with the subject's participation for the full duration |
| | of the trial, or is not in the best interest of the subject to |
| | participate, in the opinion of the treating investigator. |
| | Has known psychiatric or substance abuse disorders that would |
| | interfere with cooperating with the requirements of the trial. |
| | A WOCBP who has a positive pregnancy test (e.g., within 72 |
| | hours) prior to treatment. If the urine test is positive or cannot be |
| | confirmed as negative, a serum pregnancy test will be required. |
| | Subjects that are postmenopausal or permanently sterilized can |
| | be considered as not having reproductive potential. |

Lyophilized vials containing 40 mg of tisotumab vedotin are stored in a refrigerator at 2° C. to 8° C. Tisotumab vedotin is reconstituted in 4 ml of water leading to a reconstituted solution comprising 10 mg/mL tisotumab vedotin, 30 mM histidine, 88 mM sucrose, and 165 mM D-mannitol. The reconstituted antibody drug-conjugate solution has a pH of 6.0. The reconstituted tisotumab vedotin is diluted into a 0.9% NaCl 100 mL infusion bag according to the dose calculated for the subject. Intravenous infusion is completed within 24 hours after the tisotumab vedotin vial has been reconstituted. A 0.2 μm in-line filter is used for the intravenous infusion. The entire 100 mL volume from the prepared infusion bag is administered. No dead volume is provided. Pembrolizumab (KEYTRUDA®) injection is a sterile, preservative-free, clear to slightly opalescent, colorless to slightly yellow solution that requires dilution for intravenous infusion. Each vial contains 100 mg of pembrolizumab in 4 mL of solution. Each 1 mL of solution contains 25 mg of pembrolizumab and is formulated in: L-histidine (1.55 mg), polysorbate 80 (0.2 mg), sucrose (70 mg), and Water for Injection, USP. The dose of pembrolizumab for administration to the subject is calculated at the trial site.

Objectives and endpoints are described in Table 3. Subjects are treated until disease progression, toxicity, or withdrawal of consent. For subjects participating in the tisotumab vedotin in combination with pembrolizumab treatment group, treatment with pembrolizumab is discontinued after the subject has completed 35 treatments (approximately 2 years) with pembrolizumab. Treatment with pembrolizumab may also be discontinued if the subject achieves a confirmed complete response (CR) and has been treated for at least 8 cycles (≥24 weeks) and the subject has received at least 2 doses of pembrolizumab beyond the date when the initial CR was declared. Subjects may continue to receive tisotumab vedotin monotherapy after the discontinuation of pembrolizumab if the subject has achieved stable disease (SD) or better.

Imaging is obtained every 6 weeks for 32 weeks and then every 12 weeks thereafter, calculated from the date of first dose. On-trial imaging is continued until the subject experiences radiographic disease progression, begins a new anti-cancer therapy, withdraws consent or subject death. Tumor response is analyzed at three time points; futility assessment, early efficacy assessment, and primary efficacy assessment, respectively.

TABLE 3
Objectives and endpoints

| | |
|---|---|
| OBJECTIVES | ENDPOINTS |

Primary

| | |
|---|---|
| Evaluate anti-tumor efficacy of tisotumab | Objective Response Rate (ORR) as |
| vedotin alone or in combination with | determined per Response Evaluation |
| pembrolizumab. | Criteria in Solid Tumors (RECIST) v1.1. |

Secondary

| | |
|---|---|
| Assess safety and tolerability of | Frequency, duration, and severity of |
| tisotumab vedotin alone or in | adverse events (AEs) and evaluation of |
| combination with pembrolizumab. | safety laboratory parameters. |
| Evaluate durability of tisotumab vedotin | Duration of Response (DOR) per |
| alone or in combination with | RECIST v1.1. |
| pembrolizumab. | Time to Response (TTR) per RECIST |
| | v1.1. |
| Evaluate clinical response with tisotumab | Progression free survival (PFS) per |
| alone or in combination with | RECIST v 1.1. |
| pembrolizumab. | Overall Survival (OS). |
| To evaluate the pharmacokinetics (PK) | TPK and anti-drug antibodies (ADA) |
| and immunogenicity of tisotumab | associated with tisotumab vedotin alone |
| vedotin alone and in combination with | and in combination with pembrolizumab. |
| pembrolizumab. | |

Exploratory

| | |
|---|---|
| Explore relationship between biomarkers | TF and PD-L1 expression in tumor |
| and clinical response. | biopsies, circulating TF, proteomic |
| | analyses and genomic signatures. |
| Assess potential pharmacodynamic | Circulating tissue factor (TF) and |
| biomarkers. | proteomic analyses. |

For subjects that do not tolerate the protocol-specified dosing schedule, dose reductions are permitted for tisotumab vedotin in order to allow the subject to continue treatment with tisotumab vedotin alone or in combination with pembrolizumab (Table 4).

TABLE 4
Dose modification scheme for tisotumab vedotin

| Current Dose of | Reduced Dose of |
|---|---|
| Tisotumab Vedotin | Tisotumab Vedotin |
| 0.9 mg/kg | 0.65 mg/kg |
| 1.2 mg/kg | 0.9 mg/kg* |
| 1.3 mg/kg | 0.9 mg/kg* |
| 2.0 mg/kg | 1.3 mg/kg |

*No more than 2 dose reductions of tisotumab vedotin will be permitted. If an AE recurs after the second dose reduction of tisotumab vedotin, then the subject must be permanently discontinued from trial treatment.

The dose of pembrolizumab cannot be reduced but may be held. AEs associated with pembrolizumab exposure may represent an immunologic etiology. These immune-related AEs (irAEs) may occur shortly after the first dose or several months after the last dose of pembrolizumab treatment and may affect more than one body system simultaneously. Based on existing clinical trial data, most irAEs were reversible and could be managed with interruptions of pembrolizumab, administration of corticosteroids and/or other supportive care. Based on the severity of irAEs, withhold or permanently discontinue pembrolizumab and administer corticosteroids. Dose modification and toxicity management guidelines for irAEs associated with pembrolizumab are provided in Table 5. Corticosteroid taper should be initiated upon AE improving to Grade 1 or less and continue to taper over at least 4 weeks. For situations where pembrolizumab and tisotumab vedotin are withheld, pembrolizumab can be resumed after AE has been reduced to Grade 1 or 0 and corticosteroid has been tapered. Tisotumab vedotin can be resumed after the AE has been reduced to Grade 1 or 0. Pembrolizumab and tisotumab vedotin should be permanently discontinued if AE does not resolve within 12 weeks of last dose or corticosteroids cannot be reduced to 10 mg prednisone or equivalent per day within 12 weeks. Pembrolizumab should be discontinued for any recurrent ≥grade 3 irAE that recurs. For severe and life-threatening immune-related adverse events (irAEs), IV corticosteroid should be initiated first followed by oral steroid. Other immunosuppressive treatment should be initiated if irAEs cannot be controlled by corticosteroids.

TABLE 5
Dose modification and toxicity management guidelines for irAEs associated with
the pembrolizumab in combination with tisotumab vedotin treatment group

| | | | | irAE management with | |
|---|---|---|---|---|---|
| Immune- | Toxicity grade or | Action taken to | Action taken to | corticosteroid and/or | Monitor and |
| related AEs | conditions (CTCAE v4.0) | pembrolizumab | tisotumab vedotin | other therapies | follow-up |
| Pneumonitis | Grade 2 | Withhold | Withhold | Administer | Monitor |
| | Grade 3 or 4, | Permanently | Withhold¹ | corticosteroids | subjects for |
| | or recurrent ≥ | discontinue | | (initial dose of 1-2 | signs and |
| | Grade 2 | | | mg/kg prednisone | symptoms of |
| | | | | or equivalent) | pneumonitis |
| | | | | followed by taper | Evaluate |
| | | | | | subjects with |
| | | | | | suspected |
| | | | | | pneumonitis |
| | | | | | with |
| | | | | | radiographic |
| | | | | | imaging and |
| | | | | | initiate |
| | | | | | corticosteroid |
| | | | | | treatment |
| | | | | | Add |
| | | | | | prophylactic |
| | | | | | antibiotics for |
| | | | | | opportunistic |
| | | | | | infections |
| Diarrhea/ | Grade 2 or 3 | Withhold | Withhold | Administer | Monitor |
| Colitis | | | | corticosteroids | subjects for |
| | | | | (initial dose of 1-2 | signs and |
| | | | | mg/kg prednisone | symptoms of |
| | | | | or equivalent) | enterocolitis |
| | | | | followed by taper | (i.e., diarrhea, |
| | Grade 4 | Permanently | Withhold² | | abdominal pain, |
| | | discontinue | | | blood or mucus |
| | | | | | in stool with or |
| | | | | | without fever) |
| | | | | | and of bowel |
| | | | | | perforation |
| | | | | | (i.e., peritoneal |
| | | | | | signs and ileus). |
| | | | | | Subjects with ≥ |
| | | | | | Grade 2 |
| | | | | | diarrhea |
| | | | | | suspecting |
| | | | | | colitis should |
| | | | | | consider GI |
| | | | | | consultation |
| | | | | | and performing |
| | | | | | endoscopy to |
| | | | | | rule out colitis. |
| | | | | | Subjects with |
| | | | | | diarrhea/colitis |
| | | | | | should be |
| | | | | | advised to drink |
| | | | | | liberal |
| | | | | | quantities of |
| | | | | | clear fluids. If |
| | | | | | sufficient oral |
| | | | | | fluid intake is |
| | | | | | not feasible, |
| | | | | | fluid and |
| | | | | | electrolytes |
| | | | | | should be |
| | | | | | substituted via |
| | | | | | IV infusion. |
| AST/ALT | Grade 2 | Withhold | Withhold | Administer | Monitor with |
| elevation or | | | | corticosteroids | liver function |
| Increased | | | | (initial dose of | tests (consider |
| bilirubin | | | | 0.5-1 mg/kg | weekly or more |
| | | | | prednisone or | frequently until |
| | | | | equivalent) | liver enzyme |
| | | | | followed by taper | value returned |
| | Grade 3 or 4 | Permanently | Withhold² | Administer | to baseline or is |
| | | discontinue | | corticosteroids | stable |
| | | | | (initial dose of 1-2 | |
| | | | | mg/kg prednisone | |
| | | | | or equivalent) | |
| | | | | followed by taper | |
| Type 1 | Newly onset | Withhold³ | Withhold | Initiate insulin | Monitor |
| diabetes | T1DM or | | | replacement | subjects for |
| mellitus | Grade 3 or 4 | | | therapy for | hyperglycemia |
| (T1DM) or | hyperglycemia | | | subjects with | or other signs |
| Hyperglycemia | associated | | | T1DM | and symptoms |
| | with evidence | | | Administer anti- | of diabetes. |
| | of β-cell | | | hyperglycemic in | |
| | failure | | | subjects with | |
| | | | | hyperglycemia | |
| Hypophysitis | Grade 2 | Withhold | Withhold | Administer | Monitor for |
| | Grade 3 or 4 | Withhold or | Withhold³ | corticosteroids | signs and |
| | | permanently | | and initiate | symptoms of |
| | | discontinue³ | | hormonal | hypophysitis |
| | | | | replacements as | (including |
| | | | | clinically | hypopituitarism |
| | | | | indicated. | and adrenal |
| | | | | | insufficiency). |
| Hyperthyroidism | Grade 2 | Continue | Continue | Treat with non- | Monitor for |
| | Grade 3 or 4 | Withhold or | Withhold³ | selective beta- | signs and |
| | | permanently | | blockers (e.g., | symptoms of |
| | | discontinue³ | | propranolol) or | thyroid |
| | | | | thionamides as | disorders. |
| | | | | appropriate | |
| Hypothyroidism | Grade 2-4 | Continue | Continue | Initiate thyroid | Monitor for |
| | | | | replacement | signs and |
| | | | | hormones (e.g., | symptoms of |
| | | | | levothyroxine or | thyroid |
| | | | | liothyroinine) per | disorders. |
| | | | | standard of care | |
| Nephritis and | Grade 2 | Withhold | Withhold | Administer | Monitor |
| Renal | Grade 3 or 4 | Permanently | Withhold² | corticosteroids | changes of renal |
| dysfunction | | discontinue | | (prednisone 1-2 | function |
| | | | | mg/kg or | |
| | | | | equivalent) | |
| | | | | followed by taper. | |
| Myocarditis | Grade 1 or 2 | Withhold | Withhold | Based on severity | Ensure |
| | Grade 3 or 4 | Permanently | Withhold² | of AE administer | adequate |
| | | discontinue | | corticosteroids | evaluation to |
| | | | | | confirm |
| | | | | | etiology and/or |
| | | | | | exclude other |
| | | | | | causes |
| All other | Intolerable/ | Withhold | Withhold | Based on type and | Ensure |
| immune- | persistent | | | severity of AE | adequate |
| related AEs | Grade 2 | | | administer | evaluation to |
| | Grade 3 | Withhold or | Withhold² | corticosteroids | confirm |
| | | discontinue | | | etiology and/or |
| | | based on the | | | exclude other |
| | | type of event. | | | causes |
| | | Events that | | | |
| | | require | | | |
| | | discontinuation | | | |
| | | include and not | | | |
| | | limited to: | | | |
| | | Gullain-Barre | | | |
| | | Syndrome, | | | |
| | | encephalitis | | | |
| | Grade 4 or | Permanently | Permanently | | |
| | recurrent | discontinue | discontinue | | |
| | Grade 3 | | | | |

Note:
Withhold or permanently discontinue pembrolizumab and tisotumab vedotin is at the discretion of the investigator or treating physician.
¹For grade 3 pneumonitis, tisotumab vedotin monotherapy can continue after consultation with the sponsor if the event resolves to grade 1 or 0 within 12 weeks from the last dose. If pneumonitis recurs, tisotumab vedotin must be discontinued immediately. For grade 4 pneumonitis, tisotumab vedotin must be discontinued immediately.
²Tisotumab vedotin should be withheld until etiology has been established. If the grade 3 event is clearly not related to tisotumab vedotin and if the event resolves to grade 1 or 0 within 12 weeks from the last dose, monotherapy tisotumab may continue after consultation with the sponsor. If the ≥ grade 3 event recurs, tisotumab vedotin must be discontinued immediately. For grade 4 events, discontinue tisotumab vedotin immediately.
³For subjects with grade 3 or 4 immune-related endocrinopathy where withhold of pembrolizumab and tisotumab vedotin is required, pembrolizumab and tisotumab vedotin may be resumed when AE resolves to ≤ grade 2 and is controlled with hormonal replacement therapy or achieved metabolic control (in case of Type 1 diabetes mellitus [T1DM]).

Three adverse events of special interest were identified during treatment with tisotumab vedotin alone in the Phase IIa trial discussed above: 1) ocular adverse events; 2) adverse events of peripheral neuropathy; and 3) adverse events of bleeding. For ocular AEs: AEs of grade 1-2 conjunctivitis were frequently reported in relation to treatment with tisotumab vedotin. Implementation of a comprehensive mitigation plan and preventive measures substantially reduced both the frequency and severity of ocular adverse events. In the present trial, in order to prevent ocular AEs, all subjects in both treatment groups (i.e., tisotumab vedotin alone or in combination with pembrolizumab) must adhere to the following ocular pre-medication guidelines: 1) use of preservative-free lubricating eye drops during the whole treatment phase of the trial (i.e., from first dose of tisotumab vedotin until the safety follow-up visit). Lubricating eye drops should be administered according to the product prescribing information; 2) it is recommended not to wear contact lenses while treated with tisotumab vedotin from the first dose until a safety follow-up visit; 3) use of refrigerator-based eye cooling pads during infusion, e.g. THERA PEARL Eye Mask or similar, to be applied immediately before infusion in accordance with the instructions provided with the eye cooling pads; 4) administration of local ocular vasoconstrictor before infusion (brimonidine tartrate 0.2% eye drops or similar, 3 drops in each eye immediately prior to start of infusion; otherwise to be used in accordance with the product prescribing information). If the subject does not tolerate ocular vasoconstrictors due to adverse reactions, continued treatment with these may be stopped; and 5) application of steroid eye drops (dexamethasone 0.1% eye drops or equivalent) during the first 3 days of each treatment cycle (i.e., first drop to be given before start of tisotumab vedotin infusion; continue treatment for 72 hours thereafter). Steroid eye drops should be administered as 1 drop in each eye, 3 times daily, for 3 days, or used in accordance with the product prescribing information. The guidelines for ocular AEs are shown in Table 6.

TABLE 6
Dose modification and toxicity management guidelines for ocular adverse events.

| | | | |
|---|---|---|---|
| Adverse Event & | | | Guidelines for Treatment |
| Toxicity Grade | Action Taken with | Action Taken with | Prescribed by the |
| (CTCAE v4.0) | Tisotumab Vedotin | pembrolizumab | Ophthalmologist |

Conjunctivitis

| | | | |
|---|---|---|---|
| Conjunctivitis gr 1 | Hold dosing until event is | Continue | Local ophthalmologist must |
| | managed effectively | | prescribe frequent dosing of |
| | Continue tisotumab vedotin at the | | preservative-free topical |
| | same dose level | | steroid drops. |
| Conjunctivitis gr 2 | Hold dosing until event has | Continue | Local ophthalmologist must |
| 1^{st} occurrence | improved to ≤ gr 1 | | prescribe frequent dosing |
| | Continue tisotumab vedotin at the | | (every second hour) of |
| | same dose level | | preservative free topical |
| Conjunctivitis gr 2 ≥ | Hold dose of tisotumab vedotin: | Continue | steroid drops in conjunction |
| 2^{nd} occurrence | If the event improves to baseline | | with preservative free |
| | within 6 weeks (calculated from | | antibiotic prophylaxis such as |
| | the onset date of the 2^{nd} grade 2 | | chloramphenicol until the |
| | event), reduce next dose of | | local ophthalmologist deems |
| | tisotumab vedotin according to | | necessary. |
| | Table 4. | | |
| | If the event does not improve to | | |
| | baseline within 6 weeks, | | |
| | permanently discontinue | | |
| | tisotumab vedotin. | | |
| Conjunctivitis ≥ gr 2 | Permanently discontinue | Continue | |
| 3^{rd} occurrence | tisotumab vedotin. | | |
| Conjunctivitis ≥ gr 3 | Permanently discontinue | Hold dosing until | |
| | tisotumab vedotin | event has | |
| | | improved to ≤ gr | |
| | | 1. Contact | |
| | | sponsor to | |
| | | determine if | |
| | | pembrolizumab | |
| | | may be | |
| | | continued. | |

Keratitis

| | | | |
|---|---|---|---|
| Keratitis ≤ gr 2 | Hold tisotumab vedotin until | Continue | The local ophthalmologist |
| 1^{st} occurrence | event has improved to ≤ gr 1 | | must prescribe frequent dosing |
| | Reduce tisotumab vedotin | | (every second hour) of |
| | according to Table 4. | | preservative free topical |
| Keratitis ≤ gr 2 | Hold tisotumab vedotin until | Continue | steroid drops in conjunction |
| 2^{nd} occurrence | event has improved to ≤ gr 1 | | with preservative free |
| | Reduce tisotumab vedotin again | | antibiotic prophylaxis such as |
| | according to Table 4. | | chloramphenicol until the |
| | | | local ophthalmologist deems |
| Keratitis ≤ gr 2 | Permanently discontinue | Continue | necessary. |
| 3^{rd} occurrence | tisotumab vedotin | | |
| Keratitis ≥ gr 3 | Permanently discontinue | Hold dosing until | |
| | tisotumab vedotin | event has improved | |
| | | to ≤ gr 1 | |

Conjunctival ulceration and ophthalmological findings of fluorescent patches must be handled as below

| | | | |
|---|---|---|---|
| Any grade | Hold tisotumab vedotin until | Continue | The local ophthalmologist |
| 1^{st} occurrence | event is managed effectively | | must prescribe frequent dosing |
| | Reduce tisotumab vedotin | | (every second hour) of |
| | according to Table 4. | | preservative free topical |
| Any grade ≥ 2^{nd} | If symptoms do not | Hold dosing until | steroid drops in conjunction |
| occurrence | stabilize/improve after dose | event has | with preservative free |
| | reduction, the subject must | improved to ≤ gr | antibiotic prophylaxis such as |
| | permanently discontinue | 1. Contact | chloramphenicol until the |
| | tisotumab vedotin. | sponsor to | local ophthalmologist deems |
| | | determine if | necessary. |
| | | pembrolizumab | |
| | | may be | |
| | | continued. | |

Symblepharon must be handled as below

| | | | |
|---|---|---|---|
| Any grade | Permanently discontinue | Hold dosing until | Consult local ophthalmologist |
| | tisotumab vedotin | event has | immediately. |
| | | improved to grade | |
| | | 0 or 1. Contact | |
| | | sponsor to | |
| | | determine if | |
| | | pembrolizumab | |
| | | may be | |
| | | continued. | |

All other ocular toxicities

| | | | |
|---|---|---|---|
| All other ocular | Hold dosing until event is | Continue. | Local ophthalmologist must |
| toxicities grade 1 | managed effectively. | | prescribe frequent dosing |
| | Continue tisotumab vedotin at the | | (every second hour) of |
| | same dose level. | | preservative free topical |
| | | | steroid drops in conjunction |
| | | | with preservative free |
| | | | antibiotic prophylaxis such as |
| | | | chloramphenicol until the |
| | | | local ophthalmologist deems |
| | | | necessary. |
| All other ocular | Hold tisotumab vedotin until | Continue. | |
| toxicities grade 2 | event is managed effectively. | | |
| 1^{st} occurrence | Reduce tisotumab vedotin | | |
| | according to Table 4. | | |
| All other ocular | Hold dose of tisotumab vedotin: | Continue. | |
| toxicities grade 2 | If the event has improved to | | |
| 2^{nd} occurrence | baseline within 6 weeks, reduce | | |
| | next dose of tisotumab vedotin | | |
| | according to Table 4. | | |
| | If the event does not improve to | | |
| | baseline within 6 weeks, | | |
| | permanently discontinue | | |
| | tisotumab vedotin. | | |
| All other ocular | Permanently discontinue | Continue. | Consult local ophthalmologist |
| toxicities grade 2 | tisotumab vedotin. | | immediately. |
| 3^{rd} occurrence | | | |

gr = grade

For AEs of peripheral neuropathy (including neuropathy peripheral; peripheral sensory neuropathy; peripheral motor neuropathy; polyneuropathy): Peripheral neuropathy is a well-known adverse reaction to treatment with platinum and taxane based chemotherapies as well as MMAE-based ADCs and is reported in approximately 35% of subjects who received treatment with tisotumab vedotin. The majority of the reported cases are grade 1-2; however peripheral neuropathy is the leading cause of permanently discontinuation of tisotumab vedotin treatment. A mitigation plan, including dose reduction (See Table 4) and dose delays, is in place to control the rates and severity of peripheral neuropathy observed in subjects treated with tisotumab vedotin. For Grade 2 and 3, or initial or worsening of pre-existing condition, hold tisotumab vedotin until event has improved to ≤grade 1 then reduce next dose according to dose reduction shown in Table 4. No action is required to be taken with pembrolizumab. For ≥Grade 4, permanently discontinue tisotumab vedotin. Contact sponsor to discuss continuation of pembrolizumab alone.

For AEs of bleeding: Bleeding events are considered of special interest due to the mode of action of tisotumab vedotin. Epistaxis is the most common reported AE, however, nearly all of the cases are grade 1. Furthermore, clinically relevant perturbations in activated partial thromboplastin time (aPTT) or prothrombin time (PT) have not been observed. Dose modification and toxicity management guidelines are in place (Table 7).

TABLE 7
Dose modification and toxicity management guidelines for adverse events
(Bleeding, Mucositis, Neutropenia, and Neuropathy) associated with the
pembrolizumab in combination with tisotumab vedotin treatment group.

| | | |
|---|---|---|
| Adverse Event | | |
| (CTCAE v4.0) | Action Taken with Tisotumab Vedotin | Action Taken with Pembrolizumab |

Bleeding Events

Control vital signs and ensure stabilization of the subject according to local standards.
Prompt evaluation to identify the underlying etiology of the bleeding event. Management should
ultimately be dictated by the underlying diagnosis.
Control laboratory coagulation and hematologic parameters including PT, aPTT, fibrinogen, platelets,
INR and hemoglobin as soon as possible.
AU Subjects

| | | |
|---|---|---|
| Any grade | Permanently discontinue tisotumab | Withhold until event resolves to |
| pulmonary or | vedotin treatment. | grade 0 or 1 |
| CNS hemorrhage ≥ | | |
| grade 2 | | |

Subjects not on anti-coagulation therapy

| 1^{st} occurrence | Hold dosing until: | | Withhold until event resolves to |
|---|---|---|---|
| Hemorrhage | a) | Bleeding has resolved. | grade 0 or 1 |
| (other)¹ ≥ grade 3 | b) | Blood hemoglobin level is stable. | |
| | c) | There is no bleeding diathesis that | |
| | | could increase the risk of continuing | |
| | | therapy. | |
| | d) | There is no anatomical or pathologic | |
| | | condition that can increase the risk of | |
| | | hemorrhage recurrence. | |

| | | |
|---|---|---|
| | When the above criteria are fulfilled the | |
| | subject can resume treatment with | |
| | tisotumab vedotin at the same dose as | |
| | prior to the event. | |
| ≥2^{nd} occurrence | Contact sponsor in order to discuss | Withhold until event resolves to |
| Hemorrhage | whether the subject may continue or must | grade 0 or 1 |
| (other)¹ ≥ grade 3 | permanently discontinue tisotumab | |
| | vedotin treatment. | |

Subjects on anti-coagulation therapy

| | | |
|---|---|---|
| INR > 3.0 | Subjects on therapeutic anticoagulation | None |
| | whose INR is >3.0 prior to infusion of | |
| | tisotumab vedotin must hold tisotumab | |
| | vedotin until INR is ≤3.0. Subjects may | |
| | resume tisotumab vedotin administration | |
| | immediately after the INR is ≤3.0. | |
| | Strongly consider holding anticoagulation | |
| | until the above parameters are met. | |
| Hemorrhage | Hold anti-coagulation therapy. | Withhold until event resolves to |
| (other)¹ ≥ grade 3 | Contact sponsor in order to discuss | grade 0 or 1 |
| | whether the subject may continue or must | |
| | permanently discontinue tisotumab | |
| | vedotin treatment. | |

¹Any other hemorrhage with the exception of pulmonary or CNS hemorrhage.

### Example 6: Cells from Multiple Tissues Exposed to Tisotumab Vedotin ADC and MMAE Undergo Cell Death and Release ATP and HMGB1

Immunogenic cell death (ICD) is a mode of apoptosis that generates immune responses against the apoptotic cancerous cells. Proteins normally found within the endoplasmic reticulum (ER) become exposed on the cell surface, leading to increased phagocytic uptake and presentation of tumor antigens to T cells in order to prime the adaptive immune system. As such, ICD induction enables the immune system to recognize and mount cytotoxic activity against tumors.

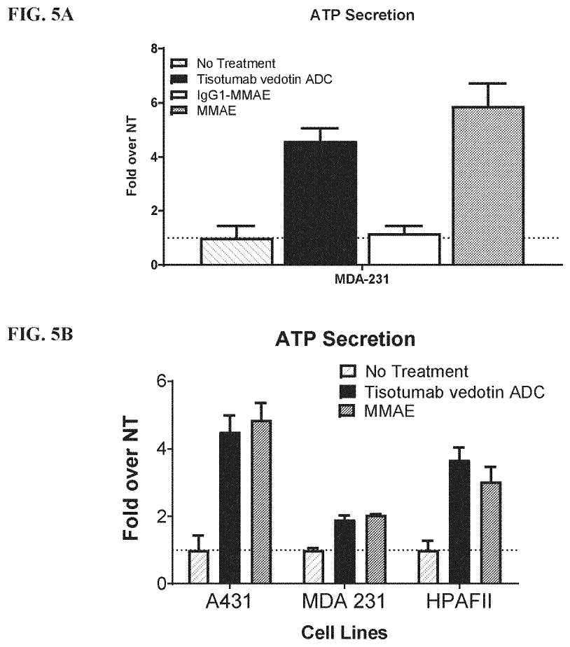

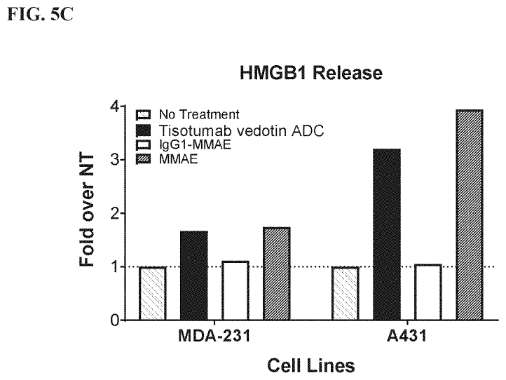

Auristatin ADC payloads disrupt the microtubule networks resulting in altered ER localization and function, which ultimately results in ER stress. Cells exposed to Tissue Factor directed antibody linked to the monomethyl auristatin E payload (MMAE), i.e., tisotumab vedotin (an antibody drug conjugate or ADC), undergo cell death and as they do release the ICD related molecules ATP (FIG. 5A) and HMGβ1 (FIG. 5C). The release of these molecules is specific to tisotumab vedotin ADC and MMAE treatment and occurs across multiple Tissue Factor positive cell lines (FIG. 5B).

### Example 7: Auristatins, Both Free and ADC-Loaded, are Able to Induce ER Stress Pathways that are Critical for Immunogenic Cell Death

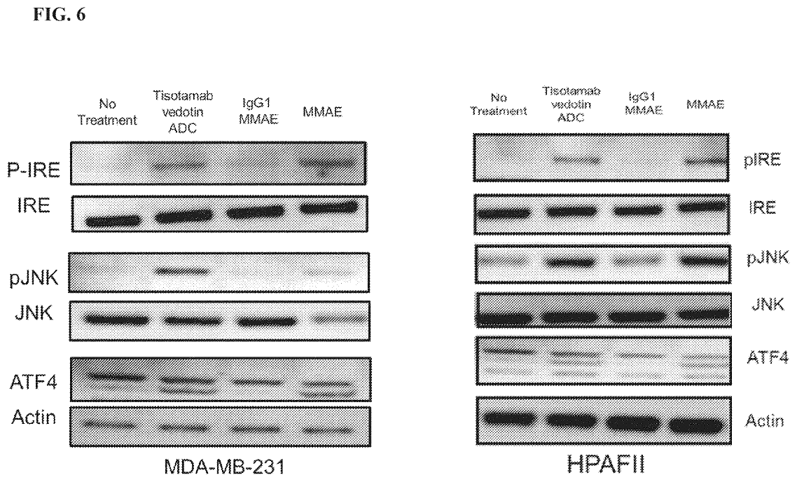

Induction of cell death and release of ICD danger signals occurs concomitant with initiation of an ER stress response. Two Tissue Factor positive cell lines, HPAFII (pancreatic carcinoma) and MDA-MB-231 (breast cell carcinoma) were exposed to tisotumab vedotin ADC, an Isotype-MMAE ADC (H00-MMAE, IgG1 MMAE), or free MMAE for 18 hours and induction of ER stress monitored by western blot analysis. Phosphorylation of inositol-requiring transmembrane kinase/endonuclease 1 (IRE1) was detected after treatment with tisotumab vedotin ADC or MMAE free drug (FIG. 6). Activation of the IRE1 downstream effector Jun N-terminal kinase (JNK) also occurred as monitored by increased phosphorylation. Furthermore, activation of the PKR-like ER kinase (PERK) secondary ER stress pathway was detected via upregulation of ATF4 cleavage. These data indicate that auristatins, both free and ADC-loaded, are able to induce ER stress pathways that are critical for ICD and the expression of tumor antigens on apoptotic cell surfaces. The ability for auristatins to prime the immune system to recognize tumor antigens opens the door for a myriad of combinatorial therapeutic options.

### Example 8: Tisotumab Vedotin ADC and MMAE Killed Tissue Factor Positive Cells Elicit Strong Chemotactic and Inflammatory Mediators from Monocyte/Macrophages after Uptake of Dead Cells

Investigations into the mechanisms of action of therapeutics for oncology extend long past cytolysis of tumor cells. The growing focus on immunotherapy highlights the processes involved in clearing dying tumor cells, as well as engaging the patient's immune system to provoke antitumor responses. The method of cell death and subsequent clearance of cell debris speaks volumes to the level of engagement and stimulation of the immune system to generate targeted responses against the tumor cells

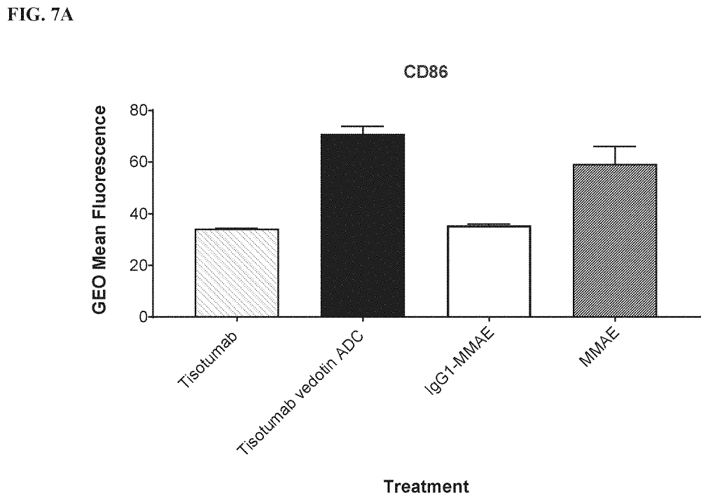

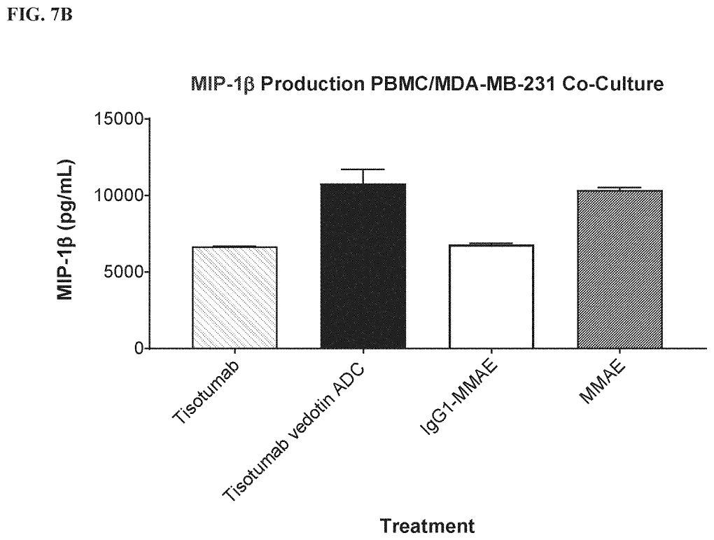

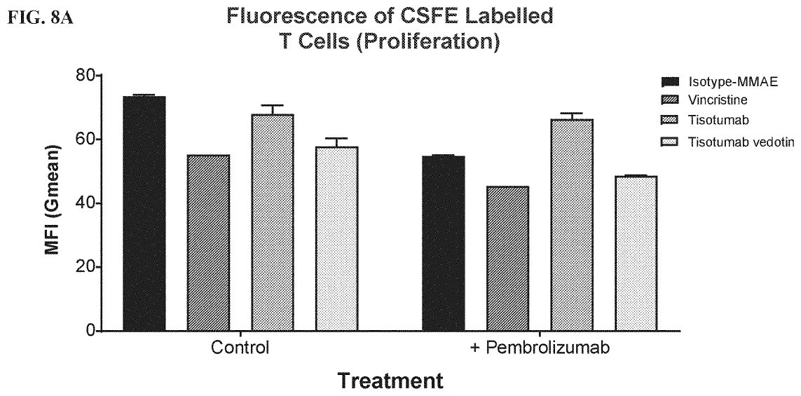

Immunogenic cell death, as mediated by MMAE, is regulated cell death that activates adaptive immune responses against antigens from dead and dying tumor cells, and allows for the generation of robust innate immune cell activation and subsequent cytotoxic T cell responses targeted towards specific tumor cell antigens. Here, we demonstrated that tisotumab vedotin ADC and MMAE killed Tissue Factor positive cells elicit strong chemotactic and inflammatory mediators from monocyte/macrophages after uptake of dead cells (FIGS. 7A and 7B). Furthermore, these monocyte/macrophages conditioned by ICD-killed cells promote activation of T cells, as evidenced by production of signature inflammatory cytokines associated with cytotoxic T cell responses.

### Example 9: Tisotumab Vedotin Induces ICD which Results in Innate Immune Cells Activation and Secondary T Cell Responses that can be Amplified with the PD1 Targeted Agent Pembrolizumab

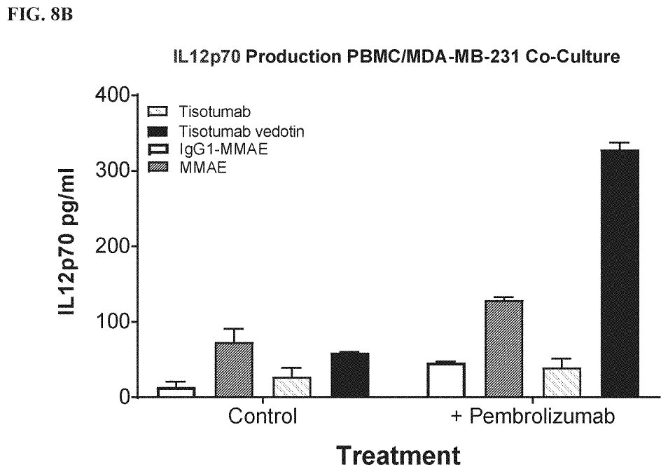

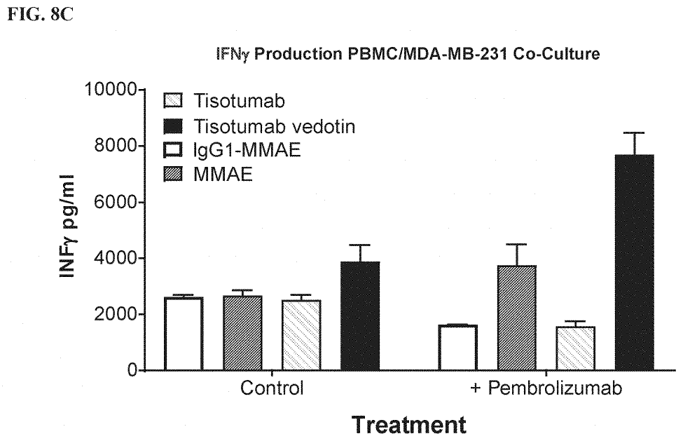

Induction of the innate immune response following exposure to cancer cells undergoing ICD sets up secondary T cell activation, which can be enhanced by concomitant pembrolizumab treatment. Tissue Factor positive MDA-MB-231 cells exposed to tisotumab vedotin or MMAE when fed to CSFE labeled human PBMCs for 48 hours drove T cell proliferation as monitored by CSFE dilution (FIG. 8A) and production of T cell specific cytokines such as IL12_{P}70 and IFNγ (FIGS. 8B and 8C). Tissue Factor targeting antibody alone or an isotype-MMAE ADC (Isotype-MMAE, IgG1-MMAE) did not elicit these responses. These data support that tisotumab vedotin induces ICD which results in innate immune cells activation and secondary T cell responses that can be amplified with pembrolizumab.

What is claimed is:

1. A method of treating cancer in a subject, the method comprising administering to the subject an antibody or an antigen-binding fragment thereof, wherein the antibody binds to Programmed Death-1 (PD-1) and inhibits PD-1 activity, and an antibody-drug conjugate that binds to tissue factor (TF), wherein the antibody-drug conjugate comprises an anti-TF antibody or an antigen-binding fragment thereof conjugated to monomethyl auristatin E, wherein the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO: 17;
(ii) a CDR-H2 comprising the amino acid sequence of SEQ ID NO: 18; and
(iii) a CDR-H3 comprising the amino acid sequence of SEQ ID NO: 19; and
wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22,
and wherein the anti-TF antibody or antigen-binding fragment thereof comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6, wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period.

2. An antibody-drug conjugate that binds to TF for use in the treatment of cancer in a subject, wherein the antibody-drug conjugate is for administration, or to be administered in combination with an anti-PD-1 antibody or an antigen-binding fragment thereof, or
an anti-PD-1 antibody or an antigen-binding fragment thereof for use in the treatment of cancer in a subject, wherein the anti-PD-1 antibody is for administration, or to be administered in combination with an antibody-drug conjugate that binds to TF;
wherein the antibody-drug conjugate comprises an anti-TF antibody or an antigen-binding fragment thereof conjugated to monomethyl auristatin E,
wherein the anti-PD-1 antibody or the antigen-binding fragment thereof inhibits PD-1 activity,
wherein the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO: 17;
(ii) a CDR-H2 comprising the amino acid sequence of SEQ ID NO: 18; and
(iii) a CDR-H3 comprising the amino acid sequence of SEQ ID NO: 19; and
wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22; and
wherein the anti-TF antibody or antigen-binding fragment thereof comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO:2; and
(iii) a CDR-H3 comprising the amino acid sequence of SEQ ID NO: 3; and
wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6,
wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period.

3. Use of an antibody-drug conjugate that binds to TF for the manufacture of a medicament for treating cancer in a subject, wherein the medicament is for use in combination with an anti-PD-1 antibody or an antigen-binding fragment thereof,
or
use of an anti-PD-1 antibody or an antigen-binding fragment thereof for the manufacture of a medicament for treating cancer in a subject, wherein the medicament is for use in combination with an antibody-drug conjugate that binds to TF,
wherein the antibody-drug conjugate comprises an anti-TF antibody or an antigen-binding fragment thereof conjugated to monomethyl auristatin E,
wherein the anti-PD-1 antibody or the antigen-binding fragment thereof inhibits PD-1 activity,
wherein the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO: 17;
(ii) a CDR-H2 comprising the amino acid sequence of SEQ ID NO: 18; and
(iii) a CDR-H3 comprising the amino acid sequence of SEQ ID NO: 19; and
wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22; and
wherein the anti-TF antibody or antigen-binding fragment thereof comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO:2; and
(iii) a CDR-H3 comprising the amino acid sequence of SEQ ID NO: 3; and
wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6,
wherein the antibody-drug conjugate is administered at a dose ranging from about 0.5 mg/kg to about 2.1 mg/kg, wherein the antibody-drug conjugate is administered once about every 1 week for 3 consecutive weeks followed by about a 1 week rest period without any administration of the antibody-drug conjugate so that each cycle time is about 28 days including the resting period.

4. The method or use of any one of claims 1-3, wherein the antibody-drug conjugate is administered at a dose of about 0.65 mg/kg.

5. The method or use of any one of claims 1-3, wherein the antibody-drug conjugate is administered at a dose of 0.65 mg/kg.

6. The method or use of any one of claims 1-3, wherein the antibody-drug conjugate is administered at a dose of about 0.7 mg/kg.

7. The method or use of any one of claims 1-3, wherein the antibody-drug conjugate is administered at a dose of 0.7 mg/kg.

8. The method or use of any one of claims 1-3, wherein the antibody-drug conjugate is administered at a dose of about 0.8 mg/kg.

9. The method or use of any one of claims 1-3, wherein the antibody-drug conjugate is administered at a dose of 0.8 mg/kg.

10. The method or use of any one of claims 1-3, wherein the antibody-drug conjugate is administered at a dose of about 0.9 mg/kg.

11. The method or use of any one of claims 1-3, wherein the antibody-drug conjugate is administered at a dose of 0.9 mg/kg.

12. The method or use of any one of claims 1-3, wherein the antibody-drug conjugate is administered at a dose of about 1.0 mg/kg.

13. The method or use of any one of claims 1-3, wherein the antibody-drug conjugate is administered at a dose of 1.0 mg/kg.

14. The method or use of any one of claims 1-3, wherein the antibody-drug conjugate is administered at a dose of about 1.1 mg/kg.

15. The method or use of any one of claims 1-3, wherein the antibody-drug conjugate is administered at a dose of 1.1 mg/kg.

16. The method or use of any one of claims 1-3, wherein the antibody-drug conjugate is administered at a dose of about 1.2 mg/kg.

17. The method or use of any one of claims 1-3, wherein the antibody-drug conjugate is administered at a dose of 1.2 mg/kg.

18. The method or use of any one of claims 1-3, wherein the antibody-drug conjugate is administered at a dose of about 1.3 mg/kg.

19. The method or use of any one of claims 1-3, wherein the antibody-drug conjugate is administered at a dose of 1.3 mg/kg.

20. The method or use of any one of claims 1-3, wherein the antibody-drug conjugate is administered at a dose of about 1.4 mg/kg.

21. The method or use of any one of claims 1-3, wherein the antibody-drug conjugate is administered at a dose of 1.4 mg/kg.

22. The method or use of any one of claims 1-3, wherein the antibody-drug conjugate is administered at a dose of about 1.5 mg/kg.

23. The method or use of any one of claims 1-3, wherein the antibody-drug conjugate is administered at a dose of 1.5 mg/kg.

24. The method or use of any one of claims 1-23, wherein the antibody-drug conjugate is administered once every 1 week for 3 consecutive weeks followed by a 1 week rest period without any administration of the antibody-drug conjugate so that each cycle time is 28 days including the resting period.

25. The method or use of any one of claims 1-23, wherein the antibody-drug conjugate is administered on about days 1, 8, and 15 of about a 4-week cycle.

26. The method or use of any one of claims 1-23, wherein the antibody-drug conjugate is administered on days 1, 8, and 15 of a 4-week cycle.

27. The method or use of any one of claims 1-26, wherein the anti-PD-1 antibody or antigen-binding fragment thereof is administered at a flat dose ranging from about 50 mg to about 500 mg.

28. The method or use of any one of claims 1-27, wherein the anti-PD-1 antibody or antigen-binding fragment thereof is administered at a flat dose of about 200 mg.

29. The method or use of any one of claims 1-27, wherein the anti-PD-1 antibody or antigen-binding fragment thereof is administered at a flat dose of 200 mg.

30. The method or use of any one of claims 1-27, wherein the anti-PD-1 antibody or antigen-binding fragment thereof is administered at a flat dose of about 400 mg.

31. The method or use of any one of claims 1-27, wherein the anti-PD-1 antibody or antigen-binding fragment thereof is administered at a flat dose of 400 mg.

32. The method or use of any one of claims 1-31, wherein the anti-PD-1 antibody or antigen-binding fragment thereof is administered once about every 1 week, once about every 2 weeks, once about every 3 weeks, once about every 4 weeks, once about every 5 weeks, or once about every 6 weeks.

33. The method or use of claim 32, wherein the anti-PD-1 antibody or antigen-binding fragment thereof is administered once about every 3 weeks.

34. The method or use of claim 32, wherein the anti-PD-1 antibody or antigen-binding fragment thereof is administered once every 3 weeks.

35. The method or use of claim 32, wherein the anti-PD-1 antibody or antigen-binding fragment thereof is administered once about every 6 weeks.

36. The method or use of claim 30, wherein the anti-PD-1 antibody or antigen-binding fragment thereof is administered once every 6 weeks.

37. The method or use of any one of claims 1-32, wherein the anti-PD-1 antibody or antigen-binding fragment thereof is administered on about day 1 of about a 21-day cycle.

38. The method or use of any one of claims 1-32, wherein the anti-PD-1 antibody or antigen-binding fragment thereof is administered on day 1 of a 21-day cycle.

39. The method or use of any one of claims 1-32, wherein the anti-PD-1 antibody or antigen-binding fragment thereof is administered on about day 1 of about a 6-week cycle.

40. The method or use of any one of claims 1-32, wherein the anti-PD-1 antibody or antigen-binding fragment thereof is administered on day 1 of a 6-week cycle.

41. The method or use of any one of claims 1-40, wherein the cancer is breast cancer.

42. The method or use of claim 41, wherein the breast cancer is ER+/HER2− breast cancer or triple negative breast cancer.

43. The method or use of any one of claims 1-40, wherein the cancer is cervical cancer.

44. The method or use of claim 43, wherein the subject is not a candidate for curative therapy.

45. The method or use of claim 44, wherein curative therapy comprises radiotherapy and/or exenterative surgery.

46. The method or use of claim 45, wherein the subject has not received prior systemic therapy for the cervical cancer.

47. The method or use of any one of claims 43-46, wherein the cervical cancer is a non-squamous cell carcinoma, an adenocarcinoma, an adenosquamous carcinoma or a squamous cell carcinoma.

48. The method or use of claim 47, wherein the cervical cancer is an adenocarcinoma.

49. The method or use of claim 47, wherein the cervical cancer is an adenosquamous carcinoma.

50. The method or use of claim 47, wherein the cervical cancer is a squamous cell carcinoma.

51. The method or use of claim 47, wherein the cervical cancer is a non-squamous cell carcinoma.

52. The method or use of any one of claims 43-51, wherein the cervical cancer is an advanced stage cervical cancer.

53. The method or use of claim 52, wherein the advanced stage cervical cancer is a stage 3 or stage 4 cervical cancer.

54. The method or use of claim 52 or 53, wherein the advanced stage cervical cancer is metastatic cervical cancer.

55. The method or use of any one of claims 41-54, wherein the cervical cancer is recurrent cervical cancer.

56. The method or use of any one of claims 1-55, wherein the anti-TF antibody or antigen-binding fragment thereof of the antibody-drug conjugate is a monoclonal antibody or a monoclonal antigen-binding fragment thereof.

57. The method or use of any one of claims 1-56, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.

58. The method or use of any one of claims 1-57, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO: 8.

59. The method or use of any one of claims 1-58, wherein the anti-TF antibody of the antibody-drug conjugate is tisotumab.

60. The method or use of any one of claims 1-59, wherein the antibody-drug conjugate further comprises a linker between the anti-TF antibody or antigen-binding fragment thereof and the monomethyl auristatin E.

61. The method or use of claim 59, wherein the linker is a cleavable peptide linker.

62. The method or use of claim 61, wherein the cleavable peptide linker has a formula: -MC-vc-PAB-, wherein:
a) MC is: 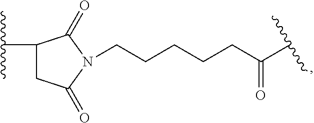 [SMILES: *C(=O)CCCCCN1C(=O)CC(*)C1=O]
b) vc is the dipeptide valine-citrulline, and
c) PAB is:

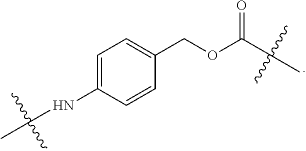 [SMILES: *Nc1ccc(COC(*)=O)cc1]

63. The method or use of any one of claims 60-62, wherein the linker is attached to sulphydryl residues of the anti-TF antibody or antigen-binding fragment thereof obtained by partial reduction or full reduction of the anti-TF antibody or antigen-binding fragment thereof.

64. The method or use of claim 63, wherein the linker is attached to MMAE, wherein the antibody-drug conjugate has the following structure: 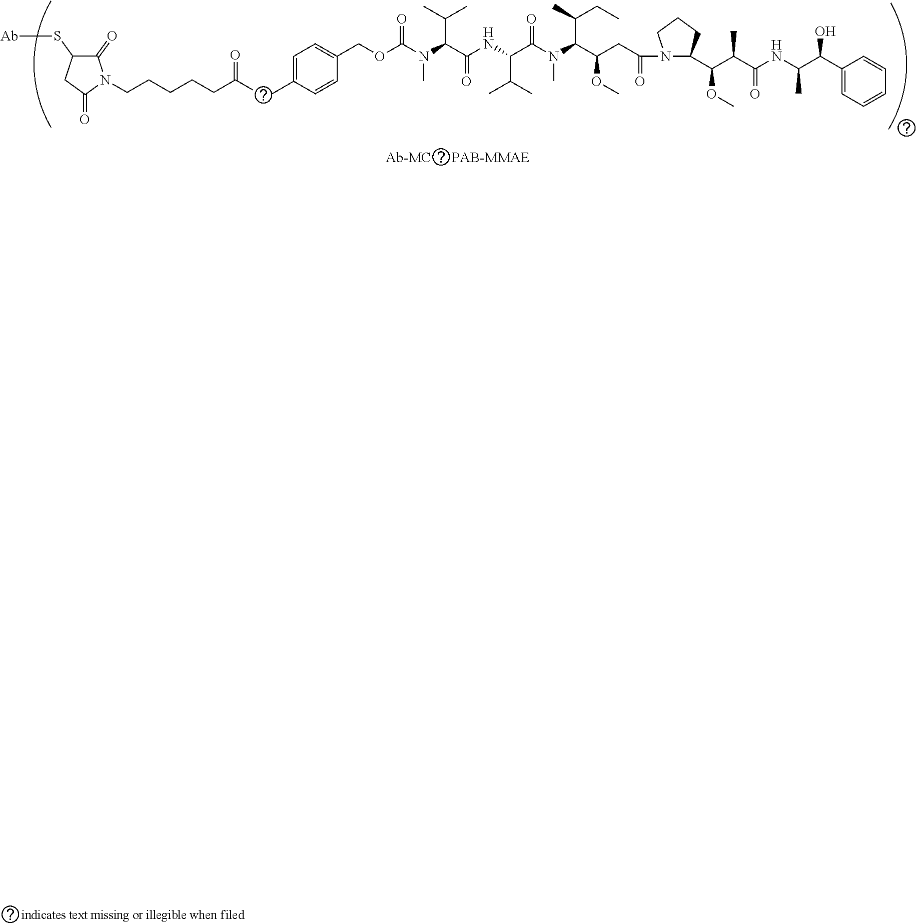
wherein p denotes a number from 1 to 8, S represents a sulphydryl residue of the anti-TF antibody, and Ab designates the anti-TF antibody or antigen-binding fragment thereof.

65. The method or use of claim 64, wherein the average value of p in a population of the antibody-drug conjugates is about 4.

66. The method or use of any one of claims 1-65, wherein the antibody-drug conjugate is tisotumab vedotin.

67. The method or use of any one of claims 1-66, wherein the route of administration for the antibody-drug conjugate is intravenous.

68. The method or use of any one of claims 1-67, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.

69. The method or use of any one of claims 1-68, wherein the anti-PD-1 antibody comprises a heavy chain variable region comprising the amino acid sequence of SEQ ID NO:31 and a light chain variable region comprising the amino acid sequence of SEQ ID NO:32.

70. The method or use of any one of claims 1-69, wherein the anti-PD-1 antibody comprises a heavy chain comprising the amino acid sequence of SEQ ID NO:33 and a light chain comprising the amino acid sequence of SEQ ID NO:34.

71. The method or use of any one of claims 1-70, wherein the anti-PD-1 antibody is pembrolizumab.

72. The method or use of any one of claims 1-71, wherein the route of administration for the anti-PD-1 antibody or antigen-binding fragment thereof is intravenous or subcutaneous.

73. The method or use any one of claims 1-71, wherein the route of administration for the anti-PD-1 antibody or antigen-binding fragment thereof is intravenous.

74. The method or use of any one of claims 1-71, wherein the route of administration for the anti-PD-1 antibody or antigen-binding fragment thereof is subcutaneous.

75. The method or use of any one of claims 1-74, wherein the anti-PD-1 antibody or antigen-binding fragment thereof and the antibody-drug conjugate are administered sequentially.

76. The method or use of any one of claims 1-74, wherein the anti-PD-1 antibody or antigen-binding fragment thereof and the antibody-drug conjugate are administered simultaneously.

77. The method or use of any one of claims 1-76, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.

78. The method or use of any one of claims 1-77, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.

79. The method or use of any one of claims 1-78, wherein the subject has a tumor that expresses PD-L1 (TPS≥1).

80. The method or use of any one of claims 1-79, wherein the subject has a tumor that has high PD-L1 expression (TPS≥50).

81. The method or use of any one of claims 1-80, wherein the subject has a tumor that expresses PD-L1 (CPS≥1).

82. The method or use of any one of claims 1-77, wherein a tumor derived from the cancer comprises one or more cells that express PD-L1, PD-L2, or both PD-L1 and PD-L2.

83. The method or use of any one of claims 1-82, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.

84. The method or use of any one of claims 1-83, wherein one or more therapeutic effects in the subject is improved after administration of the antibody-drug conjugate and the anti-PD-1 antibody or antigen-binding fragment thereof relative to a baseline.

85. The method or use of claim 84, wherein the one or more therapeutic effects is selected from the group consisting of: size of a tumor derived from the cancer, objective response rate, duration of response, time to response, progression free survival, and overall survival.

86. The method or use of any one of claims 1-85, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.

87. The method or use of any one of claims 1-86, wherein the objective response rate is at least about 20%, at least about 25%, at least about 30%, at least about 35%, at least about 40%, at least about 45%, at least about 50%, at least about 60%, at least about 70%, or at least about 80%.

88. The method or use of any one of claims 1-87, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.

89. The method or use of any one of claims 1-88, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.

90. The method or use of any one of claims 1-89, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.

91. The method or use of any one of claims 1-90, wherein the subject has one or more adverse events and is further administered an additional therapeutic agent to eliminate or reduce the severity of the one or more adverse events.

92. The method or use of any one of claims 1-91, wherein the subject is at risk of developing one or more adverse events and is further administered an additional therapeutic agent to prevent or reduce the severity of the one or more adverse events.

93. The method or use of any one of claims 91-92, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.

94. The method or use of any one of claims 91-93, wherein the one or more adverse events is a grade 3 or greater adverse event.

95. The method or use of any one of claims 91-93, wherein the one or more adverse events is a serious adverse event.

96. The method or use of any one of claims 92-93, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.

97. The method or use of any one of claims 1-96, wherein the subject is a human.

98. The method or use of any one of claims 1-97, wherein the antibody-drug conjugate is in a pharmaceutical composition comprising the antibody-drug conjugate and a pharmaceutical acceptable carrier.

99. The method or use of any one of claims 1-98, wherein the anti-PD-1 antibody or antigen-binding fragment thereof is in a pharmaceutical composition comprising the anti-PD-1 antibody or antigen-binding fragment thereof and a pharmaceutical acceptable carrier.

100. A kit comprising:
(a) a dosage ranging from about 50 mg to about 500 mg of an antibody or an antigen-binding fragment thereof, wherein the antibody binds to Programmed Death-1 (PD-1) and inhibits PD-1 activity, wherein the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO: 17;
(ii) a CDR-H2 comprising the amino acid sequence of SEQ ID NO: 18; and
(iii) a CDR-H3 comprising the amino acid sequence of SEQ ID NO: 19; and
wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22;
(b) a dosage ranging from about 5 mg to about 200 mg of an antibody-drug conjugate that binds to tissue factor (TF), wherein the antibody-drug conjugate comprises an anti-TF antibody or an antigen-binding fragment thereof conjugated to monomethyl auristatin E, wherein the anti-TF antibody or antigen-binding fragment thereof comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO:2; and
(iii) a CDR-H3 comprising the amino acid sequence of SEQ ID NO: 3; and
wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6; and
(c) instructions for use of the anti-PD-1 antibody or antigen-binding fragment thereof and the antibody drug conjugate according to the method of any one of claims 1-99.

101. The kit of claim 100, wherein the anti-PD-1 antibody or antigen-binding fragment thereof is pembrolizumab.

102. The kit of claim 101, wherein the dose of the pembrolizumab is 200 mg.

103. The kit of claim 102, wherein the dose of the pembrolizumab is 400 mg.

104. The kit of any one of claims 100-103, wherein the antibody-drug conjugate is tisotumab vedotin.


SEQUENCE LISTING (34 sequences)

SEQ ID NO: 1 - protein, 8 aa, Artificial Sequence
  Synthetic Construct
GFTFSNYA

SEQ ID NO: 2 - protein, 8 aa, Artificial Sequence
  Synthetic Construct
ISGSGDYT

SEQ ID NO: 3 - protein, 11 aa, Artificial Sequence
  Synthetic Construct
ARSPWGYYLDS

SEQ ID NO: 4 - protein, 6 aa, Artificial Sequence
  Synthetic Construct
QGISSR

SEQ ID NO: 5 - protein, 3 aa, Artificial Sequence
  Synthetic Construct
AAS

SEQ ID NO: 6 - protein, 9 aa, Artificial Sequence
  Synthetic Construct
QQYNSYPYT

SEQ ID NO: 7 - protein, 118 aa, Artificial Sequence
  Synthetic Construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 8 - protein, 107 aa, Artificial Sequence
  Synthetic Construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 9 - protein, 25 aa, Artificial Sequence
  Synthetic Construct
EVQLLESGGGLVQPGGSLRLSCAAS

SEQ ID NO: 10 - protein, 17 aa, Artificial Sequence
  Synthetic Construct
MSWVRQAPGKGLEWVSS

SEQ ID NO: 11 - protein, 38 aa, Artificial Sequence
  Synthetic Construct
YYTDSVKGRFTISRDNSKNTLYLQMNSLRAEDTAVYYC

SEQ ID NO: 12 - protein, 11 aa, Artificial Sequence
  Synthetic Construct
WGQGTLVTVSS

SEQ ID NO: 13 - protein, 26 aa, Artificial Sequence
  Synthetic Construct
DIQMTQSPPSLSASAGDRVTITCRAS

SEQ ID NO: 14 - protein, 17 aa, Artificial Sequence
  Synthetic Construct
LAWYQQKPEKAPKSLIY

SEQ ID NO: 15 - protein, 36 aa, Artificial Sequence
  Synthetic Construct
SLQSGVPSRFSGSGSGTDFTLTISSLQPEDFATYYC

SEQ ID NO: 16 - protein, 10 aa, Artificial Sequence
  Synthetic Construct
FGQGTKLEIK

SEQ ID NO: 17 - protein, 5 aa, Artificial Sequence
  Synthetic Construct
NYYMY

SEQ ID NO: 18 - protein, 17 aa, Artificial Sequence
  Synthetic Construct
GINPSNGGTNFNEKFKN

SEQ ID NO: 19 - protein, 11 aa, Artificial Sequence
  Synthetic Construct
RDYRFDMGFDY

SEQ ID NO: 20 - protein, 15 aa, Artificial Sequence
  Synthetic Construct
RASKGVSTSGYSYLH

SEQ ID NO: 21 - protein, 7 aa, Artificial Sequence
  Synthetic Construct
LASYLES

SEQ ID NO: 22 - protein, 9 aa, Artificial Sequence
  Synthetic Construct
QHSRDLPLT

SEQ ID NO: 23 - protein, 30 aa, Artificial Sequence
  Synthetic Construct
QVQLVQSGVEVKKPGASVKVSCKASGYTFT

SEQ ID NO: 24 - protein, 14 aa, Artificial Sequence
  Synthetic Construct
WVRQAPGQGLEWMG

SEQ ID NO: 25 - protein, 32 aa, Artificial Sequence
  Synthetic Construct
RVTLTTDSSTTTAYMELKSLQFDDTAVYYCAR

SEQ ID NO: 26 - protein, 11 aa, Artificial Sequence
  Synthetic Construct
WGQGTTVTVSS

SEQ ID NO: 27 - protein, 23 aa, Artificial Sequence
  Synthetic Construct
EIVLTQSPATLSLSPGERATLSC

SEQ ID NO: 28 - protein, 15 aa, Artificial Sequence
  Synthetic Construct
WYQQKPGQAPRLLIY

SEQ ID NO: 29 - protein, 32 aa, Artificial Sequence
  Synthetic Construct
GVPARFSGSGSGTDFTLTISSLEPEDFAVYYC

SEQ ID NO: 30 - protein, 10 aa, Artificial Sequence
  Synthetic Construct
FGGGTKVEIK

SEQ ID NO: 31 - protein, 120 aa, Artificial Sequence [sequence omitted]

SEQ ID NO: 32 - protein, 111 aa, Artificial Sequence [sequence omitted]

SEQ ID NO: 33 - protein, 446 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC